(19) United States
(12) Patent Application Publication
(10) Pub. No.: US 2026/0028407 A1
(43) Pub. Date: Jan. 29, 2026

(54) MODIFIED ANTI-GALECTIN-9 ANTIBODY AND USES THEREOF

(71) Applicants: FibroGen, Inc., San Francisco, CA (US); HiFiBiO, Inc., Cambridge, MA (US)

(72) Inventors: David Olsen, Palo Alto, CA (US); Qian Zhang, Hong Kong (CN); Ling Dong, Hong Kong (CN); Tom Pohl, Santa Fe, NM (US); Fortunato Ferrara, Santa Fe, NM (US); Andrew Bradbury, Santa Fe, NM (US); Mitchell C. Brenner, San Francisco, CA (US); Gail Walkinshaw, San Francisco, CA (US)

(21) Appl. No.: 18/995,102

(22) PCT Filed: Jul. 14, 2023
(86) PCT No.: PCT/US2023/070259

§ 371 Date: Jan. 15, 2025

Related U.S. Application Data
(60) Provisional application No. 63/389,607, filed on Jul. 15, 2022.
(60) Provisional application No. 63/389,610, filed on Jul. 15, 2022.

(51) Int. Cl. C07K 16/28; A61K 39/00; A61K 45/06; A61P 35/00; G01N 33/68

(52) U.S. Cl. CPC C07K 16/2851; A61K 45/06; A61P 35/00; G01N 33/6872; A61K 2039/505; C07K 2317/24; C07K 2317/565; C07K 2317/734; C07K 2317/76; C07K 2317/92; G01N 2333/7056; G01N 2800/52

(57) ABSTRACT

Provided herein are affinity matured anti-galectin-9 (Gal9) antibodies. The antibodies have improved binding affinity to Gal9. Also provided herein are method of use of the affinity matured anti-Gal9 antibodies, including methods of treatment of cancer, methods of rescuing or promoting effector T cell proliferation, methods of enhancing effector T cell activity, and/or of identifying and treating cancer in a subject including the administration of the anti-Gal9 antibodies described herein. Also provided are polynucleotides encoding the heavy chain or the light chain or the antigen-binding portion thereof described herein, and vectors, especially expression vectors, including the polynucleotides described herein.

### CROSS-REFERENCE TO RELATED APPLICATIONS

This application claims benefit of priority under 35 U.S.C. § 119(e) of U.S. Provisional Applications No. 63/389,607, filed Jul. 15, 2022, and No. 63/389,610, filed Jul. 15, 2022. The disclosure of the prior applications is considered part of and are herein incorporated by reference in the disclosure of this application in their entirety.

### INCORPORATION OF SEQUENCE LISTING

The material in the accompanying sequence listing is hereby incorporated by reference into this application. The accompanying sequence listing xml file, name 148640-004302_SL.xml, was created on Jul. 13, 2023, and is 80,112 bytes.

### SUMMARY OF THE INVENTION

The present invention relates generally to anti-galectin-9 antibodies, and more specifically to anti-galectin-9 antibodies for use for the treatment of cancer.

Galectin-9 (or Gal9) is a member of the galectin (or type-S lectin) family of proteins having at least 15 members in vertebrates, including 10 in humans. Gal9 is a soluble 34-39 kDa protein without a leader peptide yet is nevertheless secreted via a non-classical mechanism. It interacts preferentially with beta-galactoside residues of glycoproteins and glycolipids. In humans, Gal9 exists in three isoforms, long, medium, and short.

Gal9, a member of the lectin family of β-galactoside binding proteins, contains 2 distinct carbohydrate recognition domains (CRDs) connected by a flexible linker (tandem repeat galectin). Gal9 can simultaneous bind/ligate two ligands promoting the association or organization of two or more monomeric receptors leading to alter signaling and possibly the rate of receptor endocytosis. Several cell surface binding partners have been identified including Tim3, PD-1, VISTA, Dectin-1, CD44, CD206, 4-1BB (CD137), TNFRSF25, PDI, and others. Gal9 plays an important role regulating the activity of several immune cell types creating an immunosuppressive environment in the tumor microenvironment (TME). Notably, Gal9 (i) induces apoptosis of CD4+ and CD8+ T cells, (ii) suppresses Th17 cell development, (iii) induces suppressive Treg expansion, (iv) promotes expansion of myeloid-derived suppressor cells, (v) promotes M2-polarized tumor associated macrophage (TAM) phenotype, and (vi) impairs natural killer (NK) cell function. Several of these Gal9 activities have been confirmed with studies in Gal9 knockout mice.

Gal9 is one of the most studied ligands for T-cell immunoglobulin and mucin-domain containing-3 (TIM-3), also known as hepatitis A virus cellular receptor 2 (HAVCR2), and is expressed on various hematological malignancies, such as chronic lymphocytic leukemia (CLL), myelodysplastic syndrome (MDS), Hodgkin and Non-Hodgkin lymphomas, acute myeloid leukemia (AML), as well as solid tumors such as lung cancer, breast cancer, and hepatocellular carcinoma.

Interaction of Gal9 with TIM-3/HAVCR2 has been found to attenuate T-cell expansion and effector function in the tumor microenvironment and in chronic infections. Moreover, Gal9 contributes to tumorigenesis by tumor cell transformation, cell-cycle regulation, angiogenesis, and cell adhesion.

Gal9 is also directly expressed by regulatory T lymphocytes (or Tregs), and its expression is increased during Treg activation. Meanwhile, Gal9 is very weakly expressed by effector T lymphocytes (such as CD8⁺ cytotoxic T lymphocytes), and this expression disappears during effector T lymphocyte activation. Inhibition of Gal9 by an anti-Gal9 antibody has been found to inhibit the suppressor activity of Tregs.

Wu et al. (Immunity 41 (2): 270-282, 2014) reported that Gal9 is critical in regulating the immune response. There are two subsets of Treg cells, “natural” Treg (nTreg) cells and “induced” Treg (iTreg) cells. Gal9 is highly expressed by iTreg cells and is crucial for the generation and function of iTreg cells, but not nTreg cells. Gal9 expression in iTreg cells is driven by the transcription factor Smad3, forming a feed-forward loop, which further promotes Foxp3 expression. Gal9 increases iTreg cell stability and function by directly binding to its receptor CD44, which forms a complex with transforming growth factor-β (TGF-β) receptor I (TGF-βRI) and activated Smad3. Gal9 signaling was further found to regulate iTreg cell induction by dominantly acting through the CNS1 region of the Foxp3 locus. Exogenous Gal9, in addition to being an effector molecule for Treg cells, acts synergistically with TGF-β to enforce iTreg cell differentiation and maintenance.

Various types of T lymphocytes normally develop into, for example “effector” cells or effector T lymphocytes, which will fulfil specialized immune functions for defending the host organism. Thus, the CD4⁺ T lymphocytes or auxiliary T lymphocytes, secrete major cytokines that assist the B lymphocytes in their humoral function (the production of specific antibodies) and the CD8⁺ T lymphocytes in their cytotoxic activity.

Tregs constitutively overexpress the CD4 and CD25 molecules (hence they are also called “CD4⁺CD25⁺”) and the Foxp3 transcription factor. This small percentage of CD4⁺CD25⁺ T lymphocytes negatively regulates the actors of the immune response that would have recognized various autoantigens by their T-cell receptors (TCRs). Tregs thus fulfil a major role in the physiology of the immune system by protecting the organism against the emergence of autoimmune illnesses.

The Tregs exert immunosuppressor activity on effector T lymphocytes. Such activity, once activated, in a pathological situation such as tumor, promotes tumor growth. Thus, the suppressor activity of Tregs can be understood as an activity that reduces the anti-tumoral immune responses, by inhibiting the function of effector T lymphocytes.

Gal9 is mainly associated with tumor immunosuppression resulting from interaction with different immune receptors. For example, Gal9 inhibits Th1 responses and induces peripheral tolerance, as evidenced by decreased Th1 apoptosis upon Gal9 blockade, increased susceptibility of Gal9 knock-out mice to collagen-induced arthritis (CIA), and prolonged graft survival and autoimmune disease (AID) suppression upon Gal9 administration. Gal9 also regulates peripheral NK cell function to promote materno-fetal tolerance, promotes the expansion of myeloid-derived suppressor cells (MDSCs), and synergizes with TGF-β to promote Treg expansion.

High expression of Gal9 is evident in several solid tumors and hematological malignancies, both in patients' tumors and blood, and correlates with aggressive disease and poor survival outcomes. More recently, several published reports provided support for a combination therapy approach employing anti-Gal9 antibodies with currently used immunotherapies including immune checkpoint inhibitor therapy.

Yang et al. (Nature Commun. 12, 832, 2021) demonstrated that (i) PD-1 binds to Gal9 C-CRD, and that Gal9 and PD-L1 bind to different sites on PD-1; (ii) pembrolizumab does not block Gal9 binding to PD-1; (iii) the complex of Tim-3 and PD-1/gal9 C-CRD contributes to the persistence of exhausted T cells by limiting Tim3-Gal9 driven apoptosis; and (iv) combined treatment with anti-PD-1 and anti-Gal9 antibodies may provide a superior anti-tumor effect and general outcome (e.g. survival), as compared to each therapy used individually. Further, Yang et al., supra generated 16,291 immune cell transcriptomes from 48 tumor samples collected from 32 melanoma patients treated with anti-PD1 therapy and evaluated them for Gal9 expression and its association with treatment outcome. Tumor infiltrating lymphocytes (TILs) from anti-PD1 non-responders expressed much higher levels of gal9 as compared to patients responding to therapy.

Limagne et al., (Oncoimmunology 8: e1564505, 2019) evaluated a population of metastatic non-small cell lung cancer (mNSCLC) patients and found accumulation of Tim3+ lymphoid cells and Gal9+ monocytic-myeloid-derived suppressive cells (mMDSC) was associated with PD-1 blockade resistance.

Li et al. (Immunotherapy 15, 135, 2023) have shown that Gal9 and PD-L1 levels were higher in pancreatic ductal adenocarcinoma (PDAC) tumor compared to normal tissue and levels were significantly associated with tumor invasion of lymph nodes and tumor node metastasis staging. In a murine KPC model (an established and clinically relevant model of PDAC) significant tumor growth inhibition was observed in anti-PD-L1 and anti-Gal9 combination therapy group compared to anti-PD-L1 or anti-Gal9 alone or control groups.

Taken together, these findings suggest that in certain human cancers combined anti-Gal9 and anti-PD1 therapy could be an effective treatment regime.

Circulating levels of Gal9 are significantly higher in certain cancer patients compared to healthy controls. Therefore, there is still an unmet need for anti-Gal9 antibodies for use for the treatment of cancer.

Aspects of the invention described herein relate to antibodies directed against Gal9 and the use thereof for the treatment of diseases associated with the suppressor activity of regulatory T lymphocytes (Tregs).

In one aspect, the invention provides an isolated monoclonal antibody or an antigen-binding fragment thereof comprising (a) a heavy chain variable region (HCVR) comprising (i) a HCVR CDR1 sequence having the amino acid sequence GYX₁FX₂X₃YTIH (SEQ ID NO:1), wherein X₁ is selected from T, E, P, or A; X₂ is selected from T, G, or S; and X₃ is selected from E, S, or D; (ii) a HCVR CDR2 sequence having the amino acid sequence WFYPGSGSTX₄YAQKFQG (SEQ ID NO:8), wherein X₄ is selected from E, W, or V; and (iii) a HCVR CDR3 sequence having the amino acid sequence HGGYDGFDY (SEQ ID NO:12); and (b) a light chain variable region (LCVR) comprising (i) a LCVR CDR1 sequence having the amino acid sequence KSSX₅X₆X₇LX₈X₉X₁₀X₁₁X₁₂X₁₃NX₁₄LA (SEQ ID NO:13), wherein X₅ is selected from Q or R; X₆ is selected from S or N; X₇ is selected from L, V, or I; X₈ is selected from Y or W; X₉ is selected from S or P; X₁₀ is selected from N, S, P, or A; X₁₁ is selected from N or H; X₁₂ is selected from Q, N, or Y; X₁₃ is selected from K or R; and X₁₄ is selected from Y or H; (ii) a LCVR CDR2 sequence having the amino acid sequence WX₁₅SX₁₆RX₁₇X₁₈, wherein X₁₅ is selected from A or G; X₁₆ is selected from T, N, M, or A; X₁₇ is selected from G or E; and X₁₈ is selected from S, E, Y, P, or T; and (iii) a LCVR CDR3 sequence having the amino acid sequence QQYYX₁₉X₂₀PFT (SEQ ID NO:30), wherein X₁₉ is selected from S or F; and X₂₀ is selected from Y or F, with the proviso that the antibody does not comprise HCVR CDR1 sequence GYTFTEYTIH (SEQ ID NO:2), HCVR CDR2 sequence WFYPGSGSTEYAQKFQG (SEQ ID NO:9), HCVR CDR3 sequence HGGYDGFDY (SEQ ID NO:12), LCVR CDR1 sequence KSSQSLLYSNNQKNYLA (SEQ ID NO:14), LCVR CDR2 sequence WASTRGS (SEQ ID NO:22), and LCVR CDR3 sequence QQYYSYPFT (SEQ ID NO:31), wherein the antibody or antigen-binding fragment thereof specifically binds Gal9.

In one embodiment, the isolated monoclonal antibody or antigen-binding fragment thereof comprises (a) a HCVR comprising (i) a HCVR CDR1 sequence having the amino acid sequence GYX₁FX₂X₃YTIH (SEQ ID NO:1), wherein X₁ is selected from T or E, and X₂ is selected from T or G, and X₃ is E; (ii) a HCVR CDR2 sequence having the amino acid sequence WFYPGSGSTX₄YAQKFQG (SEQ ID NO:8), wherein X₄ is selected from W or V; and (iii) a HCVR CDR3 sequence having the amino acid sequence HGGYDGFDY (SEQ ID NO:12); and (b) a LCVR comprising (i) a LCVR CDR1 sequence having the amino acid sequence KSSX₅X₆X₇LX₈X₉X₁₀X₁₁X₁₂X₁₃NX₁₄LA (SEQ ID NO:13), wherein X₅ is Q, X₆ is S, X₇ is L, X₈ is Y, X₉ is selected from S or P, X₁₀ is N, X₁₁ is N, X₁₂ is Q, X₁₃ is K; and X₁₄ is Y; (ii) a LCVR CDR2 sequence having the amino acid sequence WX₁₅SX₁₆RX₁₇X₁₈, wherein X₁₅ is A, X₁₆ is T, X₁₇ is G, and X₁₈ is selected from S or E, and (iii) a LCVR CDR3 sequence having the amino acid sequence QQYYX₁₉X₂₀PFT (SEQ ID NO:30), wherein X₁₉ is S, and X₂₀ is selected from Y or F, wherein the antibody or antigen-binding fragment thereof specifically binds Gal9.

In specific embodiments therein, the invention provides an isolated monoclonal antibody or an antigen-binding fragment thereof comprising (A) a HCVR, including a HCVR CDR1 sequence of SEQ ID NO:3, a HCVR CDR2 sequence of SEQ ID NO:10, and a HCVR CDR3 sequence of SEQ ID NO:12; and, a LCVR, including a LCVR CDR1 sequence of SEQ ID NO:15, a LCVR CDR2 sequence of SEQ ID NO:23, and a LCVR CDR3 sequence of SEQ ID NO:32; or (B) a HCVR, including a HCVR CDR1 sequence of SEQ ID NO:4, a HCVR CDR2 sequence of SEQ ID NO:11, and a HCVR CDR3 sequence of SEQ ID NO:12; and, a light chain variable region (LCVR), including a LCVR CDR1 sequence of SEQ ID NO:14, a LCVR CDR2 sequence of SEQ ID NO:24, and a LCVR CDR3 sequence of SEQ ID NO:31, wherein the antibody or antigen-binding fragment thereof specifically binds Gal9.

In one embodiment, the isolated monoclonal antibody or an antigen-binding fragment thereof comprises (a) a HCVR, comprising (i) a HCVR CDR1 sequence having the amino acid sequence GYX₁FX₂X₃YTIH (SEQ ID NO: 1), wherein X₁ is selected from T, P, or A; X₂ is selected from T, S, or G; and X₃ is selected from S or D; (ii) a HCVR CDR2 sequence having the amino acid sequence WFYPGSGSTX₄YAQKFQG (SEQ ID NO:8), wherein X₄ is E; and (iii) a HCVR CDR3 sequence having the amino acid sequence HGGYDGFDY (SEQ ID NO:12); and (b) a LCVR comprising: (i) a LCVR CDR1 sequence having the amino acid sequence KSSX₅X₆X₇LX₈X₉X₁₀X₁₁X₁₂X₁₃NX₁₄LA (SEQ ID NO:13), wherein X₅ is selected from Q or R; X₆ is selected from S or N; X₇ is selected from L, V, or I; X₈ is selected from Y or W; X₉ is S, X₁₀ is selected from S, P, or A; X₁₁ is selected from N or H; X₁₂ is N or Y; X₁₃ is K or R; and X₁₄ is Y or H; (ii) a LCVR CDR2 sequence having the amino acid sequence WX₁₅SX₁₆RX₁₇X₁₈, wherein X₁₅ is selected from A or G; X₁₆ is selected from T, N, M, or A; X₁₇ is selected from G or E; and X₁₈ is selected from S, Y, P, or T; and (iii) a LCVR CDR3 sequence having the amino acid sequence QQYYX₁₉X₂₀PFT (SEQ ID NO:30), wherein X₁₉ is F, and X₂₀ is Y; wherein the antibody or antigen-binding fragment thereof specifically binds Gal9.

In specific embodiments therein, the invention provides an isolated monoclonal antibody or an antigen-binding fragment thereof comprising (C) a HCVR, comprising a HCVR CDR1 sequence of SEQ ID NO:5, a HCVR CDR2 sequence of SEQ ID NO:9, and a HCVR CDR3 sequence of SEQ ID NO:12; and, a light chain variable region (LCVR), comprising a LCVR CDR1 sequence of SEQ ID NO: 16, a LCVR CDR2 sequence of SEQ ID NO:25, and a LCVR CDR3 sequence of SEQ ID NO:33; (D) a HCVR, comprising a HCVR CDR1 sequence of SEQ ID NO:6, a HCVR CDR2 sequence of SEQ ID NO:9, and a HCVR CDR3 sequence of SEQ ID NO:12; and a LCVR, comprising a LCVR CDR1 sequence of SEQ ID NO:17, a LCVR CDR2 sequence of SEQ ID NO:26, and a LCVR CDR3 sequence of SEQ ID NO:33, (E) a HCVR, comprising a HCVR CDR1 sequence of SEQ ID NO:7, a HCVR CDR2 sequence of SEQ ID NO:9, and a HCVR CDR3 sequence of SEQ ID NO:12; and a LCVR, comprising a LCVR CDR1 sequence of SEQ ID NO:18, a LCVR CDR2 sequence of SEQ ID NO:27, and a LCVR CDR3 sequence of SEQ ID NO:33, (F) a HCVR, comprising a HCVR CDR1 sequence of SEQ ID NO:7, a HCVR CDR2 sequence of SEQ ID NO:9, and a HCVR CDR3 sequence of SEQ ID NO:12; and a LCVR, comprising a LCVR CDR1 sequence of SEQ ID NO:19, a LCVR CDR2 sequence of SEQ ID NO:28, and a LCVR CDR3 sequence of SEQ ID NO:33, or (G) a HCVR, comprising a HCVR CDR1 sequence of SEQ ID NO:7, a HCVR CDR2 sequence of SEQ ID NO:9, and a HCVR CDR3 sequence of SEQ ID NO:12; and a LCVR, comprising a LCVR CDR1 sequence of SEQ ID NO:20, a LCVR CDR2 sequence of SEQ ID NO:29, and a LCVR CDR3 sequence of SEQ ID NO:33, wherein the antibody or antigen-binding fragment thereof specifically binds Gal9.

In another embodiment, the invention provides an isolated monoclonal antibody or an antigen-binding fragment thereof comprising (a) a HCVR, comprising (i) a HCVR CDR1 sequence having the amino acid sequence GYX₁FX₂X₃YTIH (SEQ ID NO:1), wherein X₁ is selected from E or P; X₂ is selected from T or S; and X₃ selected from E or S; (ii) a HCVR CDR2 sequence having the amino acid sequence WF YPGSGSTX₄YAQKFQG (SEQ ID NO:8), wherein X₄ is selected from W or E; and (iii) a HCVR CDR3 sequence having the amino acid sequence HGGYDGFDY (SEQ ID NO:12), and (b) a light chain variable region (LCVR), comprising: (i) a LCVR CDR1 sequence having the amino acid sequence KSSX₅X₆X₇LX₈X₉X₁₀X₁₁X₁₂X₁₃NX₁₄LA (SEQ ID NO:13), wherein X₅ is Q, X₆ is selected from S or N, X₇ is selected from L or V, X₈ is selected from Y or W, X₉ is selected from P or S, X₁₀ is selected from N or S, X₁₁ is N, X₁₂ is selected from Q or N, X₁₃ is K; and X₁₄ is Y; (ii) a LCVR CDR2 sequence having the amino acid sequence WX₁₅SX₁₆RX₁₇X₁₈, wherein X₁₅ is selected from A or G; X₁₆ is selected from T or N; X₁₇ is selected from G or E, and X₁₈ is S; and (iii) a LCVR CDR3 sequence having the amino acid sequence QQYYX₁₉X₂₀PFT (SEQ ID NO:30), wherein X₁₉ is selected from S or F; and X₂₀ is selected from Y or F, wherein the antibody or antigen-binding fragment thereof specifically binds Gal9.

In specific embodiments therein, the invention provides an isolated monoclonal antibody or an antigen-binding fragment thereof comprising (A) a HCVR, including a HCVR CDR1 sequence of SEQ ID NO:3, a HCVR CDR2 sequence of SEQ ID NO:10, and a HCVR CDR3 sequence of SEQ ID NO:12; and, a LCVR, including a LCVR CDR1 sequence of SEQ ID NO:15, a LCVR CDR2 sequence of SEQ ID NO:23, and a LCVR CDR3 sequence of SEQ ID NO:32; or (C) a HCVR, comprising a HCVR CDR1 sequence of SEQ ID NO:5, a HCVR CDR2 sequence of SEQ ID NO:9, and a HCVR CDR3 sequence of SEQ ID NO:12; and, a light chain variable region (LCVR), comprising a LCVR CDR1 sequence of SEQ ID NO:16, a LCVR CDR2 sequence of SEQ ID NO:25, and a LCVR CDR3 sequence of SEQ ID NO:33, wherein the antibody or antigen-binding fragment thereof specifically binds Gal9.

In one aspect, the HCVR sequence of the antibody or antigen-binding fragment thereof of (A), (B), (C), (D), (E), (F), or (G), further includes a HFR3 sequence of SEQ ID NO:68, and optionally further includes a HFR1 sequence of SEQ ID NO:66, a HFR2 sequence of SEQ ID NO:67, and/or a HFR4 sequence of SEQ ID NO:71. In another aspect, the HCVR sequence of the antibody or antigen-binding fragment thereof of (A), (B), (C), (D), (E), (F), or (G) further includes a HFR3 sequence of SEQ ID NO:69, and optionally further includes a HFR1 sequence of SEQ ID NO:66, a HFR2 sequence of SEQ ID NO:67, and/or a HFR4 sequence of SEQ ID NO:71. In another aspect, the HCVR sequence of the antibody or antigen-binding fragment thereof of (A), (B), (C), (D), (E), (F), or (G) further includes a HFR3 sequence of SEQ ID NO:70, and optionally further includes a HFR1 sequence of SEQ ID NO:66, a HFR2 sequence of SEQ ID NO:67, and/or a HFR4 sequence of SEQ ID NO:71. In another aspect, the HCVR sequence of the antibody or antigen-binding fragment thereof of (A), (B), (C), (D), (E), (F), or (G) further includes a HFR3 sequence of SEQ ID NO:76, and optionally further includes a HFR1 sequence of SEQ ID NO:66, a HFR2 sequence of SEQ ID NO:67, and/or a HFR4 sequence of SEQ ID NO:71. In another aspect, the HCVR sequence of the antibody or antigen-binding fragment thereof of (A), (B), (C), (D), (E), (F), or (G) further includes a HFR3 sequence of SEQ ID NO:77, and optionally further includes a HFR1 sequence of SEQ ID NO:66, a HFR2 sequence of SEQ ID NO:67, and/or a HFR4 sequence of SEQ ID NO:71. In one aspect, the LCVR sequence of the antibody or antigen-binding fragment thereof of (A), (B), (C), (D), (E), (F), or (G) optionally further includes a LFR1 sequence of SEQ ID NO:72, a LFR2 sequence of SEQ ID NO:73, a LFR3 sequence of SEQ ID NO:74, and/or a LFR4 sequence of SEQ ID NO:75.

In another aspect, the HCVR sequence is SEQ ID NO:39 and/or the LCVR sequence is SEQ ID NO:41; or the HCVR sequence is SEQ ID NO:43 and/or the LCVR sequence is SEQ ID NO:45; or the HCVR sequence is SEQ ID NO:47 and/or, the LCVR sequence is SEQ ID NO: 49; or the HCVR sequence is SEQ ID NO:51 and/or the LCVR sequence is SEQ ID NO:53; or the HCVR sequence is SEQ ID NO:55 and/or the LCVR sequence is SEQ ID NO:57; or the HCVR sequence is SEQ ID NO:59 and/or the LCVR sequence is SEQ ID NO:61; or the HCVR sequence is SEQ ID NO:63 and/or the LCVR sequence is SEQ ID NO:65. In some aspects, the antibody includes: the HCVR sequence of SEQ ID NO:39 and the LCVR sequence of SEQ ID NO:41; or the HCVR sequence of SEQ ID NO:43 and the LCVR sequence of SEQ ID NO:45; or the HCVR sequence of SEQ ID NO:47 and the LCVR sequence of SEQ ID NO:49; or the HCVR sequence of SEQ ID NO:51 and the LCVR sequence of SEQ ID NO:53; or the HCVR sequence of SEQ ID NO:55 and the LCVR sequence of SEQ ID NO:57; or the HCVR sequence of SEQ ID NO:59 and the LCVR sequence of SEQ ID NO:61; or the HCVR sequence of SEQ ID NO:63 and the LCVR sequence of SEQ ID NO:65.

In one aspect, the antibody is a humanized antibody. In another aspect, the antigen-binding fragment thereof is a Fab, Fab′, F(ab′)₂, F_{d}, single chain Fv or scFv, disulfide linked Fᵥ, V-NAR domain, IgNar, intrabody, IgGACH₂, minibody, F(ab′)₃, tetrabody, triabody, diabody, single-domain antibody, DVD-Ig, Fcab, mAb₂, (scFv)₂, tandem scFv, DART®, TandAb, nanobody or scFv-Fc. In other aspects, the monoclonal antibody or antigen-binding fragment thereof binds human Gal9 with a K_{d} of less than about 5 nM, 2 nM, 1 nM, 0.5 nM, 0.4 nM, 0.3 nM, 0.2 nM, 0.1 nM, 0.05 nM or 0.01 nM. In one aspect, the antibody binds to Gal9 and inhibits Gal9 binding to a Gal9 receptor (e.g., TIM3 or CD44). In another aspect, the antibody binds to Gal9 and does not inhibit Gal9 binding to a Gal9 receptor (e.g., TIM3 or CD44). In one aspect, the antibody reverses Gal9-induced Th1 apoptosis of T cells (such as CD4⁺ T cells). In another aspect, the antibody suppresses Gal9-induced Treg expansion.

In another embodiment, the invention provides a method of treating cancer in a subject in need thereof including administering to the subject an effective amount of any one of the isolated monoclonal antibodies or antigen-binding fragments described herein, thereby treating cancer in the subject.

In one aspect, the cancer is a hematological cancer or a solid tumor. In some aspects, the hematological cancer is selected from the group consisting of acute lymphoblastic leukemia (ALL), acute myeloid leukemia (AML), chronic lymphocytic leukemia (CLL), chronic myelogenous leukemia (CMIL), and hairy cell leukemia; AIDS-related lymphoma, cutaneous T-cell lymphoma, Hodgkin lymphoma, mycosis fungoides, non-Hodgkin lymphoma, primary central nervous system lymphoma, Sézary syndrome, cutaneous T-Cell lymphoma, Waldenström macroglobulinemia, diffuse large B-Cell Lymphoma (DLBCL), and multiple myeloma. In other aspects, the solid tumor is selected from the group consisting of breast cancer, head and neck cancer, lung cancer, melanoma, uveal melanoma, colorectal cancer, renal carcinoma, urothelial cancer, ovarian cancer, endometrial cancer, liver cancer, pancreatic cancer, cholangiocarcinoma, gastric cancer, gastroesophageal cancer, esophageal cancer, cervical cancer, head and neck squamous cancer, and prostate cancer. In one aspect, the method further includes administering to the patient a chemotherapeutic agent, an anti-angiogenesis agent, a growth inhibitory agent, an immune-oncology agent, a checkpoint inhibitor, and/or an anti-neoplastic composition. In some aspects, the checkpoint inhibitor is an anti-PD-1. In other aspects, the immune-oncology agent is an anti-glucocorticoid-induced tumor necrosis factor receptor family-related protein (GITR) antibody.

In an additional embodiment, the invention provides a polynucleotide encoding the heavy chain or the light chain or the antigen-binding portion thereof of any one of the antibodies or antigen-binding fragments thereof described herein.

In one embodiment, the invention provides a vector including the polynucleotide described herein, wherein the vector is an expression vector selected from the group consisting of a mammalian expression vector, a yeast expression vector, an insect expression vector, and a bacterial expression vector.

In another embodiment, the invention provides a method of rescuing or promoting effector T cell proliferation, enhancing effector T cell activity, and/or of identifying and treating cancer in a subject including: (i) determining a level of Gal9 in a sample from the subject, and (ii) administering to a subject having a level Gal9 higher than a reference level of Gal9 an effective amount of any one of the isolated monoclonal antibodies or antigen-binding fragments thereof described herein, thereby rescuing or promoting effector T cells proliferation, enhancing effector T cell activity, and/or identifying and treating a subject.

In one aspect, the subject is diagnosed with cancer, is at risk of developing cancer or has cancer relapse. In another aspect, the reference level of Gal9 is a level of Gal9 measured in a sample from a healthy or a control individual. In some aspects, determining includes comparing the level of Gal9 in the sample to the reference level. In one aspect, the cancer is a hematological cancer or a solid tumor. In some aspects, the hematological cancer is selected from the group consisting of ALL, AML, CLL, CML, and hairy cell leukemia; AIDS-related lymphoma, cutaneous T-cell lymphoma, Hodgkin lymphoma, mycosis fungoides, non-Hodgkin lymphoma, primary central nervous system lymphoma, Sézary syndrome, cutaneous T-Cell lymphoma, Waldenström macroglobulinemia, DLBCL, and multiple myeloma. In other aspects, the solid tumor is selected from the group consisting of breast cancer, head and neck cancer, lung cancer, melanoma, uveal melanoma, colorectal cancer, renal carcinoma, urothelial cancer, ovarian cancer, endometrial cancer, liver cancer, pancreatic cancer, cholangiocarcinoma, gastric cancer, gastroesophageal cancer, esophageal cancer, cervical cancer, head and neck squamous cancer, and prostate cancer. In one aspect, the sample is a blood sample, a plasma sample, a serum sample, a tumor biopsy sample, or a bone-marrow (BM)-derived mononuclear cell (MNC) sample. In one aspect, the method further includes administering to the patient a chemotherapeutic agent, an anti-angiogenesis agent, a growth inhibitory agent, an immune-oncology agent, a checkpoint inhibitor, and/or an anti-neoplastic composition. In some aspects, the checkpoint inhibitor is an anti-PD-1 antibody. In other aspects, the immune-oncology agent is an anti-GITR antibody.

### BRIEF DESCRIPTION OF THE DRAWINGS

FIGS. 1A-1D show sequence alignments. FIG. 1A shows the alignments of the sequences of the heavy chains of antibody A (Ab A), antibody 001 (Ab 001) and antibody 002 (Ab 002) anti-Gal9 antibodies. FIG. 1B shows the alignments of the sequences of the light chains of Ab A, Ab 001 and Ab 002 anti-Gal9 antibodies. FIG. 1C shows the alignments of the sequences of the heavy chains of antibody A (Ab A), antibody 003 (Ab 003), antibody 004 (Ab 004), antibody 005 (Ab 005), antibody 006 (Ab 006) and antibody 007 (Ab 007) anti-Gal9 antibodies. FIG. 1D shows the alignments of the sequences of the light chains of Ab A, Ab 003, Ab 004, Ab 005, Ab 006, and Ab 007 anti-Gal9 antibodies.

FIGS. 2A-2D show the effect of anti-Gal9 antibodies on Gal9-induced apoptosis. FIG. 2A shows the effect of the Ab A anti-Gal9 antibody on T-cell apoptosis. FIG. 2B shows the effect of the affinity matured Ab 001 anti-Gal9 antibody on T-cell apoptosis. FIG. 2C shows the effect of Ab 001 on Gal9 induced CD4+ T cell apoptosis. FIG. 2D shows the effect of Ab 001 on Gal9 induced CD8+ T cell apoptosis.

FIG. 3 shows the comparative effect of Ab 001 and Ab 003 on TIM3 dimerization.

### DETAILED DESCRIPTION OF THE INVENTION

Aspects of the invention described herein relate to antibodies directed against Gal9 and the use thereof for the treatment of diseases associated with the suppressor activity of regulatory T lymphocytes (Tregs).

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

Provided herein are affinity matured antibodies that specifically bind to Gal9. Affinity maturation is a process used to generate increased affinity, avidity, and anti-antigen activity from an antibody (See, e.g., Teixeira et al., mAbs 14(1) e2115200, 2022).

Affinity maturation was carried out on antibody HFB9-2hz11 (Ab A) disclosed in International Publication No. WO WO2021/139682. Seven antibodies were identified having substantially improved Gal9 binding affinity. These antibodies demonstrated variability in the CDR regions that retained and improved upon Gal9 binding over the parent antibody. Thus, in one aspect, the invention provides an isolated monoclonal antibody or an antigen-binding fragment thereof comprising (a) a heavy chain variable region (HCVR) comprising (i) a HCVR CDR1 sequence having the amino acid sequence GYX₁FX₂X₃YTIH (SEQ ID NO:1), wherein X₁ is selected from T, E, P, or A; X₂ is selected from T, G, or S; and X₃ is selected from E, S, or D; (ii) a HCVR CDR2 sequence having the amino acid sequence WFYPGSGSTX₄YAQKFQG (SEQ ID NO:8), wherein X₄ is selected from E, W, or V; and (iii) a HCVR CDR3 sequence having the amino acid sequence HGGYDGFDY (SEQ ID NO:12); and (b) a light chain variable region (LCVR) comprising (i) a LCVR CDR1 sequence having the amino acid sequence KSSX₅X₆X₇LX₈X₉X₁₀X₁₁X₁₂X₁₃NX₁₄LA (SEQ ID NO:13), wherein X₅ is selected from Q or R; X₆ is selected from S or N; X₇ is selected from L, V, or I; X₈ is selected from Y or W; X₉ is selected from S or P; X₁₀ is selected from N, S, P, or A; X₁₁ is selected from N or H; X₁₂ is selected from Q, N, or Y; X₁₃ is selected from K or R; and X₁₄ is selected from Y or H; (ii) a LCVR CDR2 sequence having the amino acid sequence WX₁₅SX₁₆RX₁₇X₁₈, wherein X₁₅ is selected from A or G; X₁₁ is selected from T, N, M, or A; X₁₇ is selected from G or E; and X₁₈ is selected from S, E, Y, P, or T; and (iii) a LCVR CDR3 sequence having the amino acid sequence QQYYX₁₉X₂₀PFT (SEQ ID NO:30), wherein X₁₉ is selected from S or F; and X₂₀ is selected from Y or F, with the proviso that the antibody does not comprise HCVR CDR1 sequence GYTFTEYTIH (SEQ ID NO:2), HCVR CDR2 sequence WFYPGSGSTEYAQKFQG (SEQ ID NO:9), HCVR CDR3 sequence HGGYDGFDY (SEQ ID NO:12), LCVR CDR1 sequence KSSQSLLYSNNQKNYLA (SEQ ID NO:14), LCVR CDR2 sequence WASTRGS (SEQ ID NO:22), and LCVR CDR3 sequence QQYYSYPFT (SEQ ID NO:31), wherein the antibody or antigen-binding fragment thereof specifically binds Gal9.

In one embodiment, the isolated monoclonal antibody or antigen-binding fragment thereof comprises (a) a HCVR comprising (i) a HCVR CDR1 sequence having the amino acid sequence GYX₁FX₂X₃YTIH (SEQ ID NO:1), wherein X₁ is selected from T or E, and X₂ is selected from T or G, and X₃ is E; (ii) a HCVR CDR2 sequence having the amino acid sequence WFYPGSGSTX₄YAQKFQG (SEQ ID NO:8), wherein X₄ is selected from W or V; and (iii) a HCVR CDR3 sequence having the amino acid sequence HGGYDGFDY (SEQ ID NO:12); and (b) a LCVR comprising (i) a LCVR CDR1 sequence having the amino acid sequence KSSX₅X₆X₇LX₈X₉X₁₀X₁₁X₁₂X₁₃N X₁₄LA (SEQ ID NO:13), wherein X₅ is Q, X₆ is S, X₇ is L, X₈ is Y, X₉ is selected from S or P, X₁₀ is N, X₁₁ is N, X₁₂ is Q, X₁₃ is K; and X₁₄ is Y; (ii) a LCVR CDR2 sequence having the amino acid sequence WX₁₅SX₁₆RX₁₇X₁₈, wherein X₁₅ is A, X₁₆ is T, X₁₇ is G, and X₁₈ is selected from S or E, and (iii) a LCVR CDR3 sequence having the amino acid sequence QQYYX₁₉X₂₀PFT (SEQ ID NO:30), wherein X₁₉ is S, and X₂₀ is selected from Y or F, wherein the antibody or antigen-binding fragment thereof specifically binds Gal9.

Specific embodiments therein include an isolated monoclonal antibody or an antigen-binding fragment thereof comprising (A) a HCVR, including a HCVR CDR1 sequence of SEQ ID NO:3, a HCVR CDR2 sequence of SEQ ID NO:10, and a HCVR CDR3 sequence of SEQ ID NO:12; and a LCVR, including a LCVR CDR1 sequence of SEQ ID NO:15, a LCVR CDR2 sequence of SEQ ID NO:23, and a LCVR CDR3 sequence of SEQ ID NO:32; or (B) a HCVR, including a HCVR CDR1 sequence of SEQ ID NO:4, a HCVR CDR2 sequence of SEQ ID NO:11, and a HCVR CDR3 sequence of SEQ ID NO:12; and, a light chain variable region (LCVR), including a LCVR CDR1 sequence of SEQ ID NO:14, a LCVR CDR2 sequence of SEQ ID NO:24, and a LCVR CDR3 sequence of SEQ ID NO:31, wherein the antibody or antigen-binding fragment thereof specifically binds Gal9.

In another embodiment, the isolated monoclonal antibody or an antigen-binding fragment thereof comprises (a) a HCVR, comprising (i) a HCVR CDR1 sequence having the amino acid sequence GYX₁FX₂X₃YTIH (SEQ ID NO:1), wherein X₁ is selected from T, P, or A; X₂ is selected from T, S, or G; and X₃ is selected from S or D; (ii) a HCVR CDR2 sequence having the amino acid sequence WFYPGSGSTX₄YAQKFQG (SEQ ID NO:8), wherein X₄ is E; and (iii) a HCVR CDR3 sequence having the amino acid sequence HGGYDGFDY (SEQ ID NO:12); and (b) a LCVR comprising: (i) a LCVR CDR1 sequence having the amino acid sequence KSSX₅X₆X₇LX₈X₉X₁₀X₁₁X₁₂X₁₃N X₁₄LA (SEQ ID NO:13), wherein X₅ is selected from Q or R; X₆ is selected from S or N; X₇ is selected from L, V, or I; X₈ is selected from Y or W; X₉ is S, X₁₀ is selected from S, P, or A; X₁₁ is selected from N or H; X₁₂ is N or Y; X₁₃ is K or R; and X₁₄ is Y or H; (ii) a LCVR CDR2 sequence having the amino acid sequence WX₁₅SX₁₆RX₁₇X₁₈, wherein X₁₅ is selected from A or G; X₁₆ is selected from T, N, M, or A; X₁₇ is selected from G or E; and X₁₈ is selected from S, Y, P, or T; and (iii) a LCVR CDR3 sequence having the amino acid sequence QQYYX₁₉X₂₀PFT (SEQ ID NO:30), wherein X₁₉ is F, and X₂₀ is Y; wherein the antibody or antigen-binding fragment thereof specifically binds Gal9.

Specific embodiments therein include an isolated monoclonal antibody or an antigen-binding fragment thereof comprising (C) a HCVR, comprising a HCVR CDR1 sequence of SEQ ID NO:5, a HCVR CDR2 sequence of SEQ ID NO:9, and a HCVR CDR3 sequence of SEQ ID NO:12; and, a light chain variable region (LCVR), comprising a LCVR CDR1 sequence of SEQ ID NO:16, a LCVR CDR2 sequence of SEQ ID NO:25, and a LCVR CDR3 sequence of SEQ ID NO:33; (D) a HCVR, comprising a HCVR CDR1 sequence of SEQ ID NO:6, a HCVR CDR2 sequence of SEQ ID NO:9, and a HCVR CDR3 sequence of SEQ ID NO:12; and a LCVR, comprising a LCVR CDR1 sequence of SEQ ID NO:17, a LCVR CDR2 sequence of SEQ ID NO:26, and a LCVR CDR3 sequence of SEQ ID NO:33, (E) a HCVR, comprising a HCVR CDR1 sequence of SEQ ID NO:7, a HCVR CDR2 sequence of SEQ ID NO:9, and a HCVR CDR3 sequence of SEQ ID NO:12; and a LCVR, comprising a LCVR CDR1 sequence of SEQ ID NO:18, a LCVR CDR2 sequence of SEQ ID NO:27, and a LCVR CDR3 sequence of SEQ ID NO:33, (F) a HCVR, comprising a HCVR CDR1 sequence of SEQ ID NO:7, a HCVR CDR2 sequence of SEQ ID NO:9, and a HCVR CDR3 sequence of SEQ ID NO: 12; and a LCVR, comprising a LCVR CDR1 sequence of SEQ ID NO:19, a LCVR CDR2 sequence of SEQ ID NO:28, and a LCVR CDR3 sequence of SEQ ID NO:33, or (G) a HCVR, comprising a HCVR CDR1 sequence of SEQ ID NO:7, a HCVR CDR2 sequence of SEQ ID NO:9, and a HCVR CDR3 sequence of SEQ ID NO:12; and a LCVR, comprising a LCVR CDR1 sequence of SEQ ID NO:20, a LCVR CDR2 sequence of SEQ ID NO:29, and a LCVR CDR3 sequence of SEQ ID NO:33, wherein the antibody or antigen-binding fragment thereof specifically binds Gal9.

In another embodiment, the invention provides an isolated monoclonal antibody or an antigen-binding fragment thereof comprising (a) a HCVR, comprising (i) a HCVR CDR1 sequence having the amino acid sequence GYX₁FX₂X₃YTIH (SEQ ID NO:1), wherein X₁ is selected from E or P; X₂ is selected from T or S; and X₃ selected from E or S; (ii) a HCVR CDR2 sequence having the amino acid sequence WF YPGSGSTX₄YAQKFQG (SEQ ID NO:8), wherein X₄ is selected from W or E; and (iii) a HCVR CDR3 sequence having the amino acid sequence HGGYDGFDY (SEQ ID NO:12), and (b) a light chain variable region (LCVR), comprising: (i) a LCVR CDR1 sequence having the amino acid sequence KSSX₅X₆X₇LX₈X₉X₁₀X₁₁X₁₂X₁₃NX₁₄LA (SEQ ID NO:13), wherein X₅ is Q, X₆ is selected from S or N, X₇ is selected from L or V, X₈ is selected from Y or W, X₉ is selected from P or S, X₁₀ is selected from N or S, X₁₁ is N, X₁₂ is selected from Q or N, X₁₃ is K; and X₁₄ is Y; (ii) a LCVR CDR2 sequence having the amino acid sequence WX₁₅SX₁₆RX₁₇X₁₈, wherein X₁₅ is selected from A or G; X₁₆ is selected from T or N; X₁₇ is selected from G or E, and X₁₈ is S; and (iii) a LCVR CDR3 sequence having the amino acid sequence QQYYX₁₉X₂₀PFT (SEQ ID NO:30), wherein X₁₉ is selected from S or F; and X₂₀ is selected from Y or F, wherein the antibody or antigen-binding fragment thereof specifically binds Gal9.

Specific embodiments therein include an isolated monoclonal antibody or an antigen-binding fragment thereof comprising (A) a HCVR, including a HCVR CDR1 sequence of SEQ ID NO:3, a HCVR CDR2 sequence of SEQ ID NO:10, and a HCVR CDR3 sequence of SEQ ID NO: 12; and a LCVR, including a LCVR CDR1 sequence of SEQ ID NO:15, a LCVR CDR2 sequence of SEQ ID NO:23, and a LCVR CDR3 sequence of SEQ ID NO:32; or (C) a HCVR, comprising a HCVR CDR1 sequence of SEQ ID NO:5, a HCVR CDR2 sequence of SEQ ID NO:9, and a HCVR CDR3 sequence of SEQ ID NO:12; and, a light chain variable region (LCVR), comprising a LCVR CDR1 sequence of SEQ ID NO:16, a LCVR CDR2 sequence of SEQ ID NO:25, and a LCVR CDR3 sequence of SEQ ID NO:33, wherein the antibody or antigen-binding fragment thereof specifically binds Gal9.

The terms “antibodies,” “abs,” “immunoglobulins,” or “Igs” in the broadest sense, refer to glycoproteins having the same structural characteristics (i.e., molecules that contain an antigen binding site that immunospecifically binds an antigen) and encompass various antibody structures, including natural or artificial, mono- or polyvalent antibodies including but not limited to monoclonal antibodies (including chimeric monoclonal antibodies, humanized monoclonal antibodies, and human monoclonal antibodies, particularly humanized monoclonal antibodies), polyclonal antibodies, single chain antibodies, antibody fragments, and multispecific antibodies (e.g., bispecific antibodies). While antibodies exhibit binding specificity to a specific antigen, immunoglobulins include both antibodies and other antibody-like molecules which lack antigen specificity. “Antibody,” encompasses any polypeptide comprising an antigen-binding site regardless of the source, species of origin, method of production, and characteristics. The term “antibody” may also broadly refer to a molecule comprising complementarity determining region (CDR) 1, CDR2, and CDR3 of a heavy chain and CDR1, CDR2, and CDR3 of a light chain, wherein the molecule is capable of binding to an antigen.

In some embodiments, an antibody comprises a heavy chain variable region (HCVR) and a light chain variable region (LCVR). In some embodiments, an antibody comprises at least one heavy chain (HC) comprising a heavy chain variable region and at least a portion of a heavy chain constant region, and at least one light chain (LC) comprising a light chain variable region and at least a portion of a light chain constant region. In some embodiments, an antibody comprises two heavy chains, wherein each heavy chain comprises a heavy chain variable region and at least a portion of a heavy chain constant region, and two light chains, wherein each light chain comprises a light chain variable region and at least a portion of a light chain constant region.

“Native antibodies” and “intact immunoglobulins”, or the like, are usually heterotetrameric glycoproteins of about 150,000 daltons, composed of two identical light (L) chains and two identical heavy (H) chains. The light chains from any vertebrate species can be assigned to one of two clearly distinct types, called kappa (κ) and lambda (λ), based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes). For example, IgG antibodies include, but are not limited to, IgG1 (comprising a γ1 constant region), IgG2 (comprising a γ2 constant region), IgG3 (comprising a γ3 constant region), and IgG4 (comprising a γ4 constant region) antibodies; IgA antibodies include, but are not limited to, IgA1 (comprising an α1 constant region) and IgA2 (comprising an α2 constant region) antibodies; and IgM antibodies include, but are not limited to, IgM1 (comprising an μ1 constant region) and IgM2 (comprising an μ2 constant region). The heavy-chain constant domains that correspond to the different classes of immunoglobulins are called α, δ, ε, γ, and μ, respectively. The subunit structures and three-dimensional configurations of different classes of immunoglobulins are well known.

The intact antibody may have one or more “effector functions” which refer to those biological activities attributable to the Fc region (a native sequence Fc region or amino acid sequence variant Fc region or any other modified Fc region) of an antibody. Examples of antibody effector functions include Clq binding; complement dependent cytotoxicity; Fc receptor binding; antibody-dependent cell-mediated cytotoxicity (ADCC); phagocytosis; down regulation of cell surface receptors (e.g., B cell receptor (BCR)); and cross-presentation of antigens by antigen presenting cells or dendritic cells.

Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light-chain variable domain is aligned with the variable domain of the heavy chain. Particular amino acid residues are believed to form an interface between the light- and heavy-chain variable domains. Each variable region includes three segments called complementarity-determining regions (CDRs) or hypervariable regions and more highly conserved portions of variable domains are called the framework regions (FR). The variable domains of heavy and light chains each include four FR regions, largely adopting a j-sheet configuration, connected by three CDRs, which form loops connecting, and in some cases forming part of the j-sheet structure. The CDRs in each chain are held together in close proximity by the FRs and, with the CDRs from the other chain, contribute to the formation of the antigen-binding site of antibodies (see Kabat et al., NIH Publ. No. 91-3242, Vol. I, pages 647-669 [1991]). The constant domains are not involved directly in binding an antibody to an antigen, but exhibit various effector functions, such as participation of the antibody in antibody dependent cellular cytotoxicity.

The term “heavy chain variable region (HCVR)” as used herein refers to, at a minimum, a region comprising heavy chain CDR1 (CDR-H1), framework 2 (HFR2), CDR2 (CDR-H2), FR3 (HFR3), and CDR3 (CDR-H3). In some embodiments, a heavy chain variable region also comprises at least a portion (e.g., the whole) of an FR1 (HFR1), which is N-terminal to CDR-H1, and/or at least a portion (e.g., the whole) of an FR4 (HFR4), which is C-terminal to CDR-H3.

The term “heavy chain constant region” as used herein refers to a region comprising at least three heavy chain constant domains, CH1, CH2, and CH3. Non-limiting exemplary heavy chain constant regions include γ, δ, and α. Non-limiting exemplary heavy chain constant regions also include ε and μ. Each heavy constant region corresponds to an antibody isotype. For example, an antibody comprising a γ constant region is an IgG antibody, an antibody comprising a δ constant region is an IgD antibody, an antibody comprising an α constant region is an IgA antibody, an antibody comprising an ε constant region is an IgE antibody, and an antibody comprising an μ constant region is an IgM antibody.

The term “light chain variable region (LCVR)” as used herein refers to a region comprising light chain CDR1 (CDR-L1), framework (FR) 2 (LFR2), CDR2 (CDR-L2), FR3 (LFR3), and CDR3 (CDR-L3). In some embodiments, a light chain variable region also comprises at least a portion (e.g., the whole) of an FR1 (LFR1), which is N-terminal to CDR-L1, and/or at least a portion (e.g., the whole) of an FR4 (LFR4), which is C-terminal to CDR-L3.

The term “light chain constant region” as used herein refers to a region comprising a light chain constant domain, CL. Non-limiting exemplary light chain constant regions include λ and κ.

The term “light chain” as used herein refers to a polypeptide comprising at least a light chain variable region, with or without a leader sequence. In some embodiments, a light chain comprises at least a portion of a light chain constant region. The term “full-length light chain” as used herein refers to a polypeptide comprising a light chain variable region and a light chain constant region, with or without a leader sequence.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which typically include different antibodies directed against different determinants (epitopes), each monoclonal antibody is directed against a single determinant on the antigen. In addition to their specificity, monoclonal antibodies are advantageous in that they are uncontaminated by other immunoglobulins. The modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), from yeast display, and from transgenic animal, for example.

The terms “amino acid” and “naturally occurring amino acid” in the broadest sense, refer to alpha-amino acids that are organic compounds comprising both amino and carboxylic acid functional groups, from which proteins are created. Each alpha-amino acid contains a central carbon atom linked to an amino group, a carboxylic acid, a hydrogen, and an “R” group. This “R” group varies between each amino acid and is the defining difference that differentiates each. Twenty amino acids are encoded by the eukaryotic genetic code and are naturally incorporated into polypeptide chains during the process of protein construction and are therefore referred to as “naturally occurring” amino acids. Other non-naturally occurring amino acids are known which are either not found in proteins or are not produced naturally within eukaryotic cells by standard cellular machinery (e.g., synthesis from intermediates created during glycolysis). The twenty naturally occurring amino acids are classified by the chemical properties of their varying “R” group side chains. These properties include either positively or negatively charged side chains, polar uncharged side chain, hydrophobic side chain and the special case side chains of glycine, cysteine and proline which contain side chains that are not easily classified by the above properties.

The 20 naturally occurring amino acids include: Alanine (Ala, A), Arginine (Arg, R), Asparagine (Asn, N), Aspartate (Asp, D), Cysteine (Cys, C), Glutamine (Gln, Q), Glutamate (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y) and Valine (Val, V).

Among the 20 naturally occurring amino acids, five have a side chain which can be charged. At pH=7, three are positively charged: lysine (Lys, K), arginine (Arg, R) and histidine (His, H) (basic side chains); and two are negatively charged: aspartic acid (Asp, D) and glutamic acid (Glu, E) (acidic side chains). The polar, uncharged amino acids include serine (Ser, S), threonine (Thr, T), asparagine (Asn, N) and glutamine (Gln, Q), which readily form hydrogen bonds with water and other amino acids. They do not ionize in normal conditions, though a prominent exception being the catalytic serine in serine proteases. The nonpolar or hydrophobic amino acids are alanine (Ala, A), valine (Val, V), isoleucine (Ile, I), leucine (Leu, L), methionine (Met, M), Phenylalanine (Phe, F), Tryptophan (Trp, W) and tyrosine (Tyr, Y), and their interactions are the primary driving force behind the processes that fold proteins into their functional three-dimensional structures. None of these amino acids' side chains ionize easily and therefore do not have pKas, except for tyrosine wherein the hydroxyl of tyrosine can deprotonate at high pH forming the negatively charged phenolate. Because of this one could place tyrosine into the polar, uncharged amino acid category, but its very low solubility in water matches the characteristics of hydrophobic amino acids well.

Several side chains are not described well by the charged, polar, and hydrophobic categories. Glycine (Gly, G) could be considered a polar amino acid since its small size means that its solubility is largely determined by the amino and carboxylate groups. However, the lack of any side chain provides glycine with a unique flexibility among amino acids with large ramifications to protein folding. Cysteine (Cys, C) can also form hydrogen bonds readily, which would place it in the polar amino acid category, though it can often be found in protein structures forming covalent bonds with other cysteines called disulphide bonds. These bonds influence the folding and stability of proteins and are essential in the formation of antibodies. Proline (Pro, P) has an alkyl side chain and could be considered hydrophobic, but because the side chain joins back onto the alpha amino group it becomes particularly inflexible when incorporated into proteins. Like glycine, this influences protein structure in a way unique among amino acids. G, C and P are generally classified as “special case side chain” amino acids.

The antibodies or antigen-binding fragments thereof described herein specifically bind Gal9.

Galectins, members of an animal lectin family, are defined by shared consensus amino acid sequences which confer specific binding to β-galactoside containing glycoconjugates. Galectin families are ubiquitously expressed from lower organisms, such as nematodes and sponges, to higher mammalian species, such as humans. The presence of such proteins across many species coupled with the highly conserved amino acid residues which are critical for ligand recognition in the carbohydrate recognition domain (CRD) suggests that galectins are involved in critical, conserved biological processes. Galectin CRDs, which typically consist of approximately 130 amino acid residues tightly folded into a sandwich structure of five- and six-stranded β-sheets, recognize the basic structure of N-acetyllactosamine (LacNAc). Fifteen members of the mammalian galectin family have been identified to date. Hirabayashi & Kasai proposed designating galectin subfamilies as proto-type, chimera-type, or tandem repeat-type, based on their domain organization. Proto-type galectins (galectins-1, 2, 5, 7, 10, 11, 13, 14, and 15) contain a single CRD with a short N-terminal sequence, while tandem-repeat-type galectins (galectins-4, 6, 8, 9, and 12) include two non-identical CRDs joined by a short linker peptide sequence. The single chimera-type galectin (galectin-3) has one CRD with an extended N terminus containing several repeats of a proline-tyrosine-glycine-rich motif. Gal9, a tandem-repeat-type galectin, was originally isolated from mouse embryonic kidney cells and later found to be widely distributed throughout rat and mouse tissues. In contrast, expression of human Gal9 is restricted to peripheral blood leukocytes and lymphatic tissues. It has been reported that the potent eosinophil chemoattractant ecalectin, which was originally cloned from a human T cell line, is identical with human Gal9. Several isoforms of mammalian Gal9 exist, each of which has a linker of various length.

Gal9 exhibits a variety of biological functions, including cell aggregation, eosinophil chemoattraction, and apoptosis of murine thymocytes and T cells and human melanoma cells. Mouse Gal9 induces thymocyte apoptosis in a lactose-inhibitable manner. The chemoattractant activity of Gal9 depends on its carbohydrate-binding activity and requires both CRDs. Thus, the physiological function of Gal9 likely depends on its carbohydrate recognition ability. Two physiological targets for mouse Gal9 have been reported, Tim-3 and glucose transporter (GLUT)-2. Tim-3 is specifically expressed on the surface of T helper type 1 (Th1) cells. Gal9 recognizes the carbohydrate(s) covalently bound to Tim-3; the Gal9-Tim3 pathway induces cell death in Th1 cells suggesting that Gal9 plays a crucial role in down-regulating Th1 responses.

Gal9, however, also interacts on the extracellular surface with GLUT-2, a glucose transporter expressed on the surface of pancreatic R cells that is essential for glucose-stimulated insulin secretion. The recognition of GLUT-2 by Gal9 through recognition of the carbohydrate moiety is required for the residency of GLUT-2 on the cell surface. Loss of glycosylation or the addition of glycans attenuates the half-life of GLUT-2 on the cell surface and elicits receptor endocytosis with redistribution into endosomes and lysosomes.

Although the exact target carbohydrate structures recognized by Gal9 in vivo have not been identified, in vitro analyses have demonstrated that human Gal9 has a high affinity for multiple oligosaccharides containing β-galactosides. In vitro analyses revealed that the N-terminal CRD (NCRD) and C-terminal CRD (CCRD) of human Gal9 have different oligosaccharide binding affinities. In comparison to the CCRD of human Gal9, the NCRD exhibits a striking affinity for complex glycoconjugates, such as Forssman pentasaccharide and polymerized N-acetyllactosamine.

As the biological activity of human Gal9 will depend on the ligand specificity of each CRD and the subsequent multivalent binding conferred by two CRDs, structural analysis of the specific carbohydrate recognition properties of the human Gal9 NCRD should provide insight into the physiologic function of Gal9.

In one aspect, the HCVR sequence of the antibody or antigen-binding fragment thereof of (A), (B), (C), (D), (E), (F), or (G), further includes a HFR3 sequence of SEQ ID NO:68, and optionally further includes a HFR1 sequence of SEQ ID NO:66, a HFR2 sequence of SEQ ID NO:67, and/or a HFR4 sequence of SEQ ID NO:71. In another aspect, the HCVR sequence of the antibody or antigen-binding fragment thereof of (A), (B), (C), (D), (E), (F), or (G) further includes a HFR3 sequence of SEQ ID NO:69, and optionally further includes a HFR1 sequence of SEQ ID NO:66, a HFR2 sequence of SEQ ID NO:67, and/or a HFR4 sequence of SEQ ID NO:71. In another aspect, the HCVR sequence of the antibody or antigen-binding fragment thereof of (A), (B), (C), (D), (E), (F), or (G) further includes a HFR3 sequence of SEQ ID NO:70, and optionally further includes a HFR1 sequence of SEQ ID NO:66, a HFR2 sequence of SEQ ID NO:67, and/or a HFR4 sequence of SEQ ID NO:71. In another aspect, the HCVR sequence of the antibody or antigen-binding fragment thereof of (A), (B), (C), (D), (E), (F), or (G) further includes a HFR3 sequence of SEQ ID NO:76, and optionally further includes a HFR1 sequence of SEQ ID NO:66, a HFR2 sequence of SEQ ID NO:67, and/or a HFR4 sequence of SEQ ID NO:71. In another aspect, the HCVR sequence of the antibody or antigen-binding fragment thereof of (A), (B), (C), (D), (E), (F), or (G) further includes a HFR3 sequence of SEQ ID NO:77, and optionally further includes a HFR1 sequence of SEQ ID NO:66, a HFR2 sequence of SEQ ID NO:67, and/or a HFR4 sequence of SEQ ID NO:71. In one aspect, the LCVR sequence of the antibody or antigen-binding fragment thereof of (A), (B), (C), (D), (E), (F), or (G) optionally further includes a LFR1 sequence of SEQ ID NO:72, a LFR2 sequence of SEQ ID NO:73, a LFR3 sequence of SEQ ID NO:74, and/or a LFR4 sequence of SEQ ID NO:75.

In another aspect, the HCVR sequence is SEQ ID NO:39 and/or the LCVR sequence is SEQ ID NO:41; or the HCVR sequence is SEQ ID NO:43 and/or the LCVR sequence is SEQ ID NO:45; or the HCVR sequence is SEQ ID NO:47 and/or, the LCVR sequence is SEQ ID NO: 49; or the HCVR sequence is SEQ ID NO:51 and/or the LCVR sequence is SEQ ID NO:53; or the HCVR sequence is SEQ ID NO:55 and/or the LCVR sequence is SEQ ID NO:57; or the HCVR sequence is SEQ ID NO:59 and/or the LCVR sequence is SEQ ID NO:61; or the HCVR sequence is SEQ ID NO:63 and/or the LCVR sequence is SEQ ID NO:65. In some aspects, the antibody includes: the HCVR sequence of SEQ ID NO:39 and the LCVR sequence of SEQ ID NO:41; or the HCVR sequence of SEQ ID NO:43 and the LCVR sequence of SEQ ID NO:45; or the HCVR sequence of SEQ ID NO:47 and the LCVR sequence of SEQ ID NO:49; or the HCVR sequence of SEQ ID NO:51 and the LCVR sequence of SEQ ID NO:53; or the HCVR sequence of SEQ ID NO:55 and the LCVR sequence of SEQ ID NO:57; or the HCVR sequence of SEQ ID NO:59 and the LCVR sequence of SEQ ID NO:61; or the HCVR sequence of SEQ ID NO:63 and the LCVR sequence of SEQ ID NO:65.

In one particular embodiment, the present invention provides an isolated monoclonal antibody or an antigen-binding fragment thereof comprising a HCVR, including a HCVR CDR1 sequence of SEQ ID NO:3, a HCVR CDR2 sequence of SEQ ID NO:10, and a HCVR CDR3 sequence of SEQ ID NO:12; and a LCVR, including a LCVR CDR1 sequence of SEQ ID NO:15, a LCVR CDR2 sequence of SEQ ID NO:23, and a LCVR CDR3 sequence of SEQ ID NO:32, wherein the antibody or antigen-binding fragment thereof specifically binds Gal9. In one aspect, the HCVR sequence of the antibody or antigen-binding fragment thereof further includes a HFR3 sequence of SEQ ID NO:68, and optionally further includes a HFR1 sequence of SEQ ID NO:66, a HFR2 sequence of SEQ ID NO:67, and/or a HFR4 sequence of SEQ ID NO:71. In one embodiment, the HCVR sequence is SEQ ID NO:39. In other aspects, the LCVR sequence of the antibody or antigen-binding fragment thereof optionally further includes a LFR1 sequence of SEQ ID NO:72, a LFR2 sequence of SEQ ID NO:73, a LFR3 sequence of SEQ ID NO:74, and/or a LFR4 sequence of SEQ ID NO:75. In one embodiment, the LCVR sequence is SEQ ID NO:41. The antibody is particularly exemplified herein as having the heavy chain sequence of SEQ ID NO:38 and the light chain sequence of SEQ ID NO:40 (Ab 001).

In another particular embodiment, the present invention provides an isolated monoclonal antibody or an antigen-binding fragment thereof comprising a HCVR, comprising a HCVR CDR1 sequence of SEQ ID NO:5, a HCVR CDR2 sequence of SEQ ID NO:9, and a HCVR CDR3 sequence of SEQ ID NO:12; and, a light chain variable region (LCVR), comprising a LCVR CDR1 sequence of SEQ ID NO:16, a LCVR CDR2 sequence of SEQ ID NO:25, and a LCVR CDR3 sequence of SEQ ID NO:33, wherein the antibody or antigen-binding fragment thereof specifically binds Gal9. In one aspect, the HCVR sequence of the antibody or antigen-binding fragment thereof further includes a HFR3 sequence of SEQ ID NO:69, and optionally further includes a HFR1 sequence of SEQ ID NO:66, a HFR2 sequence of SEQ ID NO:67, and/or a HFR4 sequence of SEQ ID NO:71. In one embodiment, the HCVR sequence is SEQ ID NO:47. In other aspects, the LCVR sequence of the antibody or antigen-binding fragment thereof optionally further includes a LFR1 sequence of SEQ ID NO:72, a LFR2 sequence of SEQ ID NO:73, a LFR3 sequence of SEQ ID NO:74, and/or a LFR4 sequence of SEQ ID NO:75. In one embodiment, the LCVR sequence is SEQ ID NO:49. The antibody is particularly exemplified herein as having the heavy chain sequence of SEQ ID NO:46 and the light chain sequence of SEQ ID NO:48 (Ab 003).

In one aspect, the antibody is a humanized antibody.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies are human immunoglobulins (recipient antibody) in which residues from a complementarity determining region (CDR) of the recipient are replaced by residues from a CDR of a non-human species (donor antibody) such as mouse, rat, or rabbit having the desired specificity, affinity, and capacity. In some instances, framework region (FR) residues of the human immunoglobulin are replaced by corresponding non-human residues. Furthermore, humanized antibodies may comprise residues which are found neither in the recipient antibody nor in the imported CDR or framework sequences. These modifications are made to further refine and maximize antibody manufacturability, stability, and/or performance. In general, the humanized antibody will comprise substantially all of at least one, and typically two, variable domains, in which all or substantially all of the CDRs correspond to those of a non-human immunoglobulin and all or substantially all of the FRs are those of a human immunoglobulin sequence. The humanized antibody optimally also will comprise at least a portion of an immunoglobulin constant region (Fc), typically that of a human immunoglobulin. For further details, see Jones et al., Nature, 321:522-525 (1986); Reichmann et al., Nature, 332:323-329 (1988); and Presta, Curr. Op. Struct. Biol., 2:593-596 (1992). The humanized antibody includes a PRIMATIZED™ antibody wherein the antigen-binding region of the antibody is derived from an antibody produced by immunizing macaque monkeys with the antigen of interest (see Weber, J et al., From rabbit antibody repertoires to rabbit monoclonal antibodies. Exp Mol Med 49, e305 (2017)).

Methods for humanizing non-human antibodies are well known in the art (see Yaghoub Safdari et al., (2013) Antibody humanization methods—a review and update, Biotechnology and Genetic Engineering Reviews, 29:2, 175-186, DOI: 10.1080/02648725.2013.801235). Preferably, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 [1988]), by substituting rodent CDRs or CDR sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework region (FR) for the humanized antibody (Sims et al., J. Immunol., 151:2296 (1993); Chothia et al., J. Mol. Biol., 196:901 [1987]). Another method uses a particular framework region derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 [1993]).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the CDR residues are directly and most substantially involved in influencing antigen binding.

Alternatively, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immuno., 7:33 (1993). Human antibodies can also be derived from antibody-display libraries, e.g., phage-display libraries (Hoogenboom et al., J. Mol. Biol., 227:381 (1991); Marks et al., J. Mol. Biol., 222:581-597 [1991], and Valldorf, Bernhard et al., “Antibody display technologies: selecting the cream of the crop” Biological Chemistry, vol. 403, no. 5-6, 2022, pp. 455-477).

Antibodies may be humanized by replacing sequences of the Fv variable region which are not directly involved in antigen binding with equivalent sequences from human Fv variable regions. General reviews of humanized chimeric antibodies are provided by Morrison et al., (Science 229:1202-1207 (1985)) and by Oi et al. (BioTechniques 4:214 (1986)). Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from for example, an antibody producing hybridoma. The recombinant DNA encoding the humanized or chimeric antibody, or fragment thereof, can then be cloned into an appropriate expression vector.

Humanized antibodies can alternatively be produced by CDR substitution (U.S. Pat. No. 5,225,539; Jones, Nature 321:552-525 (1986); Verhoeyan et al., Science 239:1534 (1988); and Beidler, J. Immunol. 141:4053-4060 (1988)).

In another aspect, the antigen-binding fragment thereof is a Fab, Fab′, F(ab′)₂, F_{d}, single chain Fv or scFv, disulfide linked Fᵥ, V-NAR domain, IgNar, intrabody, IgGACH₂, minibody, F(ab′)₃, tetrabody, triabody, diabody, single-domain antibody, DVD-Ig, Fcab, mAb₂, (scFv)₂, tandem scFv, DART®, TandAb, nanobody (see mdpi.com/2073-4468/8/2/28/pdf) or scFv-Fc.

Experimentally, antibodies can be cleaved with the proteolytic enzyme papain, which causes each of the heavy chains to break, producing three separate antibody fragments. “Antibody fragments” include a portion of an intact antibody, preferably the antigen binding or variable region of the intact antibody. Examples of antibody fragments include Fab, Fab′ and F(ab′)₂, Fc fragments or Fc-fusion products, single-chain Fvs (scFv), disulfide-linked Fvs (sdfv) and fragments including either a VL or VH domain; diabodies, tribodies and the like (Zapata et al. Protein Eng. 8(10):1057-1062 [1995]). The term “antibody fragment” or “antigen binding portion” (of antibody) includes, but is not limited to, fragments that are capable of binding antigen. The two units that consist of a light chain and a fragment of the heavy chain approximately equal in mass to the light chain are called the Fab fragments (i.e., the “antigen binding” fragments). The third unit, consisting of two equal segments of the heavy chain, is called the Fc fragment. The Fc fragment is typically not involved in antigen-antibody binding but is important in later processes involved in ridding the body of the antigen.

The Fab fragment contains the constant domain of the light chain and the first constant domain (CH1) of the heavy chain. “Fab” refers to an antibody fragment with a molecular mass of approximately 50,000 daltons and has an activity of binding to the antigen. It comprises approximately half of the N-terminal side of the heavy chain and the whole of the light chain connected by a disulfide bridge. The Fab can be obtained in particular by treatment of immunoglobulin by a protease, papain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group.

F(ab′)₂ antibody fragments originally were produced as pairs of Fab′ fragments which have hinge cysteines between them. Other chemical couplings of antibody fragments are also known. The term “F(ab′)₂” designates a fragment of approximately 100,000 daltons and an activity of binding to the antigen. This fragment is slightly larger than two Fab fragments connected via a disulfide bridge in the hinge region. These fragments are obtained by treating an immunoglobulin with a protease, pepsin. The Fab fragment can be obtained from the F(ab′)₂ fragment by cleaving of the disulfide bridge of the hinge region.

The Fc region of an antibody is the tail region of an antibody that interacts with cell surface receptors and some proteins of the complement system. This property allows antibodies to activate the immune system. In IgG, IgA and IgD antibody isotypes, the Fc region is composed of two identical protein fragments, derived from the second and third constant domains of the antibody's two heavy chains; IgM and IgE Fc regions contain three heavy chain constant domains (CH domains 2-4) in each polypeptide chain. The Fc regions of IgGs bear a highly conserved N-glycosylation site. Glycosylation of the Fc fragment is essential for Fc receptor-mediated activity. The N-glycans attached to this site are predominantly core-fucosylated diantennary structures of the complex type. In addition, small amounts of these N-glycans also bear bisecting GlcNAc and α-2,6 linked sialic acid residues.

Fc-Fusion proteins (also known as Fc chimeric fusion protein, Fc-Ig, Ig-based Chimeric Fusion protein and Fc-tag protein) are composed of the Fc domain of IgG genetically linked to a peptide or protein of interest. Fc-Fusion proteins have become valuable reagents for in vivo and in vitro research. The Fc-fused binding partner can range from a single peptide, a ligand that activates upon binding with a cell surface receptor, signaling molecules, the extracellular domain of a receptor that is activated upon dimerization or as a bait protein that is used to identify binding partners in a protein microarray. One of the most valuable features of the Fc domain in vivo, is it can dramatically prolong the plasma half-life of the protein of interest, which for bio-therapeutic drugs, results in an improved therapeutic efficacy; an attribute that has made Fc-Fusion proteins attractive bio-therapeutic agents.

The Fc fusion protein may be part of a pharmaceutical composition including an Fc fusion protein and a pharmaceutically acceptable carrier excipients or carrier. Pharmaceutically acceptable carriers, excipients or stabilizers are well known in the art (Remington's Pharmaceutical Sciences, 16th edition, Osol, A. Ed. (1980)). Acceptable carriers, excipients, or stabilizers are nontoxic to recipients at the dosages and concentrations employed, and may include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides, such as XTEN (see Haeckel A, et al., XTEN as Biological Alternative to PEGylation Allows Complete Expression of a Protease-Activatable Killin-Based Cytostatic. PLoS One. 2016 Jun. 13; 11(6):e 0157193. doi: 10.1371/journal.pone.0157193. PMID: 27295081; PMCID: PMC4905650); proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (for example, Zn-protein complexes); and/or non-ionic surfactants such as TWEEN™, PLURONICS™ or polyethylene glycol (PEG).

“Fv” is the minimum antibody fragment which contains a complete antigen-recognition and -binding site. The dimers of “scFv” correspond to two scFv molecules connected together by a peptide bond. This Fv chain is frequently the result of the expression of a fusion gene including the genes coding for VH and VL connected by a linker sequence coding a peptide. The human scFv fragment may include CDR regions that are maintained in an appropriate conformation, preferably by means of the use of genetic recombination techniques. This region consists of a dimer of one heavy- and one light-chain variable domain in tight, non-covalent association. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) may have the ability to recognize and bind antigen, although at a lower affinity than the entire binding site.

“Single-chain Fv” or “scFv” antibody fragments comprise the VH and VL domains of antibody, wherein these domains are present in a single polypeptide chain. Preferably, the Fv polypeptide further comprises a polypeptide linker between the VH and VL domains which enables the sFv to form the desired structure for antigen binding. For a review of sFv see Pluckthun in The Pharmacology of Monoclonal Antibodies, vol. 113, Rosenburg and Moore eds., Springer-Verlag, New York, pp. 269-315 (1994).

The “dsFv” fragment is a VH-VL heterodimer stabilized by a disulfide bridge; it may be divalent (dsFv₂). Fragments of divalent sc(Fv)₂ or multivalent antibodies may form spontaneously by the association of monovalent scFvs or be produced by connecting scFvs fragments by peptide binding sequences.

The Fc fragment is the support for the biological properties of the antibody, in particular its ability to be recognized by immunity effectors or to activate complement. It consists of constant fragments of the heavy chains beyond the hinge region.

The term “diabodies” signifies small antibody fragments having two antigen fixing sites. These fragments comprise, in the same VH-VL polypeptide chain, a variable heavy chain domain VH connected to a variable light chain domain VL. Using a binding sequence that is too short to allow the matching of two domains of the same chain, the matching with two complementary domains of another chain necessarily occurs and thus two antigen fixing sites are created.

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992) and Brennan et al., Science, 229:81 [1985]). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10:163-167 [1992]). According to another approach, F(ab′)₂ fragments can be isolated directly from recombinant host cell culture. Other techniques for the production of antibody fragments will be apparent to the skilled practitioner. In other embodiments, the antibody of choice is a single chain Fv fragment (scFv). See WO 93/16185.

In other aspects, the monoclonal antibody or antigen-binding fragment thereof binds human Gal9 with a K d of less than about 5 nM, 2 nM, 1 nM, 0.5 nM, 0.4 nM, 0.3 nM, 0.2 nM, 0.1 nM, 0.05 nM or 0.01 nM.

In some aspects, the affinity matured monoclonal antibody or antigen-binding fragment thereof has an affinity to Gal9 that is about 2-10-fold increased as compared to the non-affinity matured Ab A. For example, the affinity matured monoclonal antibody or antigen-binding fragment thereof has an affinity to Gal9 that is about 2-fold, 3-fold, 4-fold, 5-fold, 6-fold, 7-fold, 8-fold, 9-fold, 10-fold or more increased as compared to the non-affinity matured Ab A.

The term “antigen-binding domain” refers to the part of an antibody molecule that comprises the area specifically binding to or complementary to a part or all of an antigen. Where an antigen is large, an antibody may only bind to a particular part of the antigen. The “epitope” or “antigenic determinant” is a portion of an antigen molecule that is responsible for interactions with the antigen-binding domain of an antibody. An antigen-binding domain may be provided by one or more antibody variable domains (e.g., a so-called Fd antibody fragment consisting of a VH domain). An antigen-binding domain may comprise an antibody light chain variable region (VL) and an antibody heavy chain variable region (VH).

An “antigen” according to the invention covers any substance that will elicit an immune response. In particular, an “antigen” relates to any substance, preferably a peptide or protein, that reacts specifically with antibodies or T-lymphocytes (T cells). According to the present invention, the term “antigen” comprises any molecule which comprises at least one epitope. Preferably, an antigen in the context of the present invention is a molecule which, optionally after processing, induces an immune reaction. According to the present invention, any suitable antigen may be used, which is a candidate for an immune reaction, wherein the immune reaction is preferably a cellular immune reaction. In the context of the embodiments of the present invention, the antigen is preferably presented by a cell, preferably by an antigen presenting cell which includes a diseased cell, in particular a cancer cell, in the context of MHC molecules, which results in an immune reaction against the antigen. An antigen is preferably a product which corresponds to or is derived from a naturally occurring antigen. Such naturally occurring antigens include tumor antigens.

The term “epitope” refers to an antigenic determinant in a molecule such as an antigen, i.e., to a part in or fragment of the molecule that is recognized by the immune system. An epitope of a protein such as a tumor antigen preferably comprises a continuous or discontinuous portion of said protein.

The terms “epitope”, “antigen peptide”, “antigen epitope”, “immunogenic peptide” and “MHC binding peptide” can be used interchangeably herein and preferably relate to an incomplete representation of an antigen which is preferably capable of eliciting an immune response against the antigen or a cell expressing or comprising and preferably presenting the antigen.

The term “binding-affinity” generally refers to the strength of the sum total of noncovalent interactions between a single binding site of a molecule (e.g., an antibody), and its binding partner. A variety of methods of measuring binding affinity or binding activity are known in the art, any of which can be used for purposes of the present invention. Specific illustrative embodiments are described in the following.

As used herein, “specific binding” refers to antibody binding to a predetermined antigen. Typically, the antibody binds with an affinity corresponding to a K_{D} of about 10⁻⁸ M or less and binds to the predetermined antigen with an affinity (as expressed by K_{D}-dissociation constant) that is at least 10-fold less, and preferably at least 100-fold less than its affinity for binding to a non-specific antigen (e.g., BSA, casein) other than the predetermined antigen or a closely-related antigen. Alternatively, the antibody can bind with an affinity corresponding to a K_{A} of about 10⁶ M⁻¹, or about 10⁷ M⁻¹, or about 10⁸ M⁻¹, or 10⁹ M⁻¹ or higher, and binds to the predetermined antigen with an affinity (as expressed by K_{A}—association constant) that is at least 10 fold higher, and preferably at least 100 fold higher than its affinity for binding to a non-specific antigen (e.g., BSA, casein) other than the predetermined antigen or a closely-related antigen.

The term “k_{d}” (sec⁻¹), as used herein, is intended to refer to the dissociation rate constant of a particular antibody-antigen interaction. This value is also referred to as the off value. The term “K_{D}” (M⁻¹), as used herein, is intended to refer to the dissociation equilibrium constant of a particular antibody-antigen interaction.

In some embodiments, Gal9 antibody of the invention has a dissociation constant (K_{d}) of ≤5 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M) for Gal9, e.g., for human Gal9. In certain embodiments, Gal9 antibody has a dissociation constant (K_{d}) of ≤5 nM, ≤1 nM, ≤0.1 nM, ≤0.01 nM, or K 0.001 nM (e.g., 10⁻⁸ M or less, e.g., from 10⁻⁸ M to 10⁻¹³ M, e.g., from 10⁻⁹ M to 10⁻¹³ M) for Gal9, e.g., for human Gal9.

The term “kₐ” (M⁻¹sec⁻¹), as used herein, is intended to refer to the association rate constant of a particular antibody-antigen interaction. The term “K_{A}” (M), as used herein, is intended to refer to the association equilibrium constant of a particular antibody-antigen interaction.

The antibodies described herein may be characterized using surface plasmon resonance (SPR) assay or using solution equilibrium assay (SEA).

Surface plasmon resonance (SPR) is a physical phenomenon that occurs at the thin gold surface on an SPR chip. In an SPR assay, the binding target of the analyte—for example an antibody—is covalently attached to the surface with dextran linker. When the analyte is flowed past the surface, the binding that occurs between it and the surface can be measured directly as a change in local refractive index. This generates binding and dissociation curves as well as binding maximum data. SPR assays are typically more sensitive than plate-based assays and detect a much wider range of antibodies. This is primarily due to the lack of wash-steps that would typically remove most low-affinity target populations. The label-free nature of SPR also removes confounding effects from crosslinking labels, simplifying analysis and cutting down on false-negatives significantly. SPR assays also enable inline isotyping through the addition of anti-Isotype antibodies after the association phase.

SPR assay or label-free interaction measurements can be applied for the determination of antibodies specificity, studying thermodynamics of interaction, investigating mechanism of action, or for epitope characterization or affinity screening.

The ability to reliably estimate affinities with unpurified samples is a prerequisite for efficient screening. The use of high-throughput affinity-screening processes enables the reduction of overall project costs and duration by avoiding time-consuming purification steps and enabling the initial characterization of thousands of antibody candidates within a short time. Essential features of such an affinity-screening tool are (1) a reliable identification of affinity-improved candidates; (2) a reasonable equilibrium dissociation constant (KD) estimation of the respective clones; (3) compatibility with crude production matrices, such as bacterial extracts or cell culture supernatants; and (4) high-throughput processing (e.g., by automation of all sample-handling steps). Although state-of-the-art technologies, like surface plasmon resonance (SPR; e.g., Biacore) and bio-layer interferometry (BLI; e.g., Octet), partially fulfill these criteria,4,5 discrimination of affinities in the picomolar (pM) range in reasonable throughput remains a challenge.

Solution equilibrium titration (SET) or solution equilibrium assay (SEA) uses highly sensitive electrochemiluminescence as a readout system. Because the binding partners are not labeled, the resulting KD represents a sound approximation of the real affinity. For screening, diluted bacterial lysates or cell culture supernatants are equilibrated with four different concentrations of a soluble target molecule, and unbound antibodies are subsequently quantified on 384-well Meso Scale Discovery (MSD) plates coated with the respective antigen. For determination of KD values from the resulting titration curves, fit models deduced from the law of mass action for 1:1 and 2:1 binding modes are applied to assess hundreds of interactions simultaneously.

In one aspect, the antibody binds to Gal9 and inhibits Gal9 binding to a Gal9 receptor (e.g., TIM3 or CD44). In another aspect, the antibody binds to Gal9 and does not inhibit Gal9 binding to a Gal9 receptor (e.g., TIM3 or CD44). In some embodiments, a Gal9 antibody having any of the characteristics provided herein inhibits at least 25%, 50%, 75%, 80%, 90% or 100% of the signaling of Gal9.

In another aspect, the antibody reverses Gal9-induced Th1 apoptosis of T cells (such as CD4⁺ T cells).

In one aspect, the antibody suppresses Gal9-induced Treg expansion.

In another embodiment, the invention provides a method of treating cancer in a subject in need thereof including administering to the subject an effective amount of any one of the isolated monoclonal antibodies or antigen-binding fragments described herein, thereby treating cancer in the subject.

The term “subject” as used herein refers to any individual or patient to which the subject methods are performed. Generally, the subject is human, although as will be appreciated by those in the art, the subject may be an animal. Thus, other animals, including vertebrate such as rodents (including mice, rats, hamsters, and guinea pigs), cats, dogs, rabbits, farm animals including cows, horses, goats, sheep, pigs, chickens, etc., and primates (including monkeys, chimpanzees, orangutans, and gorillas) are included within the definition of subject.

The term “treatment” is used interchangeably herein with the term “therapeutic method” and refers to both 1) therapeutic treatments or measures that cure, slow down, lessen symptoms of, and/or halt progression of a diagnosed pathologic condition or disorder, and 2) prophylactic/preventative measures. Those in need of treatment may include individuals already having a particular medical disorder as well as those who may ultimately acquire the disorder (i.e., those needing preventive measures). “Treatment” covers any administration or application of a therapeutic for a disease (also referred to herein as a “disorder” or a “condition”) in a mammal, including a human, and includes inhibiting the disease or progression of the disease, inhibiting or slowing the disease or its progression, arresting its development, partially or fully relieving the disease, partially or fully relieving one or more symptoms of a disease, or restoring or repairing a lost, missing, or defective function; or stimulating an inefficient process. The term “treatment” also includes reducing the severity of any phenotypic characteristic and/or reducing the incidence, degree, or likelihood of that characteristic.

The terms “therapeutically effective amount”, “effective dose,” “therapeutically effective dose”, “effective amount,” or the like refer to that amount of the subject compound that will elicit the biological or medical response of a tissue, system, animal, or human that is being sought by the researcher, veterinarian, medical doctor, or other clinician. Generally, the response is either amelioration of symptoms in a patient or a desired biological outcome (e.g., treatment of the cancer). A therapeutically effective amount of Gal9 antibody and antigen binding fragment thereof of the invention may vary according to factors such as the disease state, age, sex, and weight of the individual, and the ability of the antibody and antigen binding fragment thereof to elicit a desired response in the individual. A therapeutically effective amount encompasses an amount in which any toxic or detrimental effects of Gal9 antibody and antigen binding fragment thereof are outweighed by the therapeutically beneficial effects.

The terms “administration of” and or “administering” should be understood to mean providing a pharmaceutical composition in a therapeutically effective amount to the subject in need of treatment. Administration routes can be enteral, topical or parenteral. As such, administration routes include but are not limited to intracutaneous, subcutaneous, intravenous, intramuscular, intravitreal, intraperitoneal, intraarterial, intrathecal, intracapsular, intraorbital, intracardiac, intradermal, transdermal, transtracheal, subcuticular, intraarticulare, subcapsular, subarachnoid, intraspinal and intrasternal, oral, sublingual buccal, rectal, vaginal, nasal ocular administrations, as well infusion, inhalation, and nebulization. The phrases “parenteral administration” and “administered parenterally” as used herein means modes of administration other than enteral and topical administration. In various embodiments, Gal9 antibodies may be administered subcutaneously or intravenously.

The antibody and antigen-binding fragments thereof described herein can be prepared in pharmaceutical compositions comprising the antibody or antigen-binding fragment thereof and a pharmaceutically acceptable carrier. A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed. For example, if the therapeutic agent is to be administered orally, the carrier may be a gel capsule. If the therapeutic agent is to be administered subcutaneously, the carrier ideally is not irritable to the skin and does not cause injection site reaction.

The pharmaceutical compositions can be administered in a variety of unit dosage forms depending upon the method of administration. Suitable unit dosage forms, include, but are not limited to powders, tablets, pills, capsules, lozenges, suppositories, patches, nasal sprays, injectables, implantable sustained-release formulations, lipid complexes, etc.

The subject compositions may be formulated into preparations in solid, semi-solid, liquid, or gaseous forms; including, but not limited to, tablets, capsules, powders, granules, ointments, solutions, suppositories, enemas, injections, inhalants, and aerosols.

In various embodiments, compositions comprising Gal9 antibody and antigen binding fragment thereof are provided in formulations with a wide variety of pharmaceutically acceptable carriers (see, e.g., Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7th ed., Lippencott Williams and Wilkins (2004); Kibbe et al., Handbook of Pharmaceutical Excipients, 3rd ed., Pharmaceutical Press (2000) Strickley RG, Lambert WJ. A review of Formulations of Commercially Available Antibodies. J Pharm Sci. 2021 July; 110(7):2590-2608.e56. doi: 10.1016/j.xphs.2021.03.017. Epub 2021 Mar. 28. PMID: 33789155). Various pharmaceutically acceptable carriers, which include vehicles, adjuvants, and diluents, are available. Moreover, various pharmaceutically acceptable auxiliary substances, such as pH adjusting and buffering agents, tonicity adjusting agents, stabilizers, wetting agents and the like, are also available. Non-limiting exemplary carriers include saline, buffered saline, dextrose, water, glycerol, ethanol, and combinations thereof.

In various embodiments, compositions comprising Gal9 antibody and antigen binding fragment thereof may be formulated for injection, including subcutaneous administration, by dissolving, suspending, or emulsifying them in an aqueous or nonaqueous solvent, such as vegetable or other oils, synthetic aliphatic acid glycerides, esters of higher aliphatic acids, or propylene glycol; and if desired, with conventional additives such as solubilizers, isotonic agents, suspending agents, emulsifying agents, stabilizers and preservatives.

In various embodiments, the compositions may be formulated for inhalation, for example, using pressurized acceptable propellants such as dichlorodifiuoromethane, propane, nitrogen, and the like.

The compositions may also be formulated, in various embodiments, into sustained release microcapsules, such as with biodegradable or non-biodegradable polymers. A non-limiting exemplary biodegradable formulation includes poly lactic acid-glycolic acid (PLGA) polymer. A non-limiting exemplary non-biodegradable formulation includes a polyglycerin fatty acid ester. Certain methods of making such formulations are described, for example, in EP 1125584 A1.

Pharmaceutical dosage packs comprising one or more containers, each containing one or more doses of Gal9 antibody and antigen binding fragments thereof, are also provided. In some embodiments, a unit dosage is provided wherein the unit dosage contains a predetermined amount of a composition comprising Gal9 antibody and antigen binding fragments thereof, with or without one or more additional agents. In some embodiments, such a unit dosage is supplied in single-use prefilled syringe for injection. In various embodiments, the composition contained in the unit dosage may comprise saline, sucrose, or the like; a buffer, such as phosphate, or the like; and/or be formulated within a stable and effective pH range. Alternatively, in some embodiments, the composition may be provided as a lyophilized powder that may be reconstituted upon addition of an appropriate liquid, for example, sterile water. In some embodiments, the composition comprises one or more substances that inhibit protein aggregation, including, but not limited to, sucrose and arginine. In some embodiments, a composition of the invention comprises heparin and/or a proteoglycan.

Pharmaceutical compositions are administered in an amount effective for treatment or prophylaxis of the specific indication. The therapeutically effective amount is typically dependent on the weight of the subject being treated, his or her physical or health condition, the extensiveness of the condition to be treated, or the age of the subject being treated.

In some embodiments, Gal9 antibody and antigen binding fragments thereof may be administered in an amount in the range of about 50 g/kg body weight to about 50 mg/kg body weight per dose. In some embodiments, Gal9 antibody and antigen binding fragments thereof may be administered in an amount in the range of about 100 g/kg body weight to about 50 mg/kg body weight per dose. In some embodiments, Gal9 antibody and antigen binding fragments thereof may be administered in an amount in the range of about 100 g/kg body weight to about 20 mg/kg body weight per dose. In some embodiments, Gal9 antibody and antigen binding fragments thereof may be administered in an amount in the range of about 0.5 mg/kg body weight to about 20 mg/kg body weight per dose.

In some embodiments, Gal9 antibody and antigen binding fragments thereof may be administered in an amount in the range of about 10 mg to about 1,000 mg per dose. In some embodiments, Gal9 antibody and antigen binding fragments thereof may be administered in an amount in the range of about 20 mg to about 500 mg per dose. In some embodiments, Gal9 antibody and antigen binding fragments thereof may be administered in an amount in the range of about 20 mg to about 300 mg per dose. In some embodiments, Gal9 antibody and antigen binding fragments thereof may be administered in an amount in the range of about 20 mg to about 200 mg per dose.

The Gal9 antibody and antigen binding fragments thereof compositions may be administered as needed to subjects. In some embodiments, an effective dose of Gal9 antibody and antigen binding fragments thereof is administered to a subject one or more times. In various embodiments, an effective dose of Gal9 antibody and antigen binding fragments thereof is administered to the subject once a month, less than once a month, such as, for example, every two months, every three months, or every six months. In other embodiments, an effective dose of Gal9 antibody and antigen binding fragments thereof is administered more than once a month, such as, for example, every two weeks, every week, twice per week, three times per week, daily, or multiple times per day. An effective dose of Gal9 antibody and antigen binding fragments thereof is administered to the subject at least once. In some embodiments, the effective dose of Gal9 antibody and antigen binding fragment thereof may be administered multiple times, including for periods of at least a month, at least six months, or at least a year. In some embodiments, Gal9 antibody and antigen binding fragments thereof is administered to a subject as needed to alleviate one or more symptoms of a condition.

The antibodies described herein can be used for the treatment of cancer.

Cancer is a group of diseases involving abnormal cell growth with the potential to invade or spread to other parts of the body. In 2015, about 90.5 million people had cancer, about 14.1 million new cases occur a year and it caused about 8.8 million deaths (15.7% of deaths). The most common types of cancer in males are lung cancer, prostate cancer, colorectal cancer and stomach cancer. In females, the most common types are breast cancer, colorectal cancer, lung cancer and cervical cancer.

The term “cancer” refers to a group of diseases characterized by abnormal and uncontrolled cell proliferation starting at one site (primary site) with the potential to invade and to spread to other sites (secondary sites, metastases) which differentiate cancer (malignant tumor) from benign tumor. Virtually all the organs can be affected, leading to more than 100 types of cancer that can affect humans. Cancers can result from many causes including genetic predisposition, viral infection, exposure to ionizing radiation, exposure to environmental pollutant, tobacco and or alcohol use, obesity, poor diet, lack of physical activity or any combination thereof.

As used herein, “neoplasm” or “tumor” including grammatical variations thereof, means new and abnormal growth of tissue, which may be benign or cancerous. In a related aspect, the neoplasm is indicative of a neoplastic disease or disorder, including but not limited to various cancers. For example, such cancers can include prostate, pancreatic, biliary, colon, rectal, liver, kidney, lung, testicular, breast, ovarian, pancreatic, brain, and head and neck cancers, melanoma, sarcoma, multiple myeloma, leukemia, lymphoma, and the like.

In one aspect, the cancer is a hematological cancer or a solid tumor.

Cancer that begins in blood-forming tissue, such as the bone marrow, or in the cells of the immune system are referred to as hematological cancer, or blood cancer. Hematological cancers affect the production and function of blood cells, and are classified in three main types: leukemia, lymphoma, and multiple myeloma.

As used herein, “leukemia” refers to a blood cancer caused by the rapid production of abnormal white blood cells. Examples of leukemia include acute lymphoblastic leukemia (ALL), acute myeloid leukemia (AML), chronic lymphocytic leukemia, chronic myelogenous leukemia, and hairy cell leukemia.

As used herein, “lymphoma” refers to a type of blood cancer that affects the lymphatic system. Examples of lymphoma include AIDS-related lymphoma, cutaneous T-cell lymphoma, Hodgkin lymphoma, Hodgkin lymphoma, mycosis fungoides, non-Hodgkin lymphoma, primary central nervous system lymphoma, Sézary syndrome, cutaneous T-Cell lymphoma, and Waldenström macroglobulinemia.

As used herein, “myeloma” is a cancer of the plasma cells. Examples of myeloma include chronic myeloproliferative neoplasms, Langerhans cell histiocytosis, multiple myeloma, plasma cell neoplasm, myelodysplastic syndromes, and myelodysplastic/myeloproliferative neoplasms.

In some embodiments, the Gal9 antibody and antigen binding fragment thereof of the invention can be used alone, or alternatively used in combination with any other suitable compound known to be able to treat the disease or indication.

In some aspects administration can be in combination with one or more additional therapeutic agents. The phrases “combination therapy”, “combined with” and the like refer to the use of more than one medication or treatment simultaneously to increase the response. The composition of the present invention might for example be used in combination with other drugs or treatment in use to treat cancer. Specifically, the administration of the composition of the present invention to a subject can be in combination with any anti-cancer therapies. Such therapies can be administered prior to, simultaneously with, or following administration of the composition of the present invention.

In certain embodiments, Gal9 antibody is administered with another treatment, either simultaneously, or consecutively, to a subject, e.g., a subject having cancer. For example, Gal9 antibody may be administered with one of more of: radiotherapy, surgery, or chemotherapy, e.g., targeted chemotherapy or immunotherapy. The administration of the two agents may start at times that are, e.g., 30 minutes, 60 minutes, 90 minutes, 120 minutes, 3 hours, 6 hours, 12 hours, 24 hours, 36 hours, 48 hours, 3 days, 5 days, 7 days, or one or more weeks apart, or administration of the second agent may start, e.g., 30 minutes, 60 minutes, 90 minutes, 120 minutes, 3 hours, 6 hours, 12 hours, 24 hours, 36 hours, 48 hours, 3 days, 5 days, 7 days, or one or more weeks after the first agent has been administered.

In some aspects, the hematological cancer is selected from the group consisting of acute lymphoblastic leukemia (ALL), acute myeloid leukemia (AML), chronic lymphocytic leukemia, chronic myelogenous leukemia, and hairy cell leukemia, AIDS-related lymphoma, cutaneous T-cell lymphoma, Hodgkin lymphoma, Hodgkin lymphoma, mycosis fungoides, non-Hodgkin lymphoma, primary central nervous system lymphoma, Sézary syndrome, cutaneous T-Cell lymphoma, Waldenström macroglobulinemia, diffuse large B-Cell Lymphoma (DLBCL) and multiple myeloma.

The French-American-British (FAB) classification of acute myeloid leukemia (AML) categorizes AML into different stages of the disease. FAB subtypes are shown in Table 1.

TABLE 1
FAB Classification

| FAB Subtype | Description |
|---|---|
| M0 | Undifferentiated acute myeloblastic leukemia |
| M1 | Acute myeloblastic leukemia with minimal maturation |
| M2 | Acute myeloblastic leukemia with maturation |
| M3 | Acute promyelocytic leukemia (APL) |
| M4 | Acute myelomoncytic leukemia |
| M5 | Acute monocytic leukemia |
| M6 | Acute erythroblastic leukemia |
| M7 | Acute megakaryoblastic leukemia |

Examples of solid cancer include but are not limited to, carcinoma, sarcoma, squamous cell cancer, small-cell lung cancer, pituitary cancer, esophageal cancer, astrocytoma, soft tissue sarcoma, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, renal cancer, liver cancer, prostate cancer, vulval cancer, thyroid cancer, hepatic carcinoma, brain cancer, endometrial cancer, testis cancer, cholangiocarcinoma, gallbladder carcinoma, gastric cancer, melanoma, gastroesophageal cancer, esophageal cancer, cervical cancer, and various types of head and neck cancer.

In other aspects, the solid tumor is selected from the group consisting of breast cancer, head and neck cancer, lung cancer, melanoma, uveal melanoma, colorectal cancer, renal carcinoma, urothelial cancer, ovarian cancer, endometrial cancer, liver cancer, pancreatic cancer, cholangiocarcinoma, gastric cancer, gastroesophageal cancer, esophageal cancer, cervical cancer, head and neck squamous cancer, and prostate cancer.

In one aspect, the method further includes administering to the patient a chemotherapeutic agent, an anti-angiogenesis agent, a growth inhibitory agent, an immune-oncology agent, a checkpoint inhibitor and/or an anti-neoplastic composition.

A “chemotherapeutic agent” is a chemical compound that can be useful in the treatment of cancer. Examples of chemotherapeutic agents include, but are not limited to, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammal1 and calicheamicin omegal1 (see, e.g., Agnew, Chem Intl. Ed. Engl, 33: 183-186 (1994); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; polysaccharide complex (JHS Natural Products, Eugene, OR); razoxane; rhizoxin; sizofiran; spirogermanium; tenuazonic acid; triaziquone; 2, 2′, 2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel (Bristol-Myers Squibb Oncology, Princeton, N.J.), Cremophor-free, albumin-engineered nanoparticle formulation of paclitaxel (American Pharmaceutical Partners, Schaumberg, Illinois), nab-paclitaxel (Celgene), and doxetaxel (Rhone-Poulenc Rorer, Antony, France); chloranbucil; gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluorometlhylornithine (DMFO); retinoids such as retinoic acid; capecitabine; combretastatin; leucovorin (LV); oxaliplatin, including the oxaliplatin treatment regimen (FOLFOX); inhibitors of PKC-alpha, Raf, H-Ras, EGFR (e.g., erlotinib) and VEGF-A that reduce cell proliferation and pharmaceutically acceptable salts, acids or derivatives of any of the above.

Further non-limiting exemplary chemotherapeutic agents include anti-hormonal agents that act to regulate or inhibit hormone action on cancers such as anti-estrogens and selective estrogen receptor modulators (SERMs), including, for example, tamoxifen (including tamoxifen), raloxifene, droloxifene, 4-hydroxy tamoxifen, trioxifene, keoxifene, LY117018, onapristone, and toremifene; aromatase inhibitors that inhibit the enzyme aromatase, which regulates estrogen production in the adrenal glands, such as, for example, 4 (5)-imidazoles, aminoglutethimide, megestrol acetate, exemestane, formestanie, fadrozole, vorozole, letrozole, and anastrozole; and anti-androgens such as flutamide, nilutamide, bicalutamide, leuprolide, and goserelin; as well as troxacitabine (a 1, 3-dioxolane nucleoside cytosine analog); antisense oligonucleotides, particularly those which inhibit expression of genes in signaling pathways implicated in aberrant cell proliferation, such as, for example, PKC-alpha, Ralf and H-Ras; ribozymes such as a VEGF expression inhibitor (e.g., ribozyme) and a HER2 expression inhibitor; vaccines such as gene therapy vaccines, for example, vaccine, vaccine, and vaccine; rIL-2; topoisomerase 1 inhibitor; rmRH; and pharmaceutically acceptable salts, acids or derivatives of any of the above.

An “anti-angiogenesis agent” or “angiogenesis inhibitor” refers to a small molecular weight substance, a polynucleotide (including, e.g., an inhibitory RNA (RNAi or siRNA), a polypeptide, an isolated protein, a recombinant protein, an antibody, or conjugates or fusion proteins thereof, that inhibits angiogenesis, vasculogenesis, or undesirable vascular permeability, either directly or indirectly. It should be understood that the anti-angiogenesis agent includes those agents that bind and block the angiogenic activity of the angiogenic factor or its receptor. For example, an anti-angiogenesis agent is an antibody or other antagonist to an angiogenic agent, e.g., antibodies to VEGF-A (e.g., bevacizumab) or to the VEGF-A receptor (e.g., KDR receptor or Flt-1 receptor), anti-PDGFR inhibitors such as (Imatinib Mesylate), small molecules that block VEGF receptor signaling (e.g., PTK787/ZK2284, SU6668, /SUI 1248 (sunitinib malate), AMG706, or those described in, e.g., international patent application WO 2004/113304). Anti-angiogenesis agents also include native angiogenesis inhibitors, e.g., angiostatin, endostatin, etc. See, e.g., Klagsbrun and D'Amore (1991) Annu. Rev. Physiol. 53: 217-39; Streit and Detmar (2003) Oncogene 22: 3172-3179 (e.g., Table 3 listing anti-angiogenic therapy in malignant melanoma); Ferrara & Alitalo (1999) Nature Medicine 5 (12): 1359-1364; Tonini et al. (2003) Oncogene 22: 6549-6556 (e.g., Table 2 listing known anti-angiogenic factors); and Sato (2003) Int. J. Clin. Oncol. 8: 200-206 (e.g., Table 1 listing anti-angiogenic agents used in clinical trials).

A “growth inhibitory agent” as used herein refers to a compound or composition that inhibits growth of a cell (such as a cell expressing VEGF) either in vitro or in vivo. Thus, the growth inhibitory agent may be one that significantly reduces the percentage of cells (such as a cell expressing VEGF) in S phase. Examples of growth inhibitory agents include, but are not limited to, agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in Mendelsohn and Israel, eds., The Molecular Basis of Cancer, Chapter 1, entitled “Cell cycle regulation, oncogenes, and antineoplastic drugs” by Murakami et al. (W.B. Saunders, Philadelphia, 1995), e.g., p. 13. The taxanes (paclitaxel and docetaxel) are anticancer drugs both derived from the yew tree. Docetaxel (Rhone-Poulenc Rorer), derived from the European yew, is a semisynthetic analogue of paclitaxel (Bristol-Myers Squibb). Paclitaxel and docetaxel promote the assembly of microtubules from tubulin dimers and stabilize microtubules by preventing depolymerization, which results in the inhibition of mitosis in cells.

The term “anti-neoplastic composition” refers to a composition useful in treating cancer comprising at least one active therapeutic agent. Examples of therapeutic agents include, but are not limited to, e.g., chemotherapeutic agents, growth inhibitory agents, cytotoxic agents, agents used in radiation therapy, anti-angiogenesis agents, cancer immunotherapeutic agents (also referred to as immuno-oncology agents), apoptotic agents, anti-tubulin agents, and other-agents to treat cancer, such as anti-HER-2 antibodies, anti-CD20 antibodies, an epidermal growth factor receptor (EGFR) antagonist (e.g., a tyrosine kinase inhibitor), HER1/EGFR inhibitor (e.g., erlotinib platelet derived growth factor inhibitors (e.g., Imatinib Mesylate), a COX-2 inhibitor (e.g., celecoxib), interferons, CTLA4 inhibitors (e.g., anti-CTLA antibody ipilimumab), PD-1 inhibitors (e.g., anti-PD-1 antibodies, BMS-936558), PD-L1 inhibitors (e.g., anti-PD-L1 antibodies, MPDL3280A), PD-L2 inhibitors (e.g., anti-PD-L2 antibodies), VISTA inhibitors (e.g., anti-VISTA antibodies), cytokines, antagonists (e.g., neutralizing antibodies) that bind to one or more of the following targets ErbB2, ErbB3, ErbB4, PDGFR-beta, BlyS, APRIL, BCMA, PD-1, PD-L1, PD-L2, CTLA4, VISTA, or VEGF receptor (s), TRAIL/Apo2, and other bioactive and organic chemical agents, etc. Combinations thereof are also included in the invention.

“Checkpoint inhibitor therapy” is a form of cancer treatment currently that uses immune checkpoints which affect immune system functioning. Immune checkpoints can be stimulatory or inhibitory. Tumors can use these checkpoints to protect themselves from immune system attacks. Checkpoint therapy can block inhibitory checkpoints, restoring immune system function. Checkpoint proteins include programmed cell death 1 protein (PDCD1, PD-1; also known as CD279) and its ligand, PD-1 ligand 1 (PD-L1, CD274), cytotoxic T-lymphocyte-associated protein 4 (CTLA-4), A2AR (Adenosine A2A receptor), B7-H3 (or CD276), B7-H4 (or VTCN1), BTLA (B and T Lymphocyte Attenuator, or CD272), IDO (Indoleamine 2,3-dioxygenase), KIR (Killer-cell Immunoglobulin-like Receptor), LAG3 (Lymphocyte Activation Gene-3), TIM-3 (T-cell Immunoglobulin domain and Mucin domain 3), and VISTA (V-domain Ig suppressor of T cell activation).

Programmed cell death protein 1, also known as PD-1 and CD279 (cluster of differentiation 279), is a cell surface receptor that plays an important role in down-regulating the immune system and promoting self-tolerance by suppressing T cell inflammatory activity. PD-1 is an immune checkpoint and guards against autoimmunity through a dual mechanism of promoting apoptosis (programmed cell death) in antigen-specific T-cells in lymph nodes while simultaneously reducing apoptosis in regulatory T cells (anti-inflammatory, suppressive T cells).

PD-1 has two ligands, PD-L1 and PD-L2, which are members of the B7 family. PD-L1 protein is upregulated on macrophages and dendritic cells (DC) in response to LPS and GM-CSF treatment, and on T cells and B cells upon TCR and B cell receptor signaling, whereas in resting mice, PD-L1 mRNA can be detected in the heart, lung, thymus, spleen, and kidney. PD-L1 is expressed on almost all murine tumor cell lines, including PA1 myeloma, P815 mastocytoma, and B16 melanoma upon treatment with IFN-7. PD-L2 expression is more restricted and is expressed mainly by DCs and a few tumor lines.

CTLA4 or CTLA-4 (cytotoxic T-lymphocyte-associated protein 4), also known as CD152 (cluster of differentiation 152), is a protein receptor that, functioning as an immune checkpoint, downregulates immune responses. CTLA4 is constitutively expressed in regulatory T cells but only upregulated in conventional T cells after activation—a phenomenon which is particularly notable in cancers. CTLA4 is a member of the immunoglobulin superfamily that is expressed by activated T cells and transmits an inhibitory signal to T cells. CTLA4 is homologous to the T-cell co-stimulatory protein, CD28, and both molecules bind to CD80 and CD86, also called B7-1 and B7-2 respectively, on antigen-presenting cells. CTLA-4 binds CD80 and CD86 with greater affinity and avidity than CD28 thus enabling it to outcompete CD28 for its ligands. CTLA4 transmits an inhibitory signal to T cells, whereas CD28 transmits a stimulatory signal. CTLA4 is also found in regulatory T cells and contributes to its inhibitory function. T cell activation through the T cell receptor and CD28 leads to increased expression of CTLA-4.

There are several checkpoint inhibitors that are currently used to treat cancer. PD-1 inhibitors include Pembrolizumab (Keytruda) and Nivolumab (Opdivo). PD-L1 inhibitors include Atezolizumab (Tecentriq), Avelumab (Bavencio) and Durvalumab (Imfinzi). CTLA-4 inhibitors include Iplimumab (Yervoy). There are several other checkpoint inhibitors being developed including an anti B7-H3 antibody (MGA271), an anti-KIR antibody (Lirilumab) and an anti-LAG3 antibody (BMS-986016).

As used herein, a “immune-oncology agent” refers to an anti-cancer agent that specifically targets cancer by targeting the immune system; this generally refers to therapeutic antibodies. In some aspect, the immune-oncology agent is an anti-glucocorticoid-induced tumor necrosis factor receptor family-related protein (GITR) antibody.

Tumor necrosis factor receptor superfamily member 18 (TNFRSF18), also known as glucocorticoid-induced TNFR-related protein (GITR) or CD357, also called activation-inducible TNFR family receptor (AITR), is encoded by the TNFRSF18 gene at chromosome 1.

GITR is a member of TNFR superfamily and shares high homology in cytoplasmic domain, characterized with cysteine pseudo-repeats, with other members of TNFRSF, such as CD137, OX40 or CD27. GITR is constitutively expressed on CD25+CD4+ regulatory T cells and its expression is upregulated on all T cell subsets after activation. GITR is also expressed on murine neutrophils and NK cells. GITR interacts with its ligand (GITRL) that is expressed on antigen-presenting cells (APC) and endothelial cells.

GITR is an immune checkpoint molecule that has potential in cancer treatment. GITR signaling can promote antitumor and anti-infective immune response, but also can be a driver of autoimmune diseases. Different responses to GITR signaling rely on the GITR expression on different immune cell types. How GITR signaling is modulated in the different cells remains unknown. GITR agonistic antibodies are in the clinical trials as activators of effector CD8 T cells, while decreasing number of circulating suppressive regulatory T cells. Limited response to GITR agonistic antibodies is enhanced in combination with anti-PD-1 or anti-CTLA-4 therapies.

In an additional embodiment, the invention provides a polynucleotide encoding the heavy chain or the light chain or the antigen-binding portion thereof of any one of the antibodies or antigen-binding fragments thereof described herein.

The invention also provides nucleic acid molecules comprising polynucleotides that encode one or more chains of an antibody described herein, such as an anti-Gal9 antibody. In some embodiments, a nucleic acid molecule comprises a polynucleotide that encodes a heavy chain or a light chain of an antibody described herein. In some embodiments, a nucleic acid molecule comprises both a polynucleotide that encodes a heavy chain and a polynucleotide that encodes a light chain, of an antibody described herein. In some embodiments, a first nucleic acid molecule comprises a first polynucleotide that encodes a heavy chain and a second nucleic acid molecule comprises a second polynucleotide that encodes a light chain.

In some such embodiments, the heavy chain and the light chain are expressed from one nucleic acid molecule, or from two separate nucleic acid molecules, as two separate polypeptides. In some embodiments, such as when an antibody is an scFv, a single polynucleotide encodes a single polypeptide comprising both a heavy chain and a light chain linked together.

In some embodiments, a polynucleotide encoding a heavy chain or light chain of an antibody described herein comprises a nucleotide sequence that encodes a leader sequence, which, when translated, is located at the N-terminus of the heavy chain or light chain. As discussed above, the leader sequence may be the native heavy or light chain leader sequence or may be another heterologous leader sequence.

As used herein, the term “nucleic acid” or“oligonucleotide” refers to polynucleotides such as deoxyribonucleic acid (DNA) or ribonucleic acid (RNA). Nucleic acids include but are not limited to genomic DNA, cDNA, mRNA, iRNA, miRNA, tRNA, ncRNA, rRNA, and recombinantly produced and chemically synthesized molecules such as aptamers, plasmids, anti-sense DNA strands, shRNA, ribozymes, nucleic acids conjugated and oligonucleotides. According to the invention, a nucleic acid may be present as a single-stranded or double-stranded and linear or covalently circularly closed molecule. A nucleic acid can be isolated. The term “isolated nucleic acid” means, that the nucleic acid (i) was amplified in vitro, for example via polymerase chain reaction (PCR), (ii) was produced recombinantly by cloning, (iii) was purified, for example, by cleavage and separation by gel electrophoresis, (iv) was synthesized, for example, by chemical synthesis, or (vi) extracted from a sample. A nucleic might be employed for introduction into, i.e., transfection of, cells, in particular, in the form of RNA which can be prepared by in vitro transcription from a DNA template. The RNA can moreover be modified before application by stabilizing sequences, capping, and polyadenylation.

The term “sample” may include whole blood, plasma, serum, buffy coat, a body fluid, lymphocytes, tissue, amniotic fluid, cultured cells, and the like. The term “sample” may also refer to a urine sample, saliva sample, blood cell-free DNA and specimen of or from the skin, mucous membrane or other body area or surface to be examined by means of a swab. In various embodiments, the means used to collect the sample may contain a preservative. The preservative may include preservatives such as hydrochloric acid, boric acid, acetic acid, toluene, or thymol.

The nucleic acid may be extracted from the sample, by any method known in the art including by using organic solvents such as a mixture of phenol and chloroform, followed by precipitation with ethanol. Among other methods of extracting cell-free nucleic acid, one such method includes, for example, using polylysine-coated silica particles. Alternatively, the cell-free DNA may be extracted using commercially available kit such as, for example, QIAamp® DNA minikit (Qiagen, Germantown, MD).

The extracted nucleic acid is amplified by one of the alternative methods for amplification well known in the art, which include for example: the Xmap® technology of Luminex that allows the simultaneous analysis of up to 500 bioassays through the reading of biological test on the surface of microscopic polystyrene bead; the multiplex PCR that allows the simultaneous amplification of several DNA sequences; the multiplex ligation-dependent probe amplification (MLPA) for the amplification of multiple targets using a single pair of primers; the quantitative PCR (qPCR), which measures and quantify the amplification in real time; the ligation chain reaction (LCR) that uses primers covering the entire sequence to amplify, thereby preventing the amplification of sequences with a mutation; the rolling circle amplification (RCA), wherein the two ends of the sequences are joined by a ligase prior to the amplification of the circular DNA; the helicase dependent amplification (HDA) which relies on a helicase for the separation of the double stranded DNA; the loop mediated isothermal amplification (LAMP) which employs a DNA polymerase with high strand displacement activity; the nucleic acid sequence based amplification, specifically designed for RNA targets; the strand displacement amplification (SDA) which relies on a strand-displacing DNA polymerase, to initiate replication at nicks created by a strand-limited restriction endonuclease or nicking enzyme at a site contained in a primer; and the multiple displacement amplification (MDA), based on the use of the highly processive and strand displacing DNA polymerase from the bacteriophage Ø29. amplification methods as used herein have been used and tested, and are well known in the art.

As used herein “amplified DNA” or “PCR product” refers to an amplified fragment of DNA of defined size. Various techniques are available and well known in the art to detect PCR products. PCR product detection methods include, but are not restricted to, gel electrophoresis using agarose or polyacrylamide gel and adding ethidium bromide staining (a DNA intercalant), labeled probes (radioactive or non-radioactive labels, southern blotting), labeled deoxyribonucleotides (for the direct incorporation of radioactive or non-radioactive labels) or silver staining for the direct visualization of the amplified PCR products; restriction endonuclease digestion, that relies agarose or polyacrylamide gel or High-performance liquid chromatography (HPLC); dot blots, using the hybridization of the amplified DNA on specific labeled probes (radioactive or non-radioactive labels); high-pressure liquid chromatography using ultraviolet detection; electro-chemiluminescence coupled with voltage-initiated chemical reaction/photon detection; and direct sequencing using radioactive or fluorescently labeled deoxyribonucleotides for the determination of the precise order of nucleotides with a DNA fragment of interest, oligo ligation assay (OLA), PCR, qPCR, DNA sequencing, fluorescence, gel electrophoresis, magnetic beads, allele specific primer extension (ASPE) and/or direct hybridization.

Generally, nucleic acid can be extracted, isolated, amplified, or analyzed by a variety of techniques such as those described by Green and Sambrook, Molecular Cloning: A Laboratory Manual (Fourth Edition), Cold Spring Harbor Laboratory Press, Woodbury, NY 2,028 pages (2012); or as described in U.S. Pat. Nos. 7,957,913; 7,776,616; 5,234,809; U.S. Pub. 2010/0285578; and U.S. Pub. 2002/0190663.

Examples of nucleic acid analysis include, but are not limited to, sequencing and DNA-protein interaction. Sequencing may be by any method known in the art. DNA sequencing techniques include classic dideoxy sequencing reactions (Sanger method) using labeled terminators or primers and gel separation in slab or capillary, and next generation sequencing methods such as sequencing by synthesis using reversibly terminated labeled nucleotides, pyrosequencing, 454 sequencing, Illumina/Solexa sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLiD sequencing. Separated molecules may be sequenced by sequential or single extension reactions using polymerases or ligases as well as by single or sequential differential hybridizations with libraries of probes.

DNA-protein interaction can be analyzed by ChIP-sequencing, also known as ChIP-seq. ChIP-seq combines chromatin immunoprecipitation (ChIP) with massively parallel DNA sequencing to identify the binding sites of DNA-associated proteins. It can be used to map global binding sites precisely for any protein of interest. Previously, ChIP-on-chip was the most common technique utilized to study these protein-DNA relations. ChIP methods are well known and described in the art.

In one embodiment, the invention provides a vector including the polynucleotide described herein, wherein the vector is an expression vector selected from the group consisting of a mammalian expression vector, a yeast expression vector, an insect expression vector, and a bacterial expression vector.

Vectors comprising polynucleotides that encode heavy chains and/or light chains of the antibodies described herein are provided. Such vectors include, but are not limited to, DNA vectors, phage vectors, viral vectors, retroviral vectors, etc. In some embodiments, a vector comprises a first polynucleotide sequence encoding a heavy chain and a second polynucleotide sequence encoding a light chain. In some embodiments, the heavy chain and light chain are expressed from the vector as two separate polypeptides. In some embodiments, the heavy chain and light chain are expressed as part of a single polypeptide, such as, for example, when the antibody is an scFv.

In some embodiments, a first vector comprises a polynucleotide that encodes a heavy chain and a second vector comprises a polynucleotide that encodes a light chain. In some embodiments, the first vector and second vector are transfected into host cells in similar amounts (such as similar molar amounts or similar mass amounts). In some embodiments, a mole- or mass-ratio of between 5:1 and 1:5 of the first vector and the second vector is transfected into host cells. In some embodiments, a mass ratio of between 1:1 and 1:5 for the vector encoding the heavy chain and the vector encoding the light chain is used. In some embodiments, a mass ratio of 1:2 for the vector encoding the heavy chain and the vector encoding the light chain is used.

In some embodiments, a vector is selected that is optimized for expression of polypeptides in CHO or CHO-derived cells, or in NSO cells. Exemplary vectors are described, e.g., in Running Deer et al., Biotechnol. Prog. 20: 880-889 (2004). In some embodiments, a vector is chosen for in vivo expression of Gal9 antibody and antigen binding fragment thereof in animals, including humans. In some such embodiments, expression of the polypeptide or polypeptides is under the control of a promoter or promoters that function in a tissue-specific manner. For example, liver-specific promoters are described, e.g., in PCT Publication No. WO 2006/076288.

The term “vector”, “expression vector”, or “plasmid DNA” is used herein to refer to a recombinant nucleic acid construct that is manipulated by human intervention. A recombinant nucleic acid construct can contain two or more nucleotide sequences that are linked in a manner such that the product is not found in a cell in nature. In particular, the two or more nucleotide sequences can be operatively linked, such as a gene encoding a protein of interest, one or more protein tags, functional domains, and the like.

Vectors suitable for use in preparation of proteins and/or protein conjugates include those selected from baculovirus, phage, plasmid, phagemid, cosmid, fosmid, bacterial artificial chromosome, viral DNA, Pl-based artificial chromosome, yeast plasmid, and yeast artificial chromosome. For example, the viral DNA vector can be selected from vaccinia, adenovirus, foul pox virus, pseudorabies, and a derivative of SV40. One type of vector is a genomic integrated vector, or “integrated vector,” which can become integrated into the chromosomal DNA of the host cell. Another type of vector is an episomal vector, e.g., a nucleic acid capable of extra-chromosomal replication. Vectors capable of directing the expression of genes to which they are operatively linked are referred to herein as “expression vectors.” Viral vectors include adenovirus, adeno-associated virus (AAV), retroviruses, lentiviruses, vaccinia virus, measles viruses, herpes viruses, and bovine papilloma virus vectors (see, Kay et al., Proc. Natl. Acad. Sci. USA 94:12744-12746 (1997) for a review of viral and non-viral vectors). Viral vectors are modified so the native tropism and pathogenicity of the virus has been altered or removed. The genome of a virus also can be modified to increase its infectivity and to accommodate packaging of the nucleic acid encoding the polypeptide of interest.

The nucleic acid construct of the present invention may be introduced into a cell to be altered thus allowing expression of the chimeric protein within the cell. A variety of methods are known in the art and suitable for introduction of nucleic acid into a cell, including viral and non-viral mediated techniques. Examples of typical non-viral mediated techniques include, but are not limited to, electroporation, calcium phosphate mediated transfer, nucleofection, sonoporation, heat shock, magnetofection, liposome mediated transfer, microinjection, microprojectile mediated transfer (nanoparticles), cationic polymer mediated transfer (DEAE-dextran, polyethylenimine, polyethylene glycol (PEG) and the like) or cell fusion. Other methods of transfection include proprietary transfection reagents such as Lipofectamine™ Dojindo Hilymax™, Fugene™, jetPEI™, Effectene™ and DreamFect™.

The nucleic acid construct of the present invention may be introduced into a host cell to be altered thus allowing expression of the chimeric protein within the cell. A variety of host cells are known in the art and suitable for chimeric proteins expression. Examples of typical cell used for transfection include, but are not limited to, a bacterial cell, a eukaryotic cell, a yeast cell, an insect cell, or a plant cell. For example, E. coli, Bacillus, Streptomyces, Pichia pastoris, Salmonella typhimurium, Drosophila S2, Spodoptera SJ9, CHO, COS (e.g., COS-7), 3T3-F442A, HeLa, HUVEC, HUAEC, NIH 3T3, Jurkat, 293, 293H, or 293F.

In various embodiments, heavy chains and/or light chains of the antibodies described herein may be expressed in prokaryotic cells, such as bacterial cells; or in eukaryotic cells, such as fungal cells (such as yeast), plant cells, insect cells, and mammalian cells. Such expression may be carried out, for example, according to procedures known in the art. Exemplary eukaryotic cells that may be used to express polypeptides include, but are not limited to, COS cells, including COS 7 cells; 293 cells, including 293-6E cells; CHO cells, including CHO-S and DG44 cells; PE.C6® cells (Crucell); and NSO cells. In some embodiments, heavy chains and/or light chains of the antibodies described herein may be expressed in yeast. See, e.g., U.S. Publication No. US 2006/0270045 A1. In some embodiments, a particular eukaryotic host cell is selected based on its ability to make desired post-translational modifications to the heavy chains and/or light chains of Gal9 antibody. For example, in some embodiments, CHO cells produce polypeptides that have a higher level of sialylation than the same polypeptide produced in 293 cells.

Introduction of one or more nucleic acids into a desired host cell may be accomplished by any method, including but not limited to, calcium phosphate transfection, DEAE-dextran mediated transfection, cationic lipid-mediated transfection, electroporation, transduction, infection, etc., Nonlimiting exemplary methods are described, e.g., in Sambrook et al., Molecular Cloning, A Laboratory Manual, 3rd ed. Cold Spring Harbor Laboratory Press (2001). Nucleic acids may be transiently or stably transfected in the desired host cells, according to any suitable method.

In some embodiments, one or more polypeptides may be produced in vivo in an animal that has been engineered or transfected with one or more nucleic acid molecules encoding the polypeptides, according to any suitable method.

Polynucleotides can be delivered to cells (e.g., a plurality of different cells or cell types including target cells or cell types and/or non-target cell types) in a vector (e.g., an expression vector). Examples of vectors include, but are not limited to, (a) non-viral vectors such as nucleic acid vectors including linear oligonucleotides and circular plasmids; artificial chromosomes such as human artificial chromosomes (HACs), yeast artificial chromosomes (YACs), and bacterial artificial chromosomes (BACs or PACs); episomal vectors; transposons (e.g., PiggyBac); and (b) viral vectors such as retroviral vectors, lentiviral vectors, adenoviral vectors, and AAV vectors. Viral vectors have several advantages for delivery of nucleic acids, including high infectivity and/or tropism for certain target cells or tissues. In some cases, a viral vector can be used to deliver the polynucleotides described herein.

In another embodiment, the invention provides a method of rescuing or promoting effector T cells proliferation, enhancing effector T cell activity, and/or of identifying and treating a subject including: (i) determining a level of Gal9 in a sample from the subject, and (ii) administering to a subject having a level Gal9 higher than a reference level of Gal9 an effective amount of any one of the isolated monoclonal antibody or antigen-binding fragment thereof described herein, thereby rescuing or promoting effector T cells proliferation, enhancing effector T cell activity, and/or of identifying and treating a subject.

An “illness associated with the suppressor activity of regulatory T lymphocytes” means any illness (not autoimmune) in which the suppressor activity of regulatory T lymphocytes plays a role, in particular by promoting the development or persistence of the illness. In particular, it has been demonstrated that the suppressor activity of regulatory T lymphocytes promotes the development of tumors. The invention therefore aims more particularly at cancers in which the suppressor activity of T lymphocytes plays a role.

In a pathological situation, Tregs may cause an inappropriate immune suppression, which could, for example, promotes tumor growth. Tregs have been associated with reducing the anti-tumoral immune responses, in particular by inappropriately inhibiting the activity of the effector T lymphocytes, thus promoting the development of numerous cancer types.

During activation, Gal9 is directly expressed by the Tregs, while it is only very weakly expressed, or not at all, by the effector T lymphocytes. Thus, targeting Gal9 by, for example, using a Gal9-specific antibody, could specifically inhibit the suppressor activity of the regulatory T lymphocytes without risking causing depletion of effector T lymphocytes. The antibodies according to the invention, directed against Gal9 and inhibiting the suppressor activity of regulatory T lymphocytes, can therefore be used in the treatment of disease or conditions associated with the suppressor activity of regulatory T lymphocytes, in particular the treatment of cancers.

The cancers treatable by the method/use of the invention include those in which the regulatory T lymphocytes exert their suppressor activity, such as those cancers in which relatively large amount of the regulatory T lymphocytes are present in the tumoral tissue or in the circulation. Expansion of the regulatory T lymphocytes (which can be measured by frequency of Tregs) is generally correlated with an increase of Tregs activation. The frequency of the regulatory T lymphocytes can be assessed by any method known in the art, for example by a flow cytometry (FACS) analysis of the intra-tumoral lymphocytes or circulating lymphocytes, or by an immuno-histological staining of the tumoral tissue.

In one aspect, the subject is diagnosed with cancer, is at risk of developing cancer or has cancer relapse.

In another aspect, the reference level of Gal9 is a level of Gal9 measured in a sample from a healthy or a control individual.

A “reference sample,” “reference cell,” or “reference tissue,” as used herein, refers to a sample, cell or tissue obtained from a source known, or believed, not to be afflicted with the disease or condition for which a method or composition of the invention is being used to identify. In one embodiment, a reference sample, reference cell or reference tissue is obtained from a healthy part of the body of the same subject or patient in whom a disease or condition is being identified using a composition or method of the invention. In one embodiment, a reference sample, reference cell or reference tissue is obtained from a healthy part of the body of at least one individual who is not the subject or patient in whom a disease or condition is being identified using a composition or method of the invention. In some embodiments, a reference sample, reference cell or reference tissue was previously obtained from a patient prior to developing a disease or condition or at an earlier stage of the disease or condition.

In some aspects, determining includes comparing the level of Gal9 in the sample to the reference level.

In one aspect, the cancer is a hematological cancer or a solid tumor. In some aspects, the hematological cancer is selected from the group consisting of acute lymphoblastic leukemia (ALL), acute myeloid leukemia (AML), chronic lymphocytic leukemia, chronic myelogenous leukemia, and hairy cell leukemia, AIDS-related lymphoma, cutaneous T-cell lymphoma, Hodgkin lymphoma, Hodgkin lymphoma, mycosis fungoides, non-Hodgkin lymphoma, primary central nervous system lymphoma, Sézary syndrome, cutaneous T-Cell lymphoma, Waldenström macroglobulinemia, diffuse large B-Cell Lymphoma (DLBCL) and multiple myeloma. In other aspects, the solid tumor is selected from the group consisting of breast cancer, head and neck cancer, lung cancer, melanoma, uveal melanoma, colorectal cancer, renal carcinoma, urothelial cancer, ovarian cancer, endometrial cancer, liver cancer, pancreatic cancer, cholangiocarcinoma, gastric cancer, gastroesophageal cancer, esophageal cancer, cervical cancer, head and neck squamous cancer, or prostate cancer.

In one aspect, the sample is a blood sample, a plasma sample, a serum sample, a tumor biopsy sample, or a bone-marrow (BM)-derived mononuclear cell (MNC) sample.

In one aspect, the method further includes administering to the patient a chemotherapeutic agent, an anti-angiogenesis agent, a growth inhibitory agent, an immune-oncology agent, a checkpoint inhibitor, and/or an anti-neoplastic composition. In some aspects, the checkpoint inhibitor is an anti-PD-1. In other aspects, the immune-oncology agent is an anti-glucocorticoid-induced tumor necrosis factor receptor family-related protein (GITR) antibody.

### Additional Embodiments

Embodiment 1. An isolated monoclonal antibody or an antigen-binding fragment thereof comprising:
   (a) a HCVR comprising (i) a HCVR CDR1 sequence having the amino acid sequence GYX₁FX₂X₃YTIH (SEQ ID NO:1), wherein X₁ is selected from T, E, P, or A; X₂ is selected from T, G, or S; and X₃ is selected from E, S, or D; (ii) a HCVR CDR2 sequence having the amino acid sequence WFYPGSGSTX₄YAQKFQG (SEQ ID NO:8), wherein X₄ is selected from E, W, or V; and (iii) a HCVR CDR3 sequence having the amino acid sequence HGGYDGFDY (SEQ ID NO:12); and
   (b) a LCVR comprising, (i) a LCVR CDR1 sequence having the amino acid sequence KSSX₅X₆X₇LX₈X₉X₁₀X₁₁X₁₂X₁₃NX₁₄LA (SEQ ID NO:13), wherein X₅ is selected from Q or R; X₆ is selected from S or N; X₇ is selected from L, V, or I; X₈ is selected from Y or W; X₉ is selected from S or P; X₁₀ is selected from N, S, P, or A; X₁₁ is selected from N or H; X₁₂ is selected from Q, N, or Y; X₁₃ is selected from K or R; and X₁₄ is selected from Y or H; (ii) a LCVR CDR2 sequence having the amino acid sequence WX₁₅SX₁₆RX₁₇X₁₈, wherein X₁₅ is selected from A or G; X₁₆ is selected from T, N, M, or A; X₁₇ is selected from G or E; and X₁₈ is selected from S, E, Y, P, or T; and (iii) a LCVR CDR3 sequence having the amino acid sequence QQYYX₁₉X₂₀PFT (SEQ ID NO:30), wherein X₁₉ is selected from S or F; and X₂₀ is selected from Y or F,
wherein the antibody or antigen-binding fragment thereof specifically binds Galectin-9 (Gal9), with the proviso that the antibody does not comprise HCVR CDR1 sequence GYTFTEYTIH (SEQ ID NO:2), HCVR CDR2 sequence WFYPGSGSTEYAQKFQG (SEQ ID NO:9), HCVR CDR3 sequence HGGYDGFDY (SEQ ID NO:12), LCVR CDR1 sequence KSSQSLLYSNNQKNYLA (SEQ ID NO:14), LCVR CDR2 sequence WASTRGS (SEQ ID NO:22), and LCVR CDR3 sequence QQYYSYPFT (SEQ ID NO:31).
Embodiment 2. The isolated monoclonal antibody or an antigen-binding fragment thereof of embodiment 1, comprising:
   (a) a HCVR comprising (i) a HCVR CDR1 sequence having the amino acid sequence GYX₁FX₂X₃YTIH (SEQ ID NO:1), wherein X₁ is selected from T or E, and X₂ is selected from T or G, and X₃ is E; (ii) a HCVR CDR2 sequence having the amino acid sequence WFYPGSGSTX₄YAQKFQG (SEQ ID NO:8), wherein X₄ is selected from W or V; and (iii) a HCVR CDR3 sequence having the amino acid sequence HGGYDGFDY (SEQ ID NO:12); and
   (b) a LCVR comprising (i) a LCVR CDR1 sequence having the amino acid sequence KSSX₅X₆X₇LX₈X₉X₁₀X₁₁X₁₂X₁₃N X₁₄LA (SEQ ID NO:13), wherein X₅ is Q, X₆ is S, X₇ is L, X₈ is Y, X₉ is selected from S or P, X₁₀ is N, X₁₁ is N, X₁₂ is Q, X₁₃ is K; and X₁₄ is Y; (ii) a LCVR CDR2 sequence having the amino acid sequence WX₁₅SX₁₆RX₁₇X₁₈, wherein X₁₅ is A, X₁₆ is T, X₁₇ is G, and X₁₈ is selected from S or E, and (iii) a LCVR CDR3 sequence having the amino acid sequence QQYYX₁₉X₂₀PFT (SEQ ID NO:30), wherein X₁₉ is S, and X₂₀ is selected from Y or F,
wherein the antibody or antigen-binding fragment thereof specifically binds Gal9.
Embodiment 3. The isolated monoclonal antibody or an antigen-binding fragment thereof of embodiment 1 or 2, comprising:
   (A) a HCVR, comprising a HCVR CDR1 sequence of SEQ ID NO:3, a HCVR CDR2 sequence of SEQ ID NO:10, and a HCVR CDR3 sequence of SEQ ID NO:12; and a LCVR, including a LCVR CDR1 sequence of SEQ ID NO:15, a LCVR CDR2 sequence of SEQ ID NO:23, and a LCVR CDR3 sequence of SEQ ID NO:32; or
   (B) a HCVR, comprising a HCVR CDR1 sequence of SEQ ID NO:4, a HCVR CDR2 sequence of SEQ ID NO:11, and a HCVR CDR3 sequence of SEQ ID NO:12; and, a light chain variable region (LCVR), including a LCVR CDR1 sequence of SEQ ID NO:14, a LCVR CDR2 sequence of SEQ ID NO:24, and a LCVR CDR3 sequence of SEQ ID NO:31,
wherein the antibody or antigen-binding fragment thereof specifically binds Gal9.
Embodiment 4. The isolated monoclonal antibody or an antigen-binding fragment thereof of embodiment 1, comprising:
   (a) a HCVR, comprising (i) a HCVR CDR1 sequence having the amino acid sequence GYX₁FX₂X₃YTIH (SEQ ID NO:1), wherein X₁ is selected from T, P, or A; X₂ is selected from T, S, or G; and X₃ is selected from S or D; (ii) a HCVR CDR2 sequence having the amino acid sequence WFYPGSGSTX₄YAQKFQG (SEQ ID NO:8), wherein X₄ is E; and (iii) a HCVR CDR3 sequence having the amino acid sequence HGGYDGFDY (SEQ ID NO:12); and
   (b) a LCVR comprising: (i) a LCVR CDR1 sequence having the amino acid sequence KSSX₅X₆X₇LX₈X₉X₁₀X₁₁X₁₂X₁₃N X₁₄LA (SEQ ID NO:13), wherein X₅ is selected from Q or R; X₆ is selected from S or N; X₇ is selected from L, V, or I; X₈ is selected from Y or W; X₉ is S, X₁₀ is selected from S, P, or A; X₁₁ is selected from N or H; X₁₂ is N or Y; X₁₃ is K or R; and X₁₄ is Y or H; (ii) a LCVR CDR2 sequence having the amino acid sequence WX₁₅SX₁₆RX₁₇X₁₈, wherein X₁₅ is selected from A or G; X₁₆ is selected from T, N, M, or A; X₁₇ is selected from G or E; and X₁₈ is selected from S, Y, P, or T; and (iii) a LCVR CDR3 sequence having the amino acid sequence QQYYX₁₉X₂₀PFT (SEQ ID NO:30), wherein X₁₉ is F, and X₂₀ is Y,
wherein the antibody or antigen-binding fragment thereof specifically binds Gal9.
Embodiment 5. The isolated monoclonal antibody or an antigen-binding fragment thereof of embodiment 1 or 4, comprising:
   (C) a HCVR, comprising a HCVR CDR1 sequence of SEQ ID NO:5, a HCVR CDR2 sequence of SEQ ID NO:9, and a HCVR CDR3 sequence of SEQ ID NO:12; and, a light chain variable region (LCVR), comprising a LCVR CDR1 sequence of SEQ ID NO:16, a LCVR CDR2 sequence of SEQ ID NO:25, and a LCVR CDR3 sequence of SEQ ID NO:33; or
   (D) a HCVR, comprising a HCVR CDR1 sequence of SEQ ID NO:6, a HCVR CDR2 sequence of SEQ ID NO:9, and a HCVR CDR3 sequence of SEQ ID NO:12; and a LCVR, comprising a LCVR CDR1 sequence of SEQ ID NO:17, a LCVR CDR2 sequence of SEQ ID NO:26, and a LCVR CDR3 sequence of SEQ ID NO:33, or
   (E) a HCVR, comprising a HCVR CDR1 sequence of SEQ ID NO:7, a HCVR CDR2 sequence of SEQ ID NO:9, and a HCVR CDR3 sequence of SEQ ID NO:12; and a LCVR, comprising a LCVR CDR1 sequence of SEQ ID NO:18, a LCVR CDR2 sequence of SEQ ID NO:27, and a LCVR CDR3 sequence of SEQ ID NO:33, or
   (F) a HCVR, comprising a HCVR CDR1 sequence of SEQ ID NO:7, a HCVR CDR2 sequence of SEQ ID NO:9, and a HCVR CDR3 sequence of SEQ ID NO:12; and a LCVR, comprising a LCVR CDR1 sequence of SEQ ID NO:19, a LCVR CDR2 sequence of SEQ ID NO:28, and a LCVR CDR3 sequence of SEQ ID NO:33, or
   (G) a HCVR, comprising a HCVR CDR1 sequence of SEQ ID NO:7, a HCVR CDR2 sequence of SEQ ID NO:9, and a HCVR CDR3 sequence of SEQ ID NO:12; and a LCVR, comprising a LCVR CDR1 sequence of SEQ ID NO:20, a LCVR CDR2 sequence of SEQ ID NO:29, and a LCVR CDR3 sequence of SEQ ID NO:33,
wherein the antibody or antigen-binding fragment thereof specifically binds Gal9.
Embodiment 6. The isolated monoclonal antibody or an antigen-binding fragment thereof of embodiment 1, comprising:
   (a) a HCVR, comprising (i) a HCVR CDR1 sequence having the amino acid sequence GYX₁FX₂X₃YTIH (SEQ ID NO:1), wherein X₁ is selected from E or P; X₂ is selected from T or S; and X₃ selected from E or S; (ii) a HCVR CDR2 sequence having the amino acid sequence WF YPGSGSTX₄YAQKFQG (SEQ ID NO:8), wherein X₄ is selected from W or E; and (iii) a HCVR CDR3 sequence having the amino acid sequence HGGYDGFDY (SEQ ID NO:12), and
   (b) a light chain variable region (LCVR), comprising: (i) a LCVR CDR1 sequence having the amino acid sequence KSSX₅X₆X₇LX₈X₉X₁₀X₁₁X₁₂X₁₃NX₁₄LA (SEQ ID NO:13), wherein X₅ is Q, X₆ is selected from S or N, X₇ is selected from L or V, X₈ is selected from Y or W, X₉ is selected from P or S, X₁₀ is selected from N or S, X₁₁ is N, X₁₂ is selected from Q or N, X₁₃ is K; and X₁₄ is Y; (ii) a LCVR CDR2 sequence having the amino acid sequence WX₁₅SX₁₆RX₁₇X₁₈, wherein X₁₅ is selected from A or G; X₁₆ is selected from T or N; X₁₇ is selected from G or E, and X₁₈ is S; and (iii) a LCVR CDR3 sequence having the amino acid sequence QQYYX₁₉X₂₀PFT (SEQ ID NO:30), wherein X₁₉ is selected from S or F; and X₂₀ is selected from Y or F,
wherein the antibody or antigen-binding fragment thereof specifically binds Gal9.
Embodiment 7. The isolated monoclonal antibody or an antigen-binding fragment thereof of embodiment 1 or 6, comprising:
   (A) a HCVR, comprising a HCVR CDR1 sequence of SEQ ID NO:3, a HCVR CDR2 sequence of SEQ ID NO:10, and a HCVR CDR3 sequence of SEQ ID NO:12; and, a LCVR, including a LCVR CDR1 sequence of SEQ ID NO:15, a LCVR CDR2 sequence of SEQ ID NO:23, and a LCVR CDR3 sequence of SEQ ID NO:32; or
   (C) a HCVR, comprising a HCVR CDR1 sequence of SEQ ID NO:5, a HCVR CDR2 sequence of SEQ ID NO:9, and a HCVR CDR3 sequence of SEQ ID NO:12; and, a light chain variable region (LCVR), comprising a LCVR CDR1 sequence of SEQ ID NO:16, a LCVR CDR2 sequence of SEQ ID NO:25, and a LCVR CDR3 sequence of SEQ ID NO:33,
wherein the antibody or antigen-binding fragment thereof specifically binds Gal9.
Embodiment 8. An isolated monoclonal antibody or an antigen-binding fragment thereof comprising: a HCVR, comprising a HCVR CDR1 sequence of SEQ ID NO:3, a HCVR CDR2 sequence of SEQ ID NO:10, and a HCVR CDR3 sequence of SEQ ID NO:12; and a LCVR, including a LCVR CDR1 sequence of SEQ ID NO:15, a LCVR CDR2 sequence of SEQ ID NO:23, and a LCVR CDR3 sequence of SEQ ID NO:32, wherein the antibody or antigen-binding fragment thereof specifically binds Gal9.
Embodiment 9. The isolated monoclonal antibody or antigen-binding fragment thereof of embodiment 8, wherein: the HCVR sequence of the antibody or antigen-binding fragment thereof further comprises a HFR3 sequence of SEQ ID NO:68, and optionally further includes a HFR1 sequence of SEQ ID NO:66, a HFR2 sequence of SEQ ID NO:67, and/or a HFR4 sequence of SEQ ID NO:71.
Embodiment 10. The isolated monoclonal antibody or antigen-binding fragment thereof of embodiment 8, wherein: the HCVR sequence of the antibody or antigen-binding fragment thereof further comprises a HFR3 sequence of SEQ ID NO:69, and optionally further includes a HFR1 sequence of SEQ ID NO:66, a HFR2 sequence of SEQ ID NO:67, and/or a HFR4 sequence of SEQ ID NO:71.
Embodiment 11. The isolated monoclonal antibody or antigen-binding fragment thereof of embodiment 8, wherein: the HCVR sequence of the antibody or antigen-binding fragment thereof further comprises a HFR3 sequence of SEQ ID NO:70, and optionally further includes a HFR1 sequence of SEQ ID NO:66, a HFR2 sequence of SEQ ID NO:67, and/or a HFR4 sequence of SEQ ID NO:71.
Embodiment 12. The isolated monoclonal antibody or antigen-binding fragment thereof of embodiment 8, wherein: the HCVR sequence of the antibody or antigen-binding fragment thereof further comprises a HFR3 sequence of SEQ ID NO:76, and optionally further includes a HFR1 sequence of SEQ ID NO:66, a HFR2 sequence of SEQ ID NO:67, and/or a HFR4 sequence of SEQ ID NO:71.
Embodiment 13. The isolated monoclonal antibody or antigen-binding fragment thereof of embodiment 8, wherein: the HCVR sequence of the antibody or antigen-binding fragment thereof further comprises a HFR3 sequence of SEQ ID NO:77, and optionally further includes a HFR1 sequence of SEQ ID NO:66, a HFR2 sequence of SEQ ID NO:67, and/or a HFR4 sequence of SEQ ID NO:71.
Embodiment 14. The isolated monoclonal antibody or antigen-binding fragment thereof of any one of embodiments 8-13, wherein: the LCVR sequence of the antibody or antigen-binding fragment thereof of further includes a LFR1 sequence of SEQ ID NO:72, a LFR2 sequence of SEQ ID NO:73, a LFR3 sequence of SEQ ID NO:74, and/or a LFR4 sequence of SEQ ID NO:75.
Embodiment 15. The isolated monoclonal antibody or antigen-binding fragment thereof of embodiment 8 wherein: the HCVR sequence is SEQ ID NO:39 and/or the LCVR sequence is SEQ ID NO:41.
Embodiment 16. The isolated monoclonal antibody or antigen-binding fragment thereof of embodiments 8 or 5, wherein: the HCVR sequence is SEQ ID NO:39 and the LCVR sequence of SEQ ID NO:41.
Embodiment 17. An isolated monoclonal antibody or an antigen-binding fragment thereof comprising: a HCVR, comprising a HCVR CDR1 sequence of SEQ ID NO:4, a HCVR CDR2 sequence of SEQ ID NO:11, and a HCVR CDR3 sequence of SEQ ID NO:12; and, a light chain variable region (LCVR), including a LCVR CDR1 sequence of SEQ ID NO:14, a LCVR CDR2 sequence of SEQ ID NO:24, and a LCVR CDR3 sequence of SEQ ID NO:31, wherein the antibody or antigen-binding fragment thereof specifically binds Galectin-9 (Gal9).
Embodiment 18. The isolated monoclonal antibody or antigen-binding fragment thereof of embodiment 17, wherein: the HCVR sequence of the antibody or antigen-binding fragment thereof further comprises a HFR3 sequence of SEQ ID NO:68, and optionally further includes a HFR1 sequence of SEQ ID NO:66, a HFR2 sequence of SEQ ID NO:67, and/or a HFR4 sequence of SEQ ID NO:71.
Embodiment 19. The isolated monoclonal antibody or antigen-binding fragment thereof of embodiment 17, wherein: the HCVR sequence of the antibody or antigen-binding fragment thereof further comprises a HFR3 sequence of SEQ ID NO:69, and optionally further includes a HFR1 sequence of SEQ ID NO:66, a HFR2 sequence of SEQ ID NO:67, and/or a HFR4 sequence of SEQ ID NO:71.
Embodiment 20. The isolated monoclonal antibody or antigen-binding fragment thereof of embodiment 17, wherein: the HCVR sequence of the antibody or antigen-binding fragment thereof further comprises a HFR3 sequence of SEQ ID NO:70, and optionally further includes a HFR1 sequence of SEQ ID NO:66, a HFR2 sequence of SEQ ID NO:67, and/or a HFR4 sequence of SEQ ID NO:71.
Embodiment 21. The isolated monoclonal antibody or antigen-binding fragment thereof of embodiment 17, wherein: the HCVR sequence of the antibody or antigen-binding fragment thereof further comprises a HFR3 sequence of SEQ ID NO:76, and optionally further includes a HFR1 sequence of SEQ ID NO:66, a HFR2 sequence of SEQ ID NO:67, and/or a HFR4 sequence of SEQ ID NO:71.
Embodiment 22. The isolated monoclonal antibody or antigen-binding fragment thereof of embodiment 17, wherein: the HCVR sequence of the antibody or antigen-binding fragment thereof further comprises a HFR3 sequence of SEQ ID NO:77, and optionally further includes a HFR1 sequence of SEQ ID NO:66, a HFR2 sequence of SEQ ID NO:67, and/or a HFR4 sequence of SEQ ID NO:71.
Embodiment 23. The isolated monoclonal antibody or antigen-binding fragment thereof of any one of embodiments 17-22, wherein: the LCVR sequence of the antibody or antigen-binding fragment thereof of further includes a LFR1 sequence of SEQ ID NO:72, a LFR2 sequence of SEQ ID NO:73, a LFR3 sequence of SEQ ID NO:74, and/or a LFR4 sequence of SEQ ID NO:75.
Embodiment 24. The isolated monoclonal antibody or antigen-binding fragment thereof of embodiment 17, wherein the HCVR sequence is SEQ ID NO:43 and/or the LCVR sequence is SEQ ID NO:45.
Embodiment 25. The isolated monoclonal antibody or antigen-binding fragment thereof embodiments 17 or 24, wherein the HCVR sequence of SEQ ID NO:43 and the LCVR sequence of SEQ ID NO:45.
Embodiment 26. An isolated monoclonal antibody or an antigen-binding fragment thereof comprising: a HCVR, comprising a HCVR CDR1 sequence of SEQ ID NO:5, a HCVR CDR2 sequence of SEQ ID NO:9, and a HCVR CDR3 sequence of SEQ ID NO:12; and a light chain variable region (LCVR), comprising a LCVR CDR1 sequence of SEQ ID NO:16, a LCVR CDR2 sequence of SEQ ID NO:25, and a LCVR CDR3 sequence of SEQ ID NO:33, wherein the antibody or antigen-binding fragment thereof specifically binds Gal9.
Embodiment 27. The isolated monoclonal antibody or antigen-binding fragment thereof of embodiment 26, wherein: the HCVR sequence of the antibody or antigen-binding fragment thereof further comprises a HFR3 sequence of SEQ ID NO:68, and optionally further includes a HFR1 sequence of SEQ ID NO:66, a HFR2 sequence of SEQ ID NO:67, and/or a HFR4 sequence of SEQ ID NO:71.
Embodiment 28. The isolated monoclonal antibody or antigen-binding fragment thereof of embodiment 26, wherein: the HCVR sequence of the antibody or antigen-binding fragment thereof further comprises a HFR3 sequence of SEQ ID NO:69, and optionally further includes a HFR1 sequence of SEQ ID NO:66, a HFR2 sequence of SEQ ID NO:67, and/or a HFR4 sequence of SEQ ID NO:71.
Embodiment 29. The isolated monoclonal antibody or antigen-binding fragment thereof of embodiment 26, wherein: the HCVR sequence of the antibody or antigen-binding fragment thereof further comprises a HFR3 sequence of SEQ ID NO:70, and optionally further includes a HFR1 sequence of SEQ ID NO:66, a HFR2 sequence of SEQ ID NO:67, and/or a HFR4 sequence of SEQ ID NO:71.
Embodiment 30. The isolated monoclonal antibody or antigen-binding fragment thereof of embodiment 26, wherein: the HCVR sequence of the antibody or antigen-binding fragment thereof further comprises a HFR3 sequence of SEQ ID NO:76, and optionally further includes a HFR1 sequence of SEQ ID NO:66, a HFR2 sequence of SEQ ID NO:67, and/or a HFR4 sequence of SEQ ID NO:71.
Embodiment 31. The isolated monoclonal antibody or antigen-binding fragment thereof of embodiment 26, wherein: the HCVR sequence of the antibody or antigen-binding fragment thereof further comprises a HFR3 sequence of SEQ ID NO:77, and optionally further includes a HFR1 sequence of SEQ ID NO:66, a HFR2 sequence of SEQ ID NO:67, and/or a HFR4 sequence of SEQ ID NO:71.
Embodiment 32. The isolated monoclonal antibody or antigen-binding fragment thereof of any one of embodiments 26-31, wherein: the LCVR sequence of the antibody or antigen-binding fragment thereof of further includes a LFR1 sequence of SEQ ID NO:72, a LFR2 sequence of SEQ ID NO:73, a LFR3 sequence of SEQ ID NO:74, and/or a LFR4 sequence of SEQ ID NO:75.
Embodiment 33. The isolated monoclonal antibody or antigen-binding fragment thereof of embodiment 26, wherein the HCVR sequence is SEQ ID NO:47 and/or the LCVR sequence is SEQ ID NO:49.
Embodiment 34. The isolated monoclonal antibody or antigen-binding fragment thereof embodiments 26 or 33, wherein the HCVR sequence of SEQ ID NO:47 and the LCVR sequence of SEQ ID NO:49.
Embodiment 35. An isolated monoclonal antibody or an antigen-binding fragment thereof comprising: a HCVR, comprising a HCVR CDR1 sequence of SEQ ID NO:7, a HCVR CDR2 sequence of SEQ ID NO:9, and a HCVR CDR3 sequence of SEQ ID NO:12; and a LCVR, comprising a LCVR CDR1 sequence of SEQ ID NO:18, a LCVR CDR2 sequence of SEQ ID NO:27, and a LCVR CDR3 sequence of SEQ ID NO:33, wherein the antibody or antigen-binding fragment thereof specifically binds Galectin-9 (Gal9).
Embodiment 36. The isolated monoclonal antibody or antigen-binding fragment thereof of embodiment 35, wherein: the HCVR sequence of the antibody or antigen-binding fragment thereof further comprises a HFR3 sequence of SEQ ID NO:68, and optionally further includes a HFR1 sequence of SEQ ID NO:66, a HFR2 sequence of SEQ ID NO:67, and/or a HFR4 sequence of SEQ ID NO:71.
Embodiment 37. The isolated monoclonal antibody or antigen-binding fragment thereof of embodiment 35, wherein: the HCVR sequence of the antibody or antigen-binding fragment thereof further comprises a HFR3 sequence of SEQ ID NO:69, and optionally further includes a HFR1 sequence of SEQ ID NO:66, a HFR2 sequence of SEQ ID NO:67, and/or a HFR4 sequence of SEQ ID NO:71.
Embodiment 38. The isolated monoclonal antibody or antigen-binding fragment thereof of embodiment 35, wherein: the HCVR sequence of the antibody or antigen-binding fragment thereof further comprises a HFR3 sequence of SEQ ID NO:70, and optionally further includes a HFR1 sequence of SEQ ID NO:66, a HFR2 sequence of SEQ ID NO:67, and/or a HFR4 sequence of SEQ ID NO:71.
Embodiment 39. The isolated monoclonal antibody or antigen-binding fragment thereof of embodiment 35, wherein: the HCVR sequence of the antibody or antigen-binding fragment thereof further comprises a HFR3 sequence of SEQ ID NO:76, and optionally further includes a HFR1 sequence of SEQ ID NO:66, a HFR2 sequence of SEQ ID NO:67, and/or a HFR4 sequence of SEQ ID NO:71.
Embodiment 40. The isolated monoclonal antibody or antigen-binding fragment thereof of embodiment 35, wherein: the HCVR sequence of the antibody or antigen-binding fragment thereof further comprises a HFR3 sequence of SEQ ID NO:77, and optionally further includes a HFR1 sequence of SEQ ID NO:66, a HFR2 sequence of SEQ ID NO:67, and/or a HFR4 sequence of SEQ ID NO:71.
Embodiment 41. The isolated monoclonal antibody or antigen-binding fragment thereof of any one of embodiments 35-40, wherein: the LCVR sequence of the antibody or antigen-binding fragment thereof of further includes a LFR1 sequence of SEQ ID NO:72, a LFR2 sequence of SEQ ID NO:73, a LFR3 sequence of SEQ ID NO:74, and/or a LFR4 sequence of SEQ ID NO:75.
Embodiment 42. The isolated monoclonal antibody or antigen-binding fragment thereof of embodiment 35, wherein the HCVR sequence is SEQ ID NO:51 and/or the LCVR sequence is SEQ ID NO:53.
Embodiment 43. The isolated monoclonal antibody or antigen-binding fragment thereof embodiments 35 or 42, wherein the HCVR sequence of SEQ ID NO:51 and the LCVR sequence of SEQ ID NO:53.
Embodiment 44. An isolated monoclonal antibody or an antigen-binding fragment thereof comprising: a HCVR, comprising a HCVR CDR1 sequence of SEQ ID NO:7, a HCVR CDR2 sequence of SEQ ID NO:9, and a HCVR CDR3 sequence of SEQ ID NO:12; and a LCVR, comprising a LCVR CDR1 sequence of SEQ ID NO:18, a LCVR CDR2 sequence of SEQ ID NO:27, and a LCVR CDR3 sequence of SEQ ID NO:33, wherein the antibody or antigen-binding fragment thereof specifically binds Galectin-9 (Gal9).
Embodiment 45. The isolated monoclonal antibody or antigen-binding fragment thereof of embodiment 44, wherein: the HCVR sequence of the antibody or antigen-binding fragment thereof further comprises a HFR3 sequence of SEQ ID NO:68, and optionally further includes a HFR1 sequence of SEQ ID NO:66, a HFR2 sequence of SEQ ID NO:67, and/or a HFR4 sequence of SEQ ID NO:71.
Embodiment 46. The isolated monoclonal antibody or antigen-binding fragment thereof of embodiment 44, wherein: the HCVR sequence of the antibody or antigen-binding fragment thereof further comprises a HFR3 sequence of SEQ ID NO:69, and optionally further includes a HFR1 sequence of SEQ ID NO:66, a HFR2 sequence of SEQ ID NO:67, and/or a HFR4 sequence of SEQ ID NO:71
Embodiment 47. The isolated monoclonal antibody or antigen-binding fragment thereof of embodiment 44, wherein: the HCVR sequence of the antibody or antigen-binding fragment thereof further comprises a HFR3 sequence of SEQ ID NO:70, and optionally further includes a HFR1 sequence of SEQ ID NO:66, a HFR2 sequence of SEQ ID NO:67, and/or a HFR4 sequence of SEQ ID NO:71.
Embodiment 48. The isolated monoclonal antibody or antigen-binding fragment thereof of embodiment 44, wherein: the HCVR sequence of the antibody or antigen-binding fragment thereof further comprises a HFR3 sequence of SEQ ID NO:76, and optionally further includes a HFR1 sequence of SEQ ID NO:66, a HFR2 sequence of SEQ ID NO:67, and/or a HFR4 sequence of SEQ ID NO:71.
Embodiment 49. The isolated monoclonal antibody or antigen-binding fragment thereof of embodiment 44, wherein: the HCVR sequence of the antibody or antigen-binding fragment thereof further comprises a HFR3 sequence of SEQ ID NO:77, and optionally further includes a HFR1 sequence of SEQ ID NO:66, a HFR2 sequence of SEQ ID NO:67, and/or a HFR4 sequence of SEQ ID NO:71.
Embodiment 50. The isolated monoclonal antibody or antigen-binding fragment thereof of any one of embodiments 44-49, wherein: the LCVR sequence of the antibody or antigen-binding fragment thereof of further includes a LFR1 sequence of SEQ ID NO:72, a LFR2 sequence of SEQ ID NO:73, a LFR3 sequence of SEQ ID NO:74, and/or a LFR4 sequence of SEQ ID NO:75.
Embodiment 51. The isolated monoclonal antibody or antigen-binding fragment thereof of embodiment 44, wherein the HCVR sequence is SEQ ID NO:55 and/or the LCVR sequence is SEQ ID NO:57.
Embodiment 52. The isolated monoclonal antibody or antigen-binding fragment thereof embodiments 44 or 51, wherein the HCVR sequence of SEQ ID NO:55 and the LCVR sequence of SEQ ID NO:57.
Embodiment 53. An isolated monoclonal antibody or an antigen-binding fragment thereof comprising: a HCVR, comprising a HCVR CDR1 sequence of SEQ ID NO:7, a HCVR CDR2 sequence of SEQ ID NO:9, and a HCVR CDR3 sequence of SEQ ID NO:12; and a LCVR, comprising a LCVR CDR1 sequence of SEQ ID NO:19, a LCVR CDR2 sequence of SEQ ID NO:28, and a LCVR CDR3 sequence of SEQ ID NO:33, wherein the antibody or antigen-binding fragment thereof specifically binds Galectin-9 (Gal9).
Embodiment 54. The isolated monoclonal antibody or antigen-binding fragment thereof of embodiment 53, wherein: the HCVR sequence of the antibody or antigen-binding fragment thereof further comprises a HFR3 sequence of SEQ ID NO:68, and optionally further includes a HFR1 sequence of SEQ ID NO:66, a HFR2 sequence of SEQ ID NO:67, and/or a HFR4 sequence of SEQ ID NO:71.
Embodiment 55. The isolated monoclonal antibody or antigen-binding fragment thereof of embodiment 53, wherein: the HCVR sequence of the antibody or antigen-binding fragment thereof further comprises a HFR3 sequence of SEQ ID NO:69, and optionally further includes a HFR1 sequence of SEQ ID NO:66, a HFR2 sequence of SEQ ID NO:67, and/or a HFR4 sequence of SEQ ID NO:71.
Embodiment 56. The isolated monoclonal antibody or antigen-binding fragment thereof of embodiment 53, wherein: the HCVR sequence of the antibody or antigen-binding fragment thereof further comprises a HFR3 sequence of SEQ ID NO:70, and optionally further includes a HFR1 sequence of SEQ ID NO:66, a HFR2 sequence of SEQ ID NO:67, and/or a HFR4 sequence of SEQ ID NO:71.
Embodiment 57. The isolated monoclonal antibody or antigen-binding fragment thereof of embodiment 53, wherein: the HCVR sequence of the antibody or antigen-binding fragment thereof further comprises a HFR3 sequence of SEQ ID NO:76, and optionally further includes a HFR1 sequence of SEQ ID NO:66, a HFR2 sequence of SEQ ID NO:67, and/or a HFR4 sequence of SEQ ID NO:71.
Embodiment 58. The isolated monoclonal antibody or antigen-binding fragment thereof of embodiment 53, wherein: the HCVR sequence of the antibody or antigen-binding fragment thereof further comprises a HFR3 sequence of SEQ ID NO:77, and optionally further includes a HFR1 sequence of SEQ ID NO:66, a HFR2 sequence of SEQ ID NO:67, and/or a HFR4 sequence of SEQ ID NO:71.
Embodiment 59. The isolated monoclonal antibody or antigen-binding fragment thereof of any one of embodiments 53-58, wherein: the LCVR sequence of the antibody or antigen-binding fragment thereof of further includes a LFR1 sequence of SEQ ID NO:72, a LFR2 sequence of SEQ ID NO:73, a LFR3 sequence of SEQ ID NO:74, and/or a LFR4 sequence of SEQ ID NO:75.
Embodiment 60. The isolated monoclonal antibody or antigen-binding fragment thereof of embodiment 53, wherein the HCVR sequence is SEQ ID NO:59 and/or the LCVR sequence is SEQ ID NO:61.
Embodiment 61. The isolated monoclonal antibody or antigen-binding fragment thereof embodiments 53 or 60, wherein the HCVR sequence of SEQ ID NO:59 and the LCVR sequence of SEQ ID NO:61.
Embodiment 62. An isolated monoclonal antibody or an antigen-binding fragment thereof comprising: a HCVR, comprising a HCVR CDR1 sequence of SEQ ID NO:7, a HCVR CDR2 sequence of SEQ ID NO:9, and a HCVR CDR3 sequence of SEQ ID NO:12; and a LCVR, comprising a LCVR CDR1 sequence of SEQ ID NO:20, a LCVR CDR2 sequence of SEQ ID NO:29, and a LCVR CDR3 sequence of SEQ ID NO:33, wherein the antibody or antigen-binding fragment thereof specifically binds Galectin-9 (Gal9).
Embodiment 63. The isolated monoclonal antibody or antigen-binding fragment thereof of embodiment 62, wherein: the HCVR sequence of the antibody or antigen-binding fragment thereof further comprises a HFR3 sequence of SEQ ID NO:68, and optionally further includes a HFR1 sequence of SEQ ID NO:66, a HFR2 sequence of SEQ ID NO:67, and/or a HFR4 sequence of SEQ ID NO:71.
Embodiment 64. The isolated monoclonal antibody or antigen-binding fragment thereof of embodiment 62, wherein: the HCVR sequence of the antibody or antigen-binding fragment thereof further comprises a HFR3 sequence of SEQ ID NO:69, and optionally further includes a HFR1 sequence of SEQ ID NO:66, a HFR2 sequence of SEQ ID NO:67, and/or a HFR4 sequence of SEQ ID NO:71.
Embodiment 65. The isolated monoclonal antibody or antigen-binding fragment thereof of embodiment 62, wherein: the HCVR sequence of the antibody or antigen-binding fragment thereof further comprises a HFR3 sequence of SEQ ID NO:70, and optionally further includes a HFR1 sequence of SEQ ID NO:66, a HFR2 sequence of SEQ ID NO:67, and/or a HFR4 sequence of SEQ ID NO:71.
Embodiment 66. The isolated monoclonal antibody or antigen-binding fragment thereof of embodiment 62, wherein: the HCVR sequence of the antibody or antigen-binding fragment thereof further comprises a HFR3 sequence of SEQ ID NO:76, and optionally further includes a HFR1 sequence of SEQ ID NO:66, a HFR2 sequence of SEQ ID NO:67, and/or a HFR4 sequence of SEQ ID NO:71.
Embodiment 67. The isolated monoclonal antibody or antigen-binding fragment thereof of embodiment 62, wherein: the HCVR sequence of the antibody or antigen-binding fragment thereof further comprises a HFR3 sequence of SEQ ID NO:77, and optionally further includes a HFR1 sequence of SEQ ID NO:66, a HFR2 sequence of SEQ ID NO:67, and/or a HFR4 sequence of SEQ ID NO:71.
Embodiment 68. The isolated monoclonal antibody or antigen-binding fragment thereof of any one of embodiments 62-67, wherein: the LCVR sequence of the antibody or antigen-binding fragment thereof of further includes a LFR1 sequence of SEQ ID NO:72, a LFR2 sequence of SEQ ID NO:73, a LFR3 sequence of SEQ ID NO:74, and/or a LFR4 sequence of SEQ ID NO:75.
Embodiment 69. The isolated monoclonal antibody or antigen-binding fragment thereof of embodiment 62, wherein the HCVR sequence is SEQ ID NO:63 and/or the LCVR sequence is SEQ ID NO:65.
Embodiment 70. The isolated monoclonal antibody or antigen-binding fragment thereof embodiments 62 or 69, wherein the HCVR sequence of SEQ ID NO:63 and the LCVR sequence of SEQ ID NO:65.
Embodiment 71. The isolated monoclonal antibody or antigen-binding fragment thereof according to any one of embodiments 1-71, wherein the antibody is a humanized antibody.
Embodiment 72. The isolated monoclonal antibody or antigen-binding fragment thereof according to any one of embodiments 1-71, wherein the antigen-binding fragment thereof is an Fab, Fab′, F(ab′)₂, F_{d}, single chain Fv or scFv, disulfide linked Fᵥ, V-NAR domain, IgNar, intrabody, IgGACH₂, minibody, F(ab′)₃, tetrabody, triabody, diabody, domain antibody (dAb), DVD-Ig, Fcab, mAb₂, (scFv)₂, tandem scFv, DART®, TandAb, nanobody or scFv-Fc.
Embodiment 73. An isolated monoclonal antibody comprising the heavy chain sequence of SEQ ID NO:38 and the light chain sequence of SEQ ID NO:40.
Embodiment 74. An isolated monoclonal antibody comprising the heavy chain sequence of SEQ ID NO:42 and the light chain sequence of SEQ ID NO:44.
Embodiment 75. An isolated monoclonal antibody comprising the heavy chain sequence of SEQ ID NO:46 and the light chain sequence of SEQ ID NO:48.
Embodiment 76. An isolated monoclonal antibody comprising the heavy chain sequence of SEQ ID NO:50 and the light chain sequence of SEQ ID NO:52.
Embodiment 77. An isolated monoclonal antibody comprising the heavy chain sequence of SEQ ID NO:54 and the light chain sequence of SEQ ID NO:56.
Embodiment 78. An isolated monoclonal antibody comprising the heavy chain sequence of SEQ ID NO:58 and the light chain sequence of SEQ ID NO:60.
Embodiment 79. An isolated monoclonal antibody comprising the heavy chain sequence of SEQ ID NO:62 and the light chain sequence of SEQ ID NO:64.
Embodiment 80. The isolated monoclonal antibody or antigen-binding fragment thereof of any one of embodiments 1-79, wherein said monoclonal antibody or antigen-binding fragment thereof binds human Gal9 with a K_{d} of less than about 5 nM, 2 nM, 1 nM, 0.5 nM, 0.4 nM, 0.3 nM, 0.2 nM, 0.1 nM, 0.05 nM or 0.01 nM.
Embodiment 81. The isolated monoclonal antibody or antigen-binding fragment thereof of any one of embodiments 1-80, which binds to Gal9 and inhibits Gal9 binding to a Gal9 receptor (e.g., TIM3 or CD44).
Embodiment 82. The isolated monoclonal antibody or antigen-binding fragment thereof of any one of embodiments 1-80, which binds to Gal9 and does not inhibit Gal9 binding to a Gal9 receptor (e.g., TIM3 or CD44).
Embodiment 83. The isolated monoclonal antibody or antigen-binding fragment thereof of any one of embodiments 1-82, which reverses Gal9-induced Th1 apoptosis of T cells (such as CD4⁺ T cells).
Embodiment 84. The isolated monoclonal antibody or antigen-binding fragment thereof of any one of embodiments 1-83, which suppresses Gal9-induced Treg expansion.
Embodiment 85. A method of treating cancer in a subject in need thereof comprising administering to the subject an effective amount of the isolated monoclonal antibody or antigen-binding fragment thereof of any one of embodiments 1-84, thereby treating cancer in the subject.
Embodiment 86. A method of treating urothelial cancer, gastric cancer, gastroesophageal cancer, esophageal cancer, cervical cancer and/or head and neck squamous cancer in a subject in need thereof comprising administering to the subject an effective amount of the isolated monoclonal antibody or antigen-binding fragment thereof of any one of embodiments 1-84, thereby treating cancer in the subject.
Embodiment 87. A method of treating cancer in a subject in need thereof comprising administering to the subject an effective amount of the isolated monoclonal antibody or antigen-binding fragment thereof of any one of embodiments 1-84, and an anti-PD-1 antibody, thereby treating cancer in the subject.
Embodiment 88. A method of treating urothelial cancer, gastric cancer, gastroesophageal cancer, esophageal cancer, cervical cancer and/or head and neck squamous cancer in a subject in need thereof comprising administering to the subject an effective amount of the isolated monoclonal antibody or antigen-binding fragment thereof of any one of embodiments 1-84, an anti-PD-1 antibody and an anti-GITR antibody, thereby treating cancer in the subject.
Embodiment 89. A method of treating cancer in a subject in need thereof comprising administering to the subject an effective amount of the isolated monoclonal antibody or antigen-binding fragment thereof of any one of embodiments 1-84, and an anti-PD-1 antibody, thereby treating cancer in the subject.
Embodiment 90. A method of treating urothelial cancer, gastric cancer, gastroesophageal cancer, esophageal cancer, cervical cancer and/or head and neck squamous cancer in a subject in need thereof comprising administering to the subject an effective amount of the isolated monoclonal antibody or antigen-binding fragment thereof of any one of embodiments 1-69, an anti-PD-1 antibody and an anti-GITR antibody, thereby treating cancer in the subject.
Embodiment 91. The method of embodiment 85, 87 or 89, wherein the cancer is a hematological cancer or a solid tumor.
Embodiment 92. The method of embodiment 91, wherein the hematological cancer is selected from the group consisting of acute lymphoblastic leukemia (ALL), acute myeloid leukemia (AML), chronic lymphocytic leukemia, chronic myelogenous leukemia, and hairy cell leukemia, AIDS-related lymphoma, cutaneous T-cell lymphoma, Hodgkin lymphoma, Hodgkin lymphoma, mycosis fungoides, non-Hodgkin lymphoma, primary central nervous system lymphoma, Sézary syndrome, cutaneous T-Cell lymphoma, Waldenström macroglobulinemia, diffuse large B-Cell Lymphoma (DLBCL) and multiple myeloma.
Embodiment 93. The method of embodiment 91, wherein the solid tumor is breast cancer, head and neck cancer, lung cancer, melanoma, uveal melanoma, colorectal cancer, renal carcinoma, urothelial cancer, ovarian cancer, endometrial cancer, liver cancer, pancreatic cancer, cholangiocarcinoma, gastric cancer, gastroesophageal cancer, esophageal cancer, cervical cancer, head and neck squamous cancer or prostate cancer.
Embodiment 94. The method of any one of embodiments 85, 87, 89 or 91-93, further comprising administering to the patient a chemotherapeutic agent, an anti-angiogenesis agent, a growth inhibitory agent, an immune-oncology agent, a checkpoint inhibitor and/or an anti-neoplastic composition.
Embodiment 95. The method of embodiment 94, wherein the checkpoint inhibitor is an anti-PD-1.
Embodiment 96. The method of embodiment 94, wherein the immune-oncology agent is an anti-glucocorticoid-induced tumor necrosis factor receptor family-related protein (GITR) antibody.
Embodiment 97. A polynucleotide encoding the heavy chain or the light chain or the antigen-binding portion thereof of any one of embodiments 1-84.
Embodiment 98. A vector comprising the polynucleotide of embodiment 97, wherein the vector is an expression vector selected from the group consisting of a mammalian expression vector, a yeast expression vector, an insect expression vector, and a bacterial expression vector.
Embodiment 99. A method of rescuing or promoting effector T cell proliferation, enhancing effector T cell activity, and/or of identifying and treating cancer in a subject comprising:
(i) determining a level of Gal9 in a sample from the subject, and
(ii) administering to a subject having a level Gal9 higher than a reference level of Gal9 an effective amount of the isolated monoclonal antibody or antigen-binding fragment thereof of any one of embodiments 1-84,
thereby rescuing or promoting effector T cells proliferation, enhancing effector T cell activity, and/or of identifying and treating a subject.
Embodiment 100. A method of rescuing or promoting effector T cell proliferation, enhancing effector T cell activity, and/or of identifying and treating cancer in a subject comprising:
(i) determining a level of Gal9 in a sample from the subject, and
(ii) administering to a subject having a level Gal9 higher than a reference level of Gal9 an effective amount of the isolated monoclonal antibody or antigen-binding fragment thereof of any one of embodiments 1-84 and of an anti-PD-1 antibody,
thereby rescuing or promoting effector T cells proliferation, enhancing effector T cell activity, and/or of identifying and treating a subject.
Embodiment 101. A method of rescuing or promoting effector T cell proliferation, enhancing effector T cell activity, and/or of identifying and treating cancer in a subject comprising:
(i) determining a level of Gal9 in a sample from the subject, and
(ii) administering to a subject having a level Gal9 higher than a reference level of Gal9 an effective amount of the isolated monoclonal antibody or antigen-binding fragment thereof of any one of embodiments 1-84, and anti-PD-1 antibody and an anti-GITR antibody,
thereby rescuing or promoting effector T cells proliferation, enhancing effector T cell activity, and/or of identifying and treating a subject.
Embodiment 102. The method of any one of embodiments 99-101, wherein the subject is diagnosed with cancer, is at risk of developing cancer or has cancer relapse.
Embodiment 103. The method of one of embodiments 99-102, wherein the reference level of Gal9 is a level of Gal9 measured in a sample from a healthy or a control individual.
Embodiment 104. The method of any one of embodiments 99-103, wherein determining comprises comparing the level of Gal9 in the sample to the reference level.
Embodiment 105. The method of any one of embodiments 99-104, wherein the cancer is a hematological cancer or a solid tumor.
Embodiment 106. The method of embodiment 105, wherein the hematological cancer is selected from the group consisting of acute lymphoblastic leukemia (ALL), acute myeloid leukemia (AML), chronic lymphocytic leukemia, chronic myelogenous leukemia, and hairy cell leukemia, AIDS-related lymphoma, cutaneous T-cell lymphoma, Hodgkin lymphoma, Hodgkin lymphoma, mycosis fungoides, non-Hodgkin lymphoma, primary central nervous system lymphoma, Sézary syndrome, cutaneous T-Cell lymphoma, Waldenström macroglobulinemia, diffuse large B-Cell Lymphoma (DLBCL) and multiple myeloma.
Embodiment 107. The method of embodiment 105, wherein the solid tumor is breast cancer, head and neck cancer, lung cancer, melanoma, uveal melanoma, colorectal cancer, renal carcinoma, urothelial cancer, ovarian cancer, endometrial cancer, liver cancer, pancreatic cancer, cholangiocarcinoma, gastric cancer, gastroesophageal cancer, esophageal cancer, cervical cancer, head and neck squamous cancer or prostate cancer.
Embodiment 108. The method of any one of embodiments 99-107, wherein the sample is a blood sample, a plasma sample, a serum sample, a tumor biopsy sample, or a bone-marrow (BM)-derived mononuclear cell (MNC) sample.
Embodiment 109. The method of any one of embodiments 99 or 102-106, further comprising administering to the patient a chemotherapeutic agent, an anti-angiogenesis agent, a growth inhibitory agent, an immune-oncology agent, a checkpoint inhibitor and/or an anti-neoplastic composition.
Embodiment 110. The method of embodiment 109, wherein the checkpoint inhibitor is an anti-PD-1.
Embodiment 111. The method of claim 110, wherein the immune-oncology agent is an anti-glucocorticoid-induced tumor necrosis factor receptor family-related protein (GITR) antibody.

### EXAMPLES

### Example 1

### Humanized Anti-Gal9 Antibodies Affinity Maturation

In order to develop anti-Gal9 antibodies with improved binding affinity to human Gal9, as compared to the original Ab A anti-Gal9 antibody, random mutations in 3 CDRs: H1-H2-L2 or L1-L3-H3 were performed, and libraries of variants were generated.

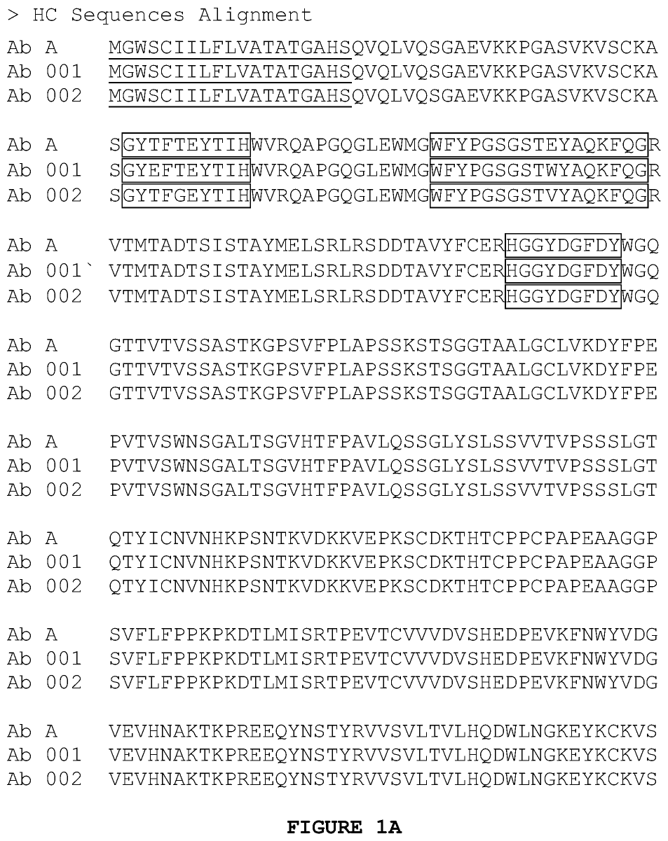

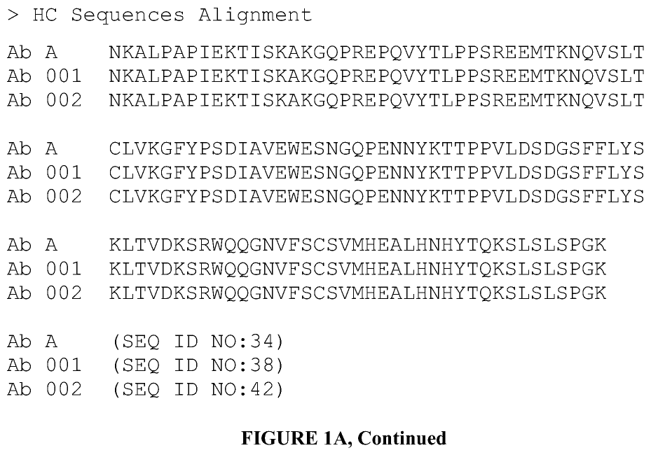

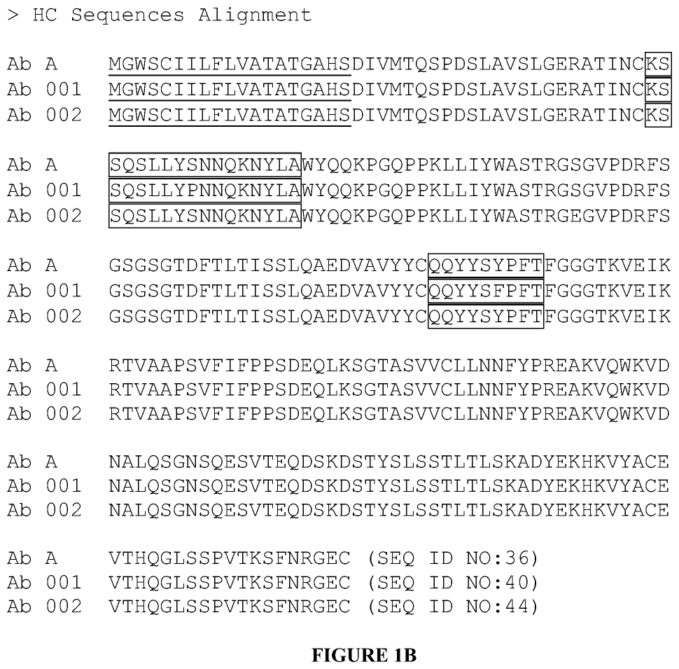

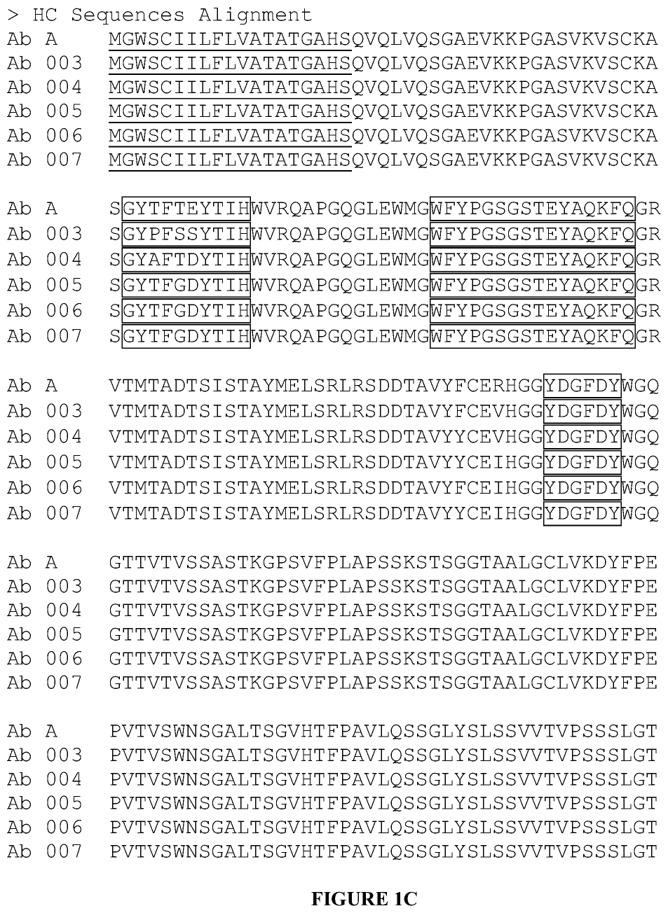

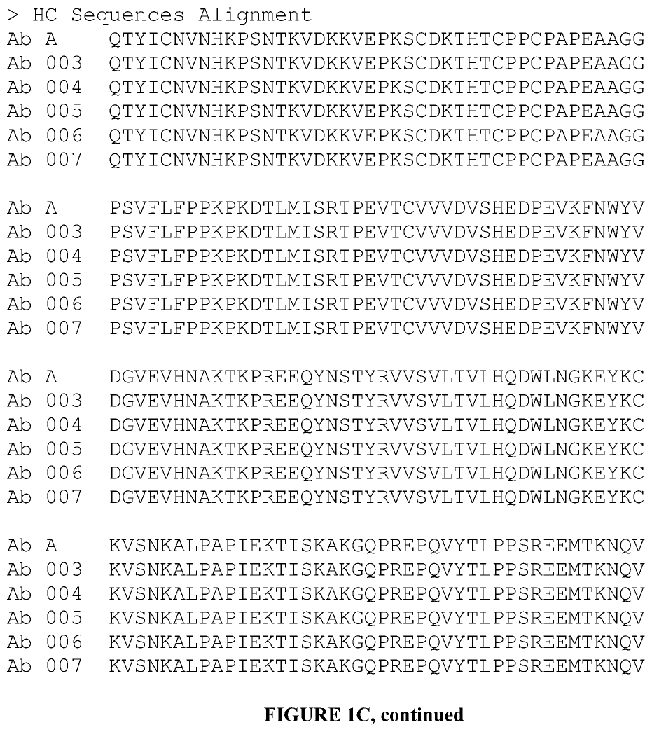

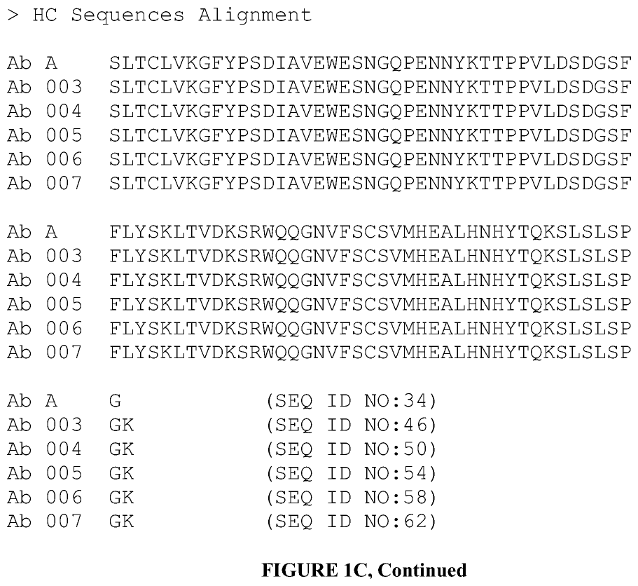

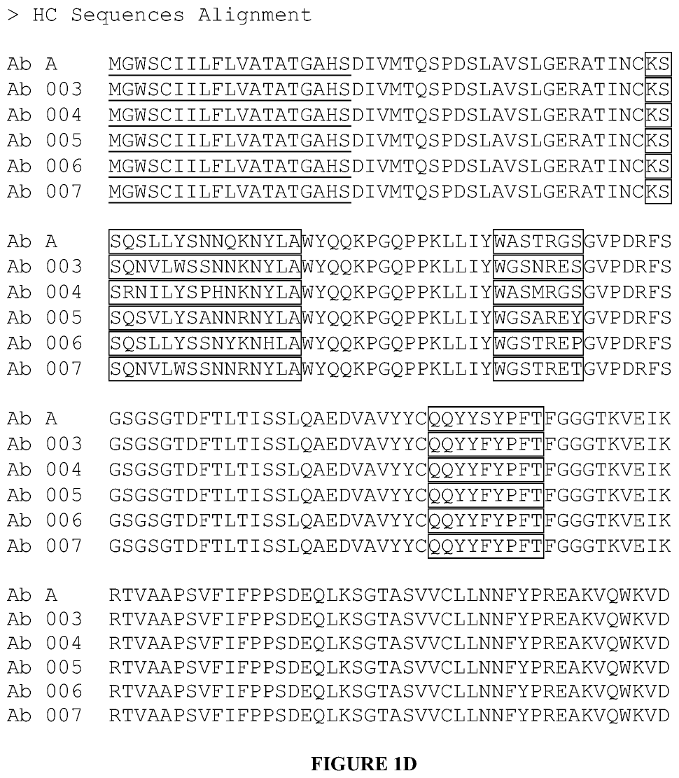

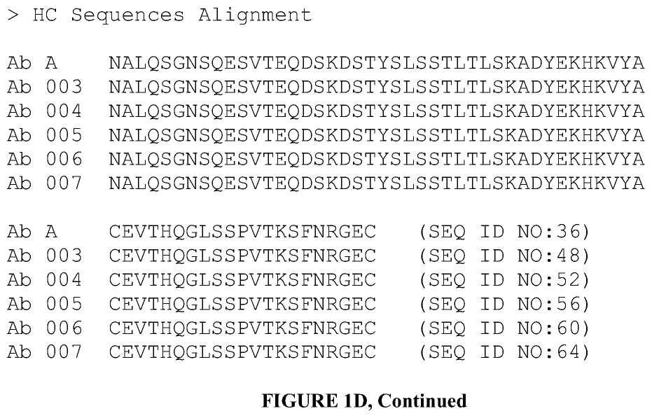

Two antibodies were identified as having the best binding affinity as compared to the Ab A anti-Gal9 antibody: anti-gal9 antibodies Ab 001 and Ab 002. The alignments of the sequences of the heavy chain and light chain are illustrated FIGS. 1A and 1B, respectively, where the CDRs are squared in the sequences.

Affinity binding of the antibodies were measured using surface plasmon resonance and solution equilibrium assay. As shown in Table 2, anti-Gal9 antibodies Ab 001 and Ab 002 were found to have a greater binding affinity than the Ab A antibody, as defined by the reduction in K_{D} values.

TABLE 2
Binding data of affinity matured antibodies

| Sequence | K_{D} (pM) | kₐ | k_{d} | K_{D} (pM) SEA | |
|---|---|---|---|---|---|
| ID | SPR assay | | | human | mouse |
| Ab A | 7179 | 1.4E+06 | 9.9E−03 | 3100 | 49,000 |
| Ab 001 | 211 | 2.4E+06 | 4.7E−03 | 350 | >60,000 |
| Ab 002 | 563 | 8.3E+06 | 5.2E−04 | 1500 | 29,000 |

Separately, in order to develop anti-Gal9 antibodies with improved binding affinity to human Gal9, as compared to the original Ab A anti-Gal9 antibody, a yeast display approach was used.

Five antibodies were identified as having the best binding affinity as compared to the Ab A anti-Gal9 antibody: anti-gal9 antibodies Ab 003, Ab 004, Ab 005, Ab 006 and Ab 007. The alignments of the sequences of the heavy chain and light chain are illustrated in FIGS. 1C and 1D, respectively, where the CDRs are squared in the sequences.

Affinity binding of the antibodies were measured using surface plasmon resonance and solution equilibrium assay. As shown in Table 3, anti-Gal9 antibodies Ab 003-007 were found to have a greater binding affinity than antibody Ab A, as defined by the reduction in K_{D} values. Surprisingly at least antibodies Ab 003-005 and Ab 007 also showed substantially improved affinity to mouse Gal9 relative to antibody Ab A.

TABLE 3
Binding data of affinity maturated antibodies

| Sequence | K_{D} (pM) | kₐ | k_{d} | K_{D} (pM) SEA | |
|---|---|---|---|---|---|
| ID | SPR assay | | | human | mouse |
| Ab A | 4900 | 1.6E+05 | 7.7E−04 | 3100 | 49,000 |
| Ab 003 | 110 | 9.3E+04 | 1.0E−05 | 650 | 1240 |
| Ab 004 | 450 | 9.6E+04 | 4.3E−05 | 740 | 4470 |
| Ab 005 | 510 | 9.3E+04 | 5.4E−05 | 800 | 1490 |
| Ab 006 | 620 | 1.0E+05 | 6.6E−05 | no signal | no signal |
| Ab 007 | 1400 | 7.7E+04 | 1.1E−04 | 1010 | 1760 |

### Example 2

### Effect of Anti-Gal9 Antibodies on Gal9-Induced Apoptosis

In order to characterize the affinity matured Ab 001 anti-Gal9 antibody, as compared to the parental Ab A anti-Gal9 antibody, a comparison of the effect of the antibodies on Gal9-induced apoptosis was performed.

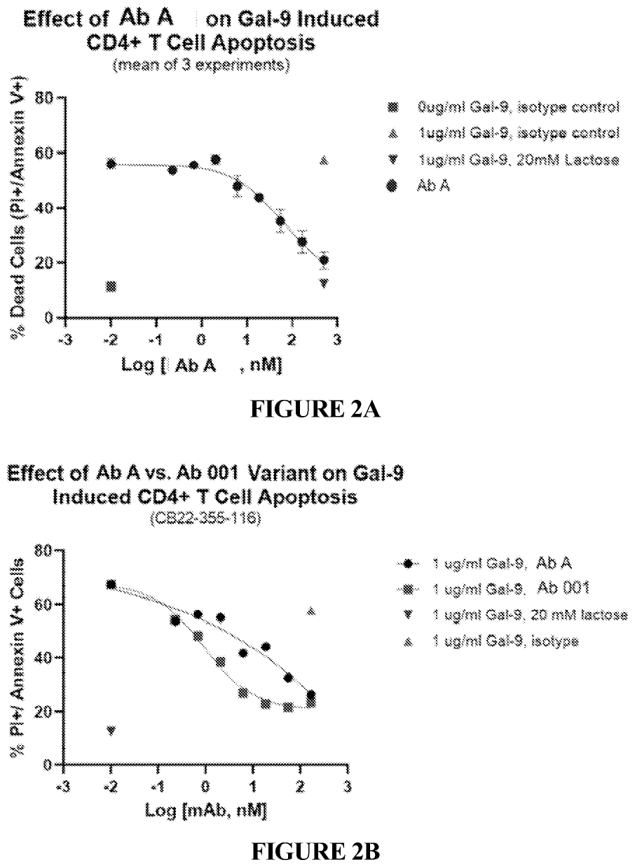

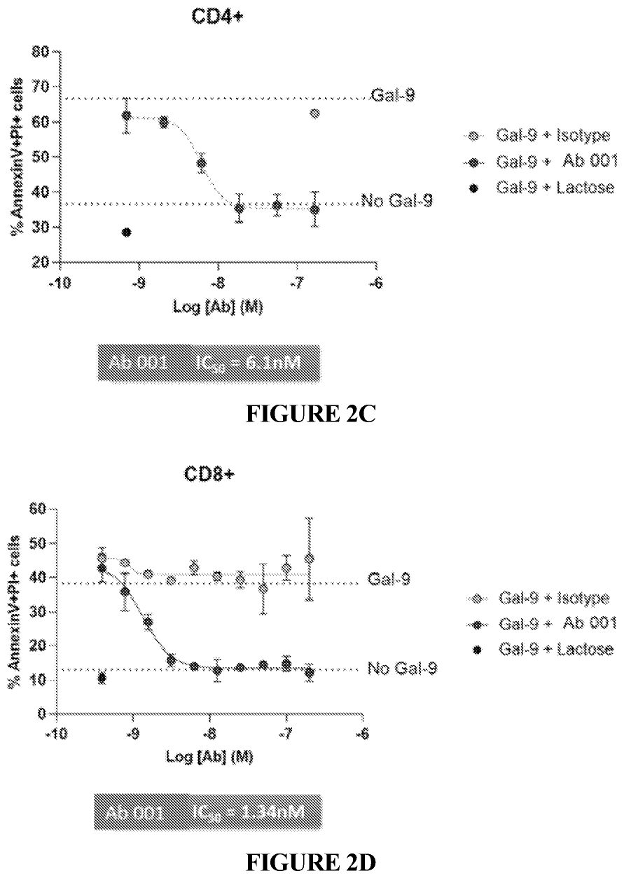

As illustrated in FIG. 2B, the affinity matured anti-Gal9 antibody was found to be more potent at reducing CD4+ T cell apoptosis as compared to the Ab A antibody (also shown in FIG. 2A). The calculated IC50 values were 18.7 nM for Ab A and 1.9 nM for Ab 001.

The T-cell apoptosis assay will be used to evaluate the efficacy of the Ab 002 antibody to inhibit Gal9-induced apoptosis.

Additional characterization of the antibodies to be performed will include evaluation of the cross-reactivity of the antibodies to other Gal9 family members, biochemical and biophysical properties assessment (including aggregation data for example), deamination, oxidation evaluation, proteolysis (specifically before and after thermal stress).

The ability of anti-Gal9 antibody Ab 001 to inhibit Gal9 induced CD4⁺ and/or CD8⁺ T-cell apoptosis was assessed by flow cytometric analysis of annexin V⁺ and propidium iodide⁺ (PI) double positive cells.

Either CD4⁺ or CD8⁺ T-cells were activated with the mitogenic stimuli anti-CD3 and anti-CD28, along with Gal9. Activated T-cells were either co-cultured with anti-Gal9 antibody Ab 001 or isotype control. As illustrated in FIGS. 2C and 2D, inclusion of Ab 001, as compared to isotype control, resulted in a significant diminution of both CD4⁺ and CD8⁺ T-cell apoptosis.

### Example 3

### Evaluation of Ex-Vivo AML Apoptosis

Effects of anti-Gal9 affinity matured antibodies (Ab 001 and Ab 002) will be evaluated on leukemia stem cells (LSC, CD34+, CD38−) apoptosis, and compared to the effects induced by the non-affinity matured anti-Gal9 antibody (Ab A).

AML bone marrow samples will be collected and frozen until used for the experiments. LSC cells will be sorted from the samples based on their expression of CD34 and CD38.

Tim3/Gal9 expression levels will be assessed at the cell surface, as well as the levels of secreted/released gal9. Cells will be cultured in suspension for 96 h hours, in the presence of or the absence of the antibody, and apoptosis will be assessed by flow cytometry. Internal controls will include other existing anti-Gal9 antibodies, a positive control (aGal9), and a negative control.

### Example 4

### Evaluation of the Effect of the Affinity Matured Anti-Gal9 Antibodies on AML Patient Samples In-Vivo

In order to assess the efficacy of the affinity matured anti-Gal9 antibodies in vivo, NOD scid gamma (NSG) immunocompromised mice will be engrafted using AML in vivo systemic models. FLT3, IDH1/2, and TP53 genes will be sequenced for mutants' identification; cytogenetics, clinical, immunophenotyping assessment will be performed.

Systemic engraftment kinetics will be measured.

The engraftment period will last for about 6-12 weeks and be followed by the assessment of AML engraftment by flow cytometry to detect CD45/CD33 cells. AML agents will then be tested. The endpoint of the experiments will be survival, which is when flow cytometry analysis of splenocytes, whole blood, bone marrow CD45/CD33 will be performed, with the animals being treated with a single anti-Gal9 antibody or a combination of an anti-Gal9 antibody with chemotherapy.

As an alternative to the humanized AML model an autologous AML platform will be used, to provide the ability to test immuno-oncology therapeutics in an autologous co-culture system (as single agents or in combination), with access to Champions' well-characterized bank of AML patient models. Patients' primary AML cells will be co-cultured with patients' matched T cells, in the presence of in the absence of the antibody of interest (alone or in combination with another therapeutic agent). The cells will be grown in an enriched growth media, and various parameters will be evaluated, including phenotyping by flow cytometry, T cell activation, cytokine production by Luminex, and AML frequency.

As a proof-of-concept experiment, a model of solid tumors using MC38 mouse syngeneic model (colorectal cancer), will be used for further in vivo analysis. Single therapy study using an anti-Gal9 antibody, or an anti-PD-1 antibody will be performed in the MC38 animals, as well as a dose response experiment. Combination study of the anti-Gal9 antibody and an anti-PD-1 will be performed to understand the mechanism of action in vivo.

### Example 5

### Evaluation of Ab 001 Inhibitory Effect of Gal9-Induced Tim-3 Dimerization

TIM-3 dimerization was assessed by Eurofins DiscoverX assay. The assay relies on enzyme fragment complementation in which the enzyme β-galactosidase is split into two fragments, an enzyme acceptor (EA) and an enzyme donor (ED), fused to the cytoplasmic tails of the receptor TIM-3. When the ligand Gal9 binds its receptor, TIM-3, the receptor homodimerizes, bringing together EA and ED which metabolize the assay substrate and generate quantifiable light photons.

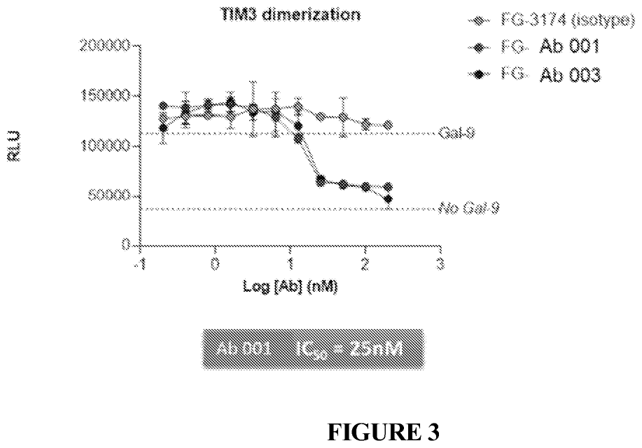

As illustrated in FIG. 3, inclusion of anti-Gal9 antibodies Ab001 or Ab003, compared to isotype control, inhibited the ability of soluble Gal9 from forming TIM-3 homodimers as evidenced by quenching of emitted photons.

### Example 6

### Efficacy of Anti-Gal9 Antibody as a Combination Therapy in A Proof-of-Concept Mouse Model

Anti-Gal9 antibody was tested individually or in combination with checkpoint inhibitor antibodies to assess efficacy of inhibiting tumor growth in the MC38 mouse syngeneic colon carcinoma model. Antibodies tested included anti-GITR, anti-PD-1 and anti-Gal9. Two anti-Gal9 antibodies were used in separate experiments, a commercially available anti-Gal9 antibody (antiGal9 Ab RG9-1) and one of the antibodies of the invention (Ab 003).

While all monotherapies appeared to provide minimal tumor growth inhibition compared to isotype control in this model, combination therapy with anti-Gal9 antibody and either anti-GITR or anti-PD-1 resulted in modest tumor growth inhibition (49-51% tumor growth inhibition for Ab 003 in a double therapy). Triple therapy with anti-Gal9, anti-GITR, and anti-PD1 provided even greater tumor growth inhibition (67% tumor growth inhibition for Ab 003 in a triple therapy).

### Example 7

### Clinical Synopsis of Phase 1a/1b in Relapsed/Refractory Advanced Select Solid Tumors

A phase 1a/1b clinical trial will evaluate the maximum tolerated dose (MTD) of an anti-Gal9 antibody of the invention in patients with relapsed/refractory advanced solid tumors, following the BOIN design method.

The indications are selected based on Gal9 expression levels, and are as follows: colorectal carcinoma, gastric cancer, gastroesophageal junction adenocarcinoma, esophageal cancer, urothelial carcinoma, cervical carcinoma, and head and neck squamous cell carcinoma. The dose escalation phase is a standard 3+3 Bayesian optimal interval design. Once a recommended dose is established, the dose expansion phase includes administering two dose levels of anti-Gal9, plus or minus pembrolizumab, until a minimum safe biologically effective dose is confirmed. A Phase II trial is planned after the completion of the dose expansion phase.

Although the invention has been described with reference to the above examples, it will be understood that modifications and variations are encompassed within the spirit and scope of the invention. Accordingly, the invention is limited only by the following claims.

Sequences:
Underline: signal sequence

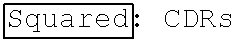

>VH-CDR1
(SEQ ID NO: 1)
GYX₁FX₂X₃YTIH
X₁ is T, E, P, or A
X₂ is T, G, or S
X₃ is E, S, or D
>VH-CDR2
(SEQ ID NO: 8)
WFYPGSGSTX₄YAQKFQG
X₄ is E, W, or V
>VH-CDR3
(SEQ ID NO: 12)
HGGYDGFDY
>VL-CDR1
(SEQ ID NO: 13)
KSSX₅X₆X₇LX₈X₉X₁₀X₁₁X₁₂X₁₃NX₁₄LA
X₅ is Q or R
X₆ is S or N
X₇ is L, V, or I
X₈ is Y or W
X₉ is S or P
X₁₀ is N, S, P, or A
X₁₁ is N or H
X₁₂ is Q, N, or Y
X₁₃ is K or R
X₁₄ is Y or H
>VL-CDR2
WX₁₅SX₁₆RX₁₇X₁₈
X₁₅ is A or G
X₁₆ is T, N, M, or A
X₁₇ is G or E
X₁₈ is S, E, Y, P, or T
>VL-CDR3
(SEQ ID NO: 30)
QQYYX₁₉X₂₀PFT
X₁₉ is S or F
X₂₀ is Y or F
>Ab A HC
(SEQ ID NO: 34)

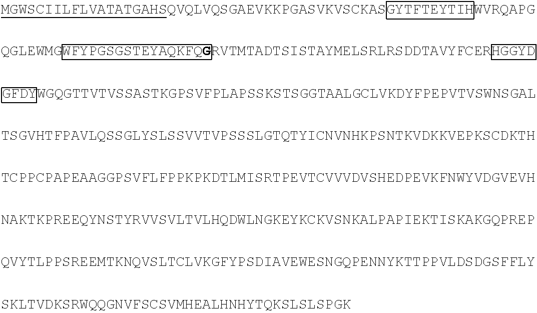

>Ab A VH
(SEQ ID NO: 35)

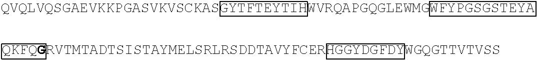

>Ab A VH-CDR1
(SEQ ID NO: 2)
GYTFTEYTIH
>Ab A VH-CDR2
(SEQ ID NO: 9)
WFYPGSGSTEYAQKFQG
>Ab A VH-CDR3
(SEQ ID NO: 12)
HGGYDGFDY
>Ab A HFR1
(SEQ ID NO: 66)
QVQLVQSGAEVKKPGASVKVSCKAS
>Ab A HFR2
(SEQ ID NO: 67)
WVRQAPGQGLEWMG
>Ab A HER3
(SEQ ID NO: 68)
RVTMTADTSISTAYMELSRLRSDDTAVYFCER
>Ab A HFR4
(SEQ ID NO: 71)
WGQGTTVTVSS
>Ab A LC
(SEQ ID NO: 36)

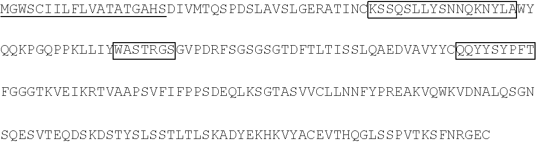

>Ab A VL
(SEQ ID NO: 37)

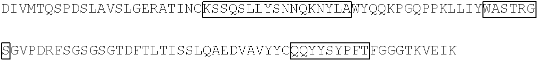

>Ab A VL-CDR1
(SEQ ID NO: 14)
KSSQSLLYSNNQKNYLA
>Ab A VL-CDR2
(SEQ ID NO: 22)
WASTRGS
>Ab A VL-CDR3
(SEQ ID NO: 31)
QQYYSYPFT
>Ab A LFR1
(SEQ ID NO: 72)
DIVMTQSPDSLAVSLGERATINC
>Ab A LFR2
(SEQ ID NO: 73)
WYQQKPGQPPKLLIY
>Ab A LER3
(SEQ ID NO: 74)
GVPDRFSGSGSGTDETLTISSLQAEDVAVYYC
>Ab A LER4
(SEQ ID NO: 75)
FGGGTKVEIK
>Ab 001 HC
(SEQ ID NO: 38)

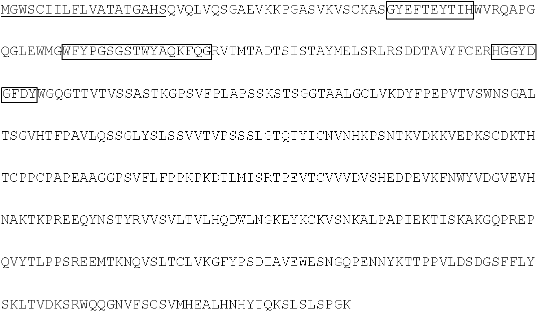

>Ab 001 VH
(SEQ ID NO: 39)

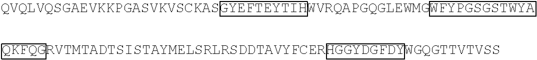

>Ab 001 VH-CDR1
(SEQ ID NO: 3)
GYEFTEYTIH
>Ab 001 VH-CDR2
(SEQ ID NO: 10)
WFYPGSGSTWYAQKFQG
>Ab 001 VH-CDR3
(SEQ ID NO: 12)
HGGYDGFDY
>Ab 001 HFR1
(SEQ ID NO: 66)
QVQLVQSGAEVKKPGASVKVSCKAS
>Ab 001 HFR2
(SEQ ID NO: 67)
WVRQAPGQGLEWMG
>Ab 001 HFR3
(SEQ ID NO: 68)
RVTMTADTSISTAYMELSRLRSDDTAVYFCER
>Ab 001 HFR4
(SEQ ID NO: 71)
WGQGTTVTVSS
>Ab 001 LC
(SEQ ID NO: 40)

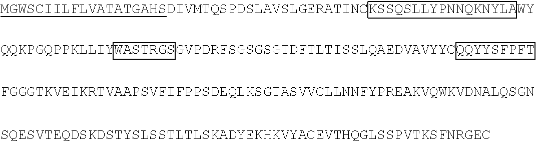

>Ab 001 VL
(SEQ ID NO: 41)

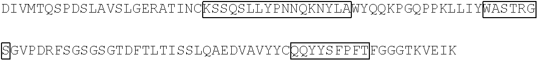

>Ab 001 VL-CDR1
(SEQ ID NO: 15)
KSSQSLLYPNNQKNYLA
>Ab 001 VL-CDR2
(SEQ ID NO: 23)
WASTRGS
>Ab 001 VL-CDR3
(SEQ ID NO: 32)
QQYYSFPFT
>Ab 001 LFR1
(SEQ ID NO: 72)
DIVMTQSPDSLAVSLGERATINC
>Ab 001 LFR2
(SEQ ID NO: 73)
WYQQKPGQPPKLLIY
>Ab 001 LFR3
(SEQ ID NO: 74)
GVPDRFSGSGSGTDFTLTISSLQAEDVAVYYC
>Ab 001 LFR4
(SEQ ID NO: 75)
FGGGTKVEIK
>Ab 002 HC
(SEQ ID NO: 42)

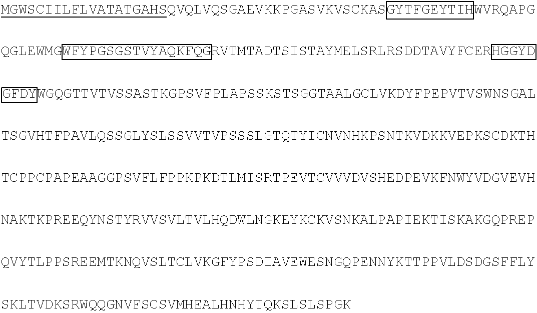

>Ab 002 VH
(SEQ ID NO: 43)

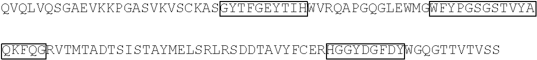

>Ab 002 VH-CDR1
(SEQ ID NO: 4)
GYTFGEYTIH
>Ab 002 VH-CDR2
(SEQ ID NO: 11)
WFYPGSGSTVYAQKFQG
>Ab 002 VH-CDR3
(SEQ ID NO: 12)
HGGYDGFDY
>Ab 002 HFR1
(SEQ ID NO: 66)
QVQLVQSGAEVKKPGASVKVSCKAS
>Ab 002 HFR2
(SEQ ID NO: 67)
WVRQAPGQGLEWMG
>Ab 002 HER3
(SEQ ID NO: 68)
RVTMTADTSISTAYMELSRLRSDDTAVYFCER
>Ab 002 HFR4
(SEQ ID NO: 71)
WGQGTTVTVSS
>Ab 002 (E07) LC
(SEQ ID NO: 44)

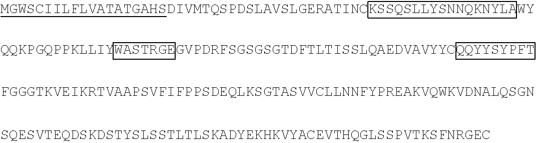

>Ab 002 VL
(SEQ ID NO: 45)

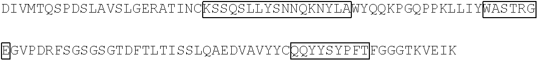

>Ab 002 VL-CDR1
(SEQ ID NO: 14)
KSSQSLLYSNNQKNYLA
>Ab 002 VL-CDR2
(SEQ ID NO: 24)
WASTRGE
>Ab 002 VL-CDR3
(SEQ ID NO: 31)
QQYYSYPFT
>Ab 002 LFR1
(SEQ ID NO: 72)
DIVMTQSPDSLAVSLGERATINC
>Ab 002 LFR2
(SEQ ID NO: 73)
WYQQKPGQPPKLLIY
>Ab 002 LFR3
(SEQ ID NO: 74)
GVPDRESGSGSGTDFTLTISSLQAEDVAVYYC
>Ab 002 LFR4
(SEQ ID NO: 75)
FGGGTKVEIK
>Ab 003 HC
(SEQ ID NO: 46)

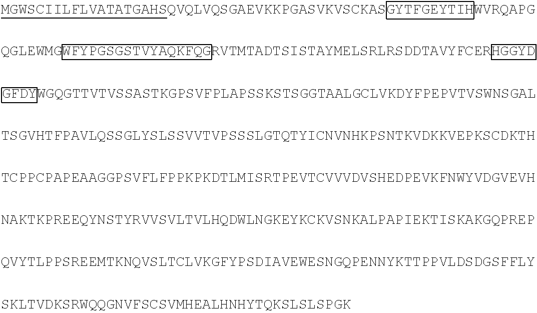

>Ab 003 VH
(SEQ ID NO: 47)

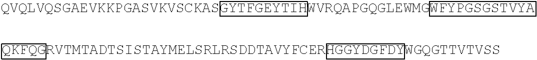

>Ab 003 VH-CDR1
(SEQ ID NO: 5)
GYPFSSYTIH
>Ab 003 VH-CDR2
(SEQ ID NO: 9)
WFYPGSGSTEYAQKFQG
>Ab 003 VH-CDR3
(SEQ ID NO: 12)
HGGYDGFDY
>Ab 003 HFR1
(SEQ ID NO: 66)
QVQLVQSGAEVKKPGASVKVSCKAS
>Ab 003 HFR2
(SEQ ID NO: 67)
WVRQAPGQGLEWMG
>Ab 003 HFR3
(SEQ ID NO: 69)
RVTMTADTSISTAYMELSRLRSDDTAVYFCEV
>Ab 003 HFR4
(SEQ ID NO: 71)
WGQGTTVTVSS
>Ab 003 LC
(SEQ ID NO: 48)

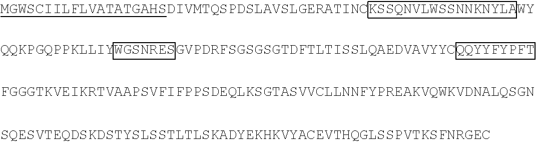

>Ab 003 VL
(SEQ ID NO: 49)

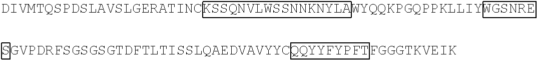

>Ab 003 VL-CDR1
(SEQ ID NO: 16)
KSSQNVLWSSNNKNYLA
>Ab 003 VL-CDR2
(SEQ ID NO: 25)
WGSNRES
>Ab 003 VL-CDR3
(SEQ ID NO: 33)
QQYYFYPFT
>Ab 003 LFR1
(SEQ ID NO: 72)
DIVMTQSPDSLAVSLGERATINC
>Ab 003 LFR2
(SEQ ID NO: 73)
WYQQKPGQPPKLLIY
>Ab 003 LFR3
(SEQ ID NO: 74)
GVPDRESGSGSGTDFTLTISSLQAEDVAVYYC
>Ab 003 LFR4
(SEQ ID NO: 75)
FGGGTKVEIK
>Ab 004 HC
(SEQ ID NO: 50)

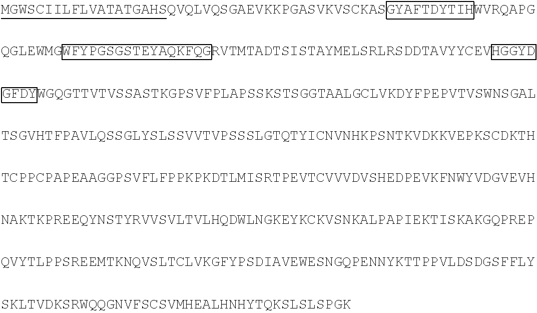

>Ab 004 VH
(SEQ ID NO: 51)

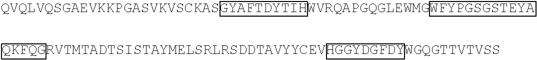

>Ab 004 VH-CDR1
(SEQ ID NO: 6)
GYAFTDYTIH
>Ab 004 VH-CDR2
(SEQ ID NO: 9)
WFYPGSGSTEYAQKFQG
>Ab 004 VH-CDR3
(SEQ ID NO: 12)
HGGYDGFDY
>Ab 004 HFR1
(SEQ ID NO: 66)
QVQLVQSGAEVKKPGASVKVSCKAS
>Ab 004 HFR2
(SEQ ID NO: 67)
WVRQAPGQGLEWMG
>Ab 004 HFR3
(SEQ ID NO: 76)
RVTMTADTSISTAYMELSRLRSDDTAVYYCEV
>Ab 004 HFR4
(SEQ ID NO: 71)
WGQGTTVTVSS
>Ab 004 LC
(SEQ ID NO: 52)

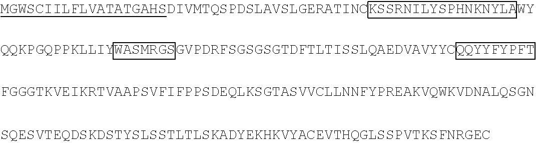

>Ab 004 VL
(SEQ ID NO: 53)

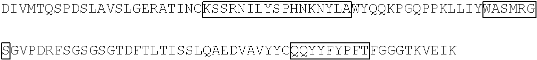

>Ab 004 VL-CDR1
(SEQ ID NO: 17)
KSSRNILYSPHNKNYLA
>Ab 004 VL-CDR2
(SEQ ID NO: 26)
WASMRGS
>Ab 004 VL-CDR3
(SEQ ID NO: 33)
QQYYFYPFT
>Ab 004 LFR1
(SEQ ID NO: 72)
DIVMTQSPDSLAVSLGERATINC
>Ab 004 LFR2
(SEQ ID NO: 73)
WYQQKPGQPPKLLIY
>Ab 004 LFR3
(SEQ ID NO: 74)
GVPDRESGSGSGTDFTLTISSLQAEDVAVYYC
>Ab 004 LFR4
(SEQ ID NO: 75)
FGGGTKVEIK
>Ab 005 HC
(SEQ ID NO: 54)

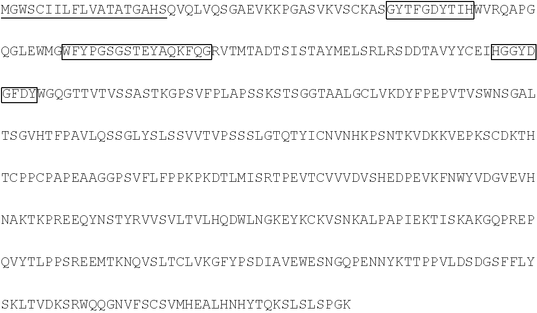

>Ab 005 VH
(SEQ ID NO: 55)

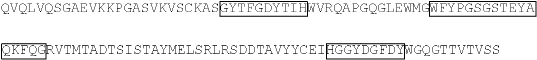

>Ab 005 VH-CDR1
(SEQ ID NO: 7)
GYTFGDYTIH
>Ab 005 VH-CDR2
(SEQ ID NO: 9)
WFYPGSGSTEYAQKFQG
>Ab 005 VH-CDR3
(SEQ ID NO: 12)
HGGYDGFDY
>Ab 005 HFR1
(SEQ ID NO: 66)
QVQLVQSGAEVKKPGASVKVSCKAS
>Ab 005 HFR2
(SEQ ID NO: 67)
WVRQAPGQGLEWMG
>Ab 005 HFR3
(SEQ ID NO: 70)
RVTMTADTSISTAYMELSRLRSDDTAVYYCEI
>Ab 005 HFR4
(SEQ ID NO: 71)
WGQGTTVTVSS
>Ab 005 LC
(SEQ ID NO: 56)

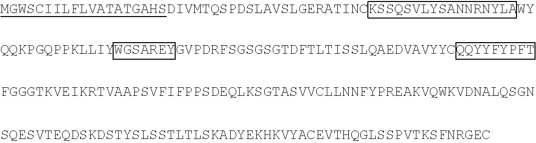

>Ab 005 VL
(SEQ ID NO: 57)

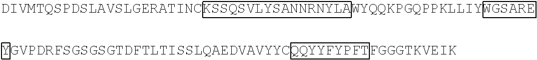

>Ab 005 VL-CDR1
(SEQ ID NO: 18)
KSSQSVLYSANNRNYLA
>Ab 005 VL-CDR2
(SEQ ID NO: 27)
WGSAREY
>Ab 005 VL-CDR3
(SEQ ID NO: 33)
QQYYFYPFT
>Ab 005 LFR1
(SEQ ID NO: 72)
DIVMTQSPDSLAVSLGERATINC
>Ab 005 LFR2
(SEQ ID NO: 73)
WYQQKPGQPPKLLIY
>Ab 005 LFR3
(SEQ ID NO: 74)
GVPDRFSGSGSGTDFTLTISSLQAEDVAVYYC
>Ab 005 LFR4
(SEQ ID NO: 75)
FGGGTKVEIK
>Ab 006 HC
(SEQ ID NO: 58)

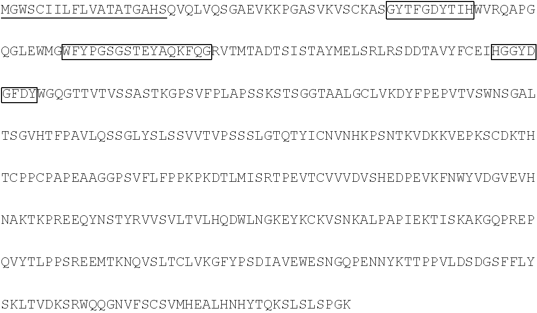

>Ab 006 VH
(SEQ ID NO: 59)

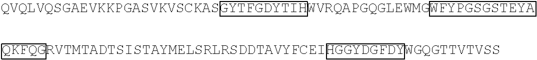

>Ab 006 VH-CDR1
(SEQ ID NO: 7)
GYTFGDYTIH
>Ab 006 VH-CDR2
(SEQ ID NO: 9)
WFYPGSGSTEYAQKFQG
>Ab 006 VH-CDR3
(SEQ ID NO: 12)
HGGYDGFDY
>Ab 006 HFR1
(SEQ ID NO: 66)
QVQLVQSGAEVKKPGASVKVSCKAS
>Ab 006 HFR2
(SEQ ID NO: 67)
WVRQAPGQGLEWMG
>Ab 006 HFR3
(SEQ ID NO: 77)
RVTMTADTSISTAYMELSRLRSDDTAVYFCEI
>Ab 006 HFR4
(SEQ ID NO: 71)
WGQGTTVTVSS
>Ab 006 LC
(SEQ ID NO: 60)

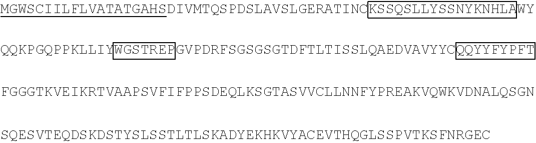

>Ab 006 VL
(SEQ ID NO: 61)

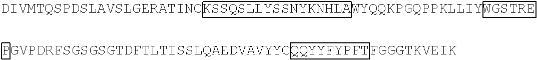

>Ab 006 VL-CDR1
(SEQ ID NO: 19)
KSSQSLLYSSNYKNHLA
>Ab 006 VL-CDR2
(SEQ ID NO: 28)
WGSTREP
>Ab 006 VL-CDR3
(SEQ ID NO: 33)
QQYYFYPFT
>Ab 006 LER1
(SEQ ID NO: 72)
DIVMTQSPDSLAVSLGERATINC
>Ab 006 LFR2
(SEQ ID NO: 73)
WYQQKPGQPPKLLIY
>Ab 006 LFR3
(SEQ ID NO: 74)
GVPDRESGSGSGTDFTLTISSLQAEDVAVYYC
>Ab 006 LER4
(SEQ ID NO: 75)
FGGGTKVEIK
>Ab 007 HC
(SEQ ID NO: 62)

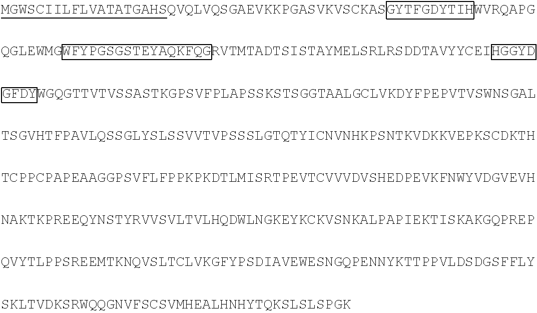

>Ab 007 VH
(SEQ ID NO: 63)

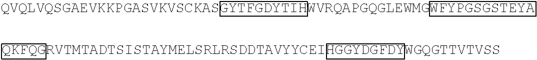

>Ab 007 VH-CDR1
(SEQ ID NO: 7)
GYTFGDYTIH
>Ab 007 VH-CDR2
(SEQ ID NO: 9)
WFYPGSGSTEYAQKFQG
>Ab 007 VH-CDR3
(SEQ ID NO: 12)
HGGYDGFDY
>Ab 007 HER1
(SEQ ID NO: 66)
QVQLVQSGAEVKKPGASVKVSCKAS
>Ab 007 HER2
(SEQ ID NO: 67)
WVRQAPGQGLEWMG
>Ab 007 HFR3
(SEQ ID NO: 70)
RVTMTADTSISTAYMELSRLRSDDTAVYYCEI
>Ab 007 HFR4
(SEQ ID NO: 71)
WGQGTTVTVSS
>Ab 007 LC
(SEQ ID NO: 64)

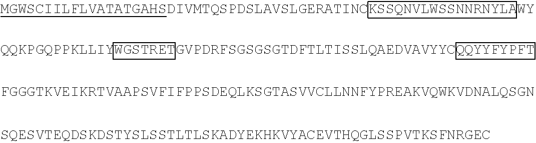

>Ab 007 VL
(SEQ ID NO: 65)

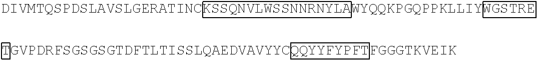

>Ab 007 VL-CDR1
(SEQ ID NO: 20)
KSSQNVLWSSNNRNYLA
>Ab 007 VL-CDR2
(SEQ ID NO: 29)
WGSTRET
>Ab 007 VL-CDR3
(SEQ ID NO: 33)
QQYYFYPFT
>Ab 007 LER1
(SEQ ID NO: 72)
DIVMTQSPDSLAVSLGERATINC
>Ab 007 LFR2
(SEQ ID NO: 73)
WYQQKPGQPPKLLIY
>Ab 007 LFR3
(SEQ ID NO: 74)
GVPDRFSGSGSGTDFTLTISSLQAEDVAVYYC
>Ab 007 LFR4
(SEQ ID NO: 75)
FGGGTKVEIK

1. An isolated monoclonal antibody or an antigen-binding fragment thereof comprising:
(a) a heavy chain variable region (HCVR) comprising
(i) a HCVR CDR1 sequence having the amino acid sequence GYX₁FX₂X₃YTIH (SEQ ID NO:1), wherein X₁ is selected from T, E, P, or A; X₂ is selected from T, G, or S; and X₃ is selected from E, S, or D;
(ii) a HCVR CDR2 sequence having the amino acid sequence WFYPGSGSTX₄YAQKFQG (SEQ ID NO:8), wherein X₄ is selected from E, W, or V; and
(iii) a HCVR CDR3 sequence having the amino acid sequence HGGYDGFDY (SEQ ID NO:12); and
(b) a light chain variable region (LCVR) comprising
(i) a LCVR CDR1 sequence having the amino acid sequence KSSX₅X₆X₇LX₈X₉X₁₀X₁₁X₁₂X₁₃NX₁₄LA (SEQ ID NO:13), wherein X₅ is selected from Q or R; X₆ is selected from S or N; X₇ is selected from L, V, or I; X₈ is selected from Y or W; X₉ is selected from S or P; X₁₀ is selected from N, S, P, or A; X₁₁ is selected from N or H; X₁₂ is selected from Q, N, or Y; X₁₃ is selected from K or R; and X₁₄ is selected from Y or H;
(ii) a LCVR CDR2 sequence having the amino acid sequence WX₁₅SX₁₆RX₁₇X₁₈, wherein X₁₅ is selected from A or G; X₁₆ is selected from T, N, M, or A; X₁₇ is selected from G or E; and X₁₈ is selected from S, E, Y, P, or T; and
(iii) a LCVR CDR3 sequence having the amino acid sequence QQYYX₁₉X₂₀PFT (SEQ ID NO:30), wherein X₁₉ is selected from S or F; and X₂₀ is selected from Y or F,
wherein the antibody or antigen-binding fragment thereof specifically binds Galectin-9 (Gal9), with the proviso that the antibody does not comprise HCVR CDR1 sequence GYTFTEYTIH (SEQ ID NO:2), HCVR CDR2 sequence WFYPGSGSTEYAQKFQG (SEQ ID NO:9), HCVR CDR3 sequence HGGYDGFDY (SEQ ID NO:12), LCVR CDR1 sequence KSSQSLLYSNNQKNYLA (SEQ ID NO:14), LCVR CDR2 sequence WASTRGS (SEQ ID NO:22), and LCVR CDR3 sequence QQYYSYPFT (SEQ ID NO:31).

2. The isolated monoclonal antibody or an antigen-binding fragment thereof of claim 1 comprising:
(a) a HCVR comprising
(i) a HCVR CDR1 sequence having the amino acid sequence GYX₁FX₂X₃YTIH (SEQ ID NO:1), wherein X₁ is selected from T or E, and X₂ is selected from T or G, and X₃ is E;
(ii) a HCVR CDR2 sequence having the amino acid sequence WFYPGSGSTX₄YAQKFQG (SEQ ID NO:8), wherein X₄ is selected from W or V; and
(iii) a HCVR CDR3 sequence having the amino acid sequence HGGYDGFDY (SEQ ID NO:12); and
(b) a LCVR comprising
(i) a LCVR CDR1 sequence having the amino acid sequence KSSX₅X₆X₇LX₈X₉X₁₀X₁₁X₁₂X₁₃N X₁₄LA (SEQ ID NO:13), wherein X₅ is Q, X₆ is S, X₇ is L, X₈ is Y, X₉ is selected from S or P, X₁₀ is N, X₁₁ is N, X₁₂ is Q, X₁₃ is K; and X₁₄ is Y;
(ii) a LCVR CDR2 sequence having the amino acid sequence WX₁₅SX₁₆RX₁₇X₁₈, wherein X₁₅ is A, X₁₆ is T, X₁₇ is G, and X₁₈ is selected from S or E, and
(iii) a LCVR CDR3 sequence having the amino acid sequence QQYYX₁₉X₂₀PFT (SEQ ID NO:30), wherein X₁₉ is S, and X₂₀ is selected from Y or F.

3. The isolated monoclonal antibody or an antigen-binding fragment thereof of claim 1 comprising:
(a) a HCVR, comprising a HCVR CDR1 sequence of SEQ ID NO:3, a HCVR CDR2 sequence of SEQ ID NO:10, and a HCVR CDR3 sequence of SEQ ID NO:12; and a LCVR, including a LCVR CDR1 sequence of SEQ ID NO:15, a LCVR CDR2 sequence of SEQ ID NO:23, and a LCVR CDR3 sequence of SEQ ID NO:32; or
(b) a HCVR, comprising a HCVR CDR1 sequence of SEQ ID NO:4, a HCVR CDR2 sequence of SEQ ID NO:11, and a HCVR CDR3 sequence of SEQ ID NO:12; and, a light chain variable region (LCVR), including a LCVR CDR1 sequence of SEQ ID NO:14, a LCVR CDR2 sequence of SEQ ID NO:24, and a LCVR CDR3 sequence of SEQ ID NO:31.

4. (canceled)

5. The isolated monoclonal antibody or antigen-binding fragment thereof of claim 1, wherein the antibody comprises:
the HCVR sequence of SEQ ID NO:39 and the LCVR sequence of SEQ ID NO:41; or
the HCVR sequence of SEQ ID NO:43 and the LCVR sequence of SEQ ID NO:45

6. The isolated monoclonal antibody or an antigen-binding fragment thereof of claim 1 comprising:
(a) a HCVR, comprising
(i) a HCVR CDR1 sequence having the amino acid sequence GYX₁FX₂X₃YTIH (SEQ ID NO:1), wherein X₁ is selected from T, P, or A; X₂ is selected from T, S, or G; and X₃ is selected from S or D;
(ii) a HCVR CDR2 sequence having the amino acid sequence WFYPGSGSTX₄YAQKFQG (SEQ ID NO:8), wherein X₄ is E; and
(iii) a HCVR CDR3 sequence having the amino acid sequence HGGYDGFDY (SEQ ID NO:12); and
(b) a LCVR comprising:
(i) a LCVR CDR1 sequence having the amino acid sequence KSSX₅X₆X₇LX₈X₉X₁₀X₁₁X₁₂X₁₃N X₁₄LA (SEQ ID NO:13), wherein X₅ is selected from Q or R; X₆ is selected from S or N; X₇ is selected from L, V, or I; X₈ is selected from Y or W; X₉ is S, X₁₀ is selected from S, P, or A; X₁₁ is selected from N or H; X₁₂ is N or Y; X₁₃ is K or R; and X₁₄ is Y or H;
(ii) a LCVR CDR2 sequence having the amino acid sequence WX₁₅SX₁₆RX₁₇X₁₈, wherein X₁₅ is selected from A or G; X₁₆ is selected from T, N, M, or A; X₁₇ is selected from G or E; and X₁₈ is selected from S, Y, P, or T; and
(iii) a LCVR CDR3 sequence having the amino acid sequence QQYYX₁₉X₂₀PFT (SEQ ID NO:30), wherein X₁₉ is F, and X₂₀ is Y.

7. The isolated monoclonal antibody or an antigen-binding fragment thereof of claim 1 comprising:
(a) a HCVR, comprising a HCVR CDR1 sequence of SEQ ID NO:5, a HCVR CDR2 sequence of SEQ ID NO:9, and a HCVR CDR3 sequence of SEQ ID NO:12; and, a light chain variable region (LCVR), comprising a LCVR CDR1 sequence of SEQ ID NO:16, a LCVR CDR2 sequence of SEQ ID NO:25, and a LCVR CDR3 sequence of SEQ ID NO:33; or
(b) a HCVR, comprising a HCVR CDR1 sequence of SEQ ID NO:6, a HCVR CDR2 sequence of SEQ ID NO:9, and a HCVR CDR3 sequence of SEQ ID NO:12; and a LCVR, comprising a LCVR CDR1 sequence of SEQ ID NO:17, a LCVR CDR2 sequence of SEQ ID NO:26, and a LCVR CDR3 sequence of SEQ ID NO:33, or
(c) a HCVR, comprising a HCVR CDR1 sequence of SEQ ID NO:7, a HCVR CDR2 sequence of SEQ ID NO:9, and a HCVR CDR3 sequence of SEQ ID NO:12; and a LCVR, comprising a LCVR CDR1 sequence of SEQ ID NO:18, a LCVR CDR2 sequence of SEQ ID NO:27, and a LCVR CDR3 sequence of SEQ ID NO:33, or
(d) a HCVR, comprising a HCVR CDR1 sequence of SEQ ID NO:7, a HCVR CDR2 sequence of SEQ ID NO:9, and a HCVR CDR3 sequence of SEQ ID NO:12; and a LCVR, comprising a LCVR CDR1 sequence of SEQ ID NO:19, a LCVR CDR2 sequence of SEQ ID NO:28, and a LCVR CDR3 sequence of SEQ ID NO:33, or
(e) a HCVR, comprising a HCVR CDR1 sequence of SEQ ID NO:7, a HCVR CDR2 sequence of SEQ ID NO:9, and a HCVR CDR3 sequence of SEQ ID NO:12; and a LCVR, comprising a LCVR CDR1 sequence of SEQ ID NO:20, a LCVR CDR2 sequence of SEQ ID NO:29, and a LCVR CDR3 sequence of SEQ ID NO:33.

8. (canceled)

9. The isolated monoclonal antibody or antigen-binding fragment thereof of claim 1, wherein the antibody comprises:
the HCVR sequence of SEQ ID NO:47 and the LCVR sequence of SEQ ID NO:49;
the HCVR sequence of SEQ ID NO:51 and the LCVR sequence of SEQ ID NO: 53;
the HCVR sequence of SEQ ID NO:55 and the LCVR sequence of SEQ ID NO:57;
the HCVR sequence of SEQ ID NO:59 and the LCVR sequence of SEQ ID NO:61; or
the HCVR sequence of SEQ ID NO:63 and the LCVR sequence of SEQ ID NO:65.

10. The isolated monoclonal antibody or an antigen-binding fragment thereof of claim 1 comprising:
(a) a HCVR, comprising
(i) a HCVR CDR1 sequence having the amino acid sequence GYX₁FX₂X₃YTIH (SEQ ID NO:1), wherein X₁ is selected from E or P; X₂ is selected from T or S; and X₃ selected from E or S;
(ii) a HCVR CDR2 sequence having the amino acid sequence WF YPGSGSTX₄YAQKFQG (SEQ ID NO:8), wherein X₄ is selected from W or E; and
(iii) a HCVR CDR3 sequence having the amino acid sequence HGGYDGFDY (SEQ ID NO:12), and
(b) a LCVR, comprising:
(i) a LCVR CDR1 sequence having the amino acid sequence KSSX₅X₆X₇LX₈X₉X₁₀X₁₁X₁₂X₁₃NX₁₄LA (SEQ ID NO:13), wherein X₅ is Q, X₆ is selected from S or N, X₇ is selected from L or V, X₈ is selected from Y or W, X₉ is selected from P or S, X₁₀ is selected from N or S, X₁₁ is N, X₁₂ is selected from Q or N, X₁₃ is K; and X₁₄ is Y;
(ii) a LCVR CDR2 sequence having the amino acid sequence WX₁₅SX₁₆RX₁₇X₁₈, wherein X₁₅ is selected from A or G; X₁₆ is selected from T or N; X₁₇ is selected from G or E, and X₁₈ is S; and
(iii) a LCVR CDR3 sequence having the amino acid sequence QQYYX₁₉X₂₀PFT (SEQ ID NO:30), wherein X₁₉ is selected from S or F; and X₂₀ is selected from Y or F.

11. The isolated monoclonal antibody or an antigen-binding fragment thereof of claim 1, comprising:
(a) a HCVR, comprising a HCVR CDR1 sequence of SEQ ID NO:3, a HCVR CDR2 sequence of SEQ ID NO:10, and a HCVR CDR3 sequence of SEQ ID NO:12; and a LCVR, including a LCVR CDR1 sequence of SEQ ID NO:15, a LCVR CDR2 sequence of SEQ ID NO:23, and a LCVR CDR3 sequence of SEQ ID NO:32; or
(b) a HCVR, comprising a HCVR CDR1 sequence of SEQ ID NO:5, a HCVR CDR2 sequence of SEQ ID NO:9, and a HCVR CDR3 sequence of SEQ ID NO:12; and a light chain variable region (LCVR), comprising a LCVR CDR1 sequence of SEQ ID NO:16, a LCVR CDR2 sequence of SEQ ID NO:25, and a LCVR CDR3 sequence of SEQ ID NO:33.

12. The isolated monoclonal antibody or antigen-binding fragment thereof according to claim 1, wherein the antibody is a humanized antibody.

13. The isolated monoclonal antibody or antigen-binding fragment thereof according to claim 1, wherein the antigen-binding fragment thereof is an Fab, Fab′, F(ab′)2, F_{d}, single chain Fv or scFv, disulfide linked Fᵥ, V-NAR domain, IgNar, intrabody, IgGACH₂, minibody, F(ab′)3, tetrabody, triabody, diabody, domain antibody (dAb), DVD-Ig, Fcab, mAb₂, (scFv)₂, tandem scFv, DART®, TandAb, nanobody or scFv-Fc.

14. The isolated monoclonal antibody or antigen-binding fragment thereof of claim 1, wherein said monoclonal antibody or antigen-binding fragment thereof binds human Gal9 with a K_{d} of less than about 5 nM, 2 nM, 1 nM, 0.5 nM, 0.4 nM, 0.3 nM, 0.2 nM, 0.1 nM, 0.05 nM or 0.01 nM.

15. The isolated monoclonal antibody or antigen-binding fragment thereof of claim 1, wherein the monoclonal antibody or antigen-binding fragment thereof:
(a) binds to Gal9 and inhibits Gal9 binding to a Gal9 receptor;
(b) binds to Gal9 and does not inhibit Gal9 binding to a Gal9 receptor;
(c) reverses Gal9-induced Th1 apoptosis of T cells: or
(d) suppresses Gal9-induced Treg expansion.

16-18. (canceled)

19. A method of treating cancer in a subject in need thereof comprising administering to the subject an effective amount of the isolated monoclonal antibody or antigen-binding fragment thereof of claim 1, thereby treating cancer in the subject.

20. The method of claim 19, wherein the cancer is
(a) a hematological cancer selected from the group consisting of acute lymphoblastic leukemia (ALL), acute myeloid leukemia (AML), chronic lymphocytic leukemia, chronic myelogenous leukemia, and hairy cell leukemia, AIDS-related lymphoma, cutaneous T-cell lymphoma, Hodgkin lymphoma, Hodgkin lymphoma, mycosis fungoides, non-Hodgkin lymphoma, primary central nervous system lymphoma, Sézary syndrome, cutaneous T-Cell lymphoma, Waldenström macroglobulinemia, diffuse large B-Cell Lymphoma (DLBCL) and multiple myeloma: or
(b) a solid tumor selected from the group consisting of breast cancer, head and neck cancer, lung cancer, melanoma, uveal melanoma, colorectal cancer, renal carcinoma, urothelial cancer ovarian cancer, endometrial cancer, liver cancer, pancreatic cancer, cholangiocarcinoma, gastric cancer, gastroesophageal cancer, esophageal cancer, cervical cancer, head and neck squamous cancer, and prostate cancer.

21-22. (canceled)

23. The method of claim 19, further comprising administering to the patient
(a) a chemotherapeutic agent;
(b) an anti-angiogenesis agent;
(c) a growth inhibitory agent;
(d) an immune-oncology agent, wherein the immune-oncology agent is an anti-glucocorticoid-induced tumor necrosis factor receptor family-related protein (GITR) antibody;
(e) a checkpoint inhibitor, wherein the checkpoint inhibitor is an anti-PD-1 antibody; and/or
(f) an anti-neoplastic composition.

24-25. (canceled)

26. A polynucleotide encoding the heavy chain or the light chain or the antigen-binding portion thereof of claim 1.

27. A vector comprising the polynucleotide of claim 26, wherein the vector is an expression vector selected from the group consisting of a mammalian expression vector, a yeast expression vector, an insect expression vector, and a bacterial expression vector.

28. A method of rescuing or promoting effector T cell proliferation, enhancing effector T cell activity, and/or of identifying and treating cancer in a subject comprising:
(i) determining a level of Gal9 in a sample from the subject, and
(ii) administering to a subject having a level Gal9 higher than a reference level of Gal9 an effective amount of the isolated monoclonal antibody or antigen-binding fragment thereof of claim 1,
thereby rescuing or promoting effector T cells proliferation, enhancing effector T cell activity, and/or of identifying and treating cancer in a subject.

29-31. (canceled)

32. The method of am claim 28, wherein the cancer is
(a) hematological cancer selected from the group consisting of acute lymphoblastic leukemia (ALL), acute myeloid leukemia (AML), chronic lymphocytic leukemia, chronic myelogenous leukemia, and hairy cell leukemia, AIDS-related lymphoma, cutaneous T-cell lymphoma, Hodgkin lymphoma, Hodgkin lymphoma, mycosis fungoides, non-Hodgkin lymphoma, primary central nervous system lymphoma, Sézary syndrome, cutaneous T-Cell lymphoma, Waldenström macroglobulinemia, diffuse large B-Cell Lymphoma (DLBCL) and multiple myeloma; or
(b) a solid tumor selected from the group consisting of breast cancer, head and neck cancer, lung cancer, melanoma, uveal melanoma, colorectal cancer, renal carcinoma, urothelial cancer ovarian cancer, endometrial cancer, liver cancer, pancreatic cancer, cholangiocarcinoma, gastric cancer, gastroesophageal cancer, esophageal cancer, cervical cancer, head and neck squamous cancer, and prostate cancer.

33-34. (canceled)

35. The method of claim 28, wherein the sample is a blood sample, a plasma sample, a serum sample, a tumor biopsy sample, or a bone-marrow (BM)-derived mononuclear cell (MNC) sample.

36. The method of claim 28, further comprising administering to the patient
(a) chemotherapeutic agent;
(b) an anti-angiogenesis agent;
(c) growth inhibitory agent;
(d) an immune-oncology agent, wherein the immune-oncology agent is an anti-glucocorticoid-induced tumor necrosis factor receptor family-related protein (GITR) antibody;
(e) a checkpoint inhibitor, wherein the checkpoint inhibitor is an anti-PD-1 antibody; and/or
(f) an anti-neoplastic composition.

37-38. (canceled)


SEQUENCE LISTING (77 sequences)

SEQ ID NO: 1 - protein, 10 aa, synthetic construct
  VARIANT 3: note = T, E, P, or A
  VARIANT 5: note = T, G, or S
  VARIANT 6: note = E, S, or D
GYXFXXYTIH

SEQ ID NO: 2 - protein, 10 aa, synthetic construct
GYTFTEYTIH

SEQ ID NO: 3 - protein, 10 aa, synthetic construct
GYEFTEYTIH

SEQ ID NO: 4 - protein, 10 aa, synthetic construct
GYTFGEYTIH

SEQ ID NO: 5 - protein, 10 aa, synthetic construct
GYPFSSYTIH

SEQ ID NO: 6 - protein, 10 aa, synthetic construct
GYAFTDYTIH

SEQ ID NO: 7 - protein, 10 aa, synthetic construct
GYTFGDYTIH

SEQ ID NO: 8 - protein, 17 aa, synthetic construct
  VARIANT 10: note = E, W, or V
WFYPGSGSTXYAQKFQG

SEQ ID NO: 9 - protein, 17 aa, synthetic construct
WFYPGSGSTEYAQKFQG

SEQ ID NO: 10 - protein, 17 aa, synthetic construct
WFYPGSGSTWYAQKFQG

SEQ ID NO: 11 - protein, 17 aa, synthetic construct
WFYPGSGSTVYAQKFQG

SEQ ID NO: 12 - protein, 9 aa, synthetic construct
HGGYDGFDY

SEQ ID NO: 13 - protein, 17 aa, synthetic construct
  VARIANT 4: note = Q or R
  VARIANT 5: note = S or N
  VARIANT 6: note = L, V, or I
  VARIANT 8: note = Y or W
  VARIANT 9: note = S or P
  VARIANT 10: note = N, S, P, or A
  VARIANT 11: note = N or H
  VARIANT 12: note = Q, N, or Y
  VARIANT 13: note = K or R
  VARIANT 15: note = Y or H
KSSXXXLXXXXXXNXLA

SEQ ID NO: 14 - protein, 17 aa, synthetic construct
KSSQSLLYSNNQKNYLA

SEQ ID NO: 15 - protein, 17 aa, synthetic construct
KSSQSLLYPNNQKNYLA

SEQ ID NO: 16 - protein, 17 aa, synthetic construct
KSSQNVLWSSNNKNYLA

SEQ ID NO: 17 - protein, 17 aa, synthetic construct
KSSRNILYSPHNKNYLA

SEQ ID NO: 18 - protein, 17 aa, synthetic construct
KSSQSVLYSANNRNYLA

SEQ ID NO: 19 - protein, 17 aa, synthetic construct
KSSQSLLYSSNYKNHLA

SEQ ID NO: 20 - protein, 17 aa, synthetic construct
KSSQNVLWSSNNRNYLA

SEQ ID NO: 21 - skipped

SEQ ID NO: 22 - protein, 7 aa, synthetic construct
WASTRGS

SEQ ID NO: 23 - protein, 7 aa, synthetic construct
WASTRGS

SEQ ID NO: 24 - protein, 7 aa, synthetic construct
WASTRGE

SEQ ID NO: 25 - protein, 7 aa, synthetic construct
WGSNRES

SEQ ID NO: 26 - protein, 7 aa, synthetic construct
WASMRGS

SEQ ID NO: 27 - protein, 7 aa, synthetic construct
WGSAREY

SEQ ID NO: 28 - protein, 7 aa, synthetic construct
WGSTREP

SEQ ID NO: 29 - protein, 7 aa, synthetic construct
WGSTRET

SEQ ID NO: 30 - protein, 9 aa, synthetic construct
  VARIANT 5: note = S or F
  VARIANT 6: note = Y or F
QQYYXXPFT

SEQ ID NO: 31 - protein, 9 aa, synthetic construct
QQYYSYPFT

SEQ ID NO: 32 - protein, 9 aa, synthetic construct
QQYYSFPFT

SEQ ID NO: 33 - protein, 9 aa, synthetic construct
QQYYFYPFT

SEQ ID NO: 34 - protein, 467 aa, synthetic construct
MGWSCIILFLVATATGAHSQVQLVQSGAEVKKPGASVKVSCKASGYTFTEYTIHWVRQAPGQGLEWMGWFYPGSGSTEYAQKFQGRVTMTADTSISTAYMELSRLRSDDTAVYFCERHGGYDGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 118 aa, synthetic construct
QVQLVQSGAEVKKPGASVKVSCKASGYTFTEYTIHWVRQAPGQGLEWMGWFYPGSGSTEYAQKFQGRVTMTADTSISTAYMELSRLRSDDTAVYFCERHGGYDGFDYWGQGTTVTVSS

SEQ ID NO: 36 - protein, 239 aa, synthetic construct
MGWSCIILFLVATATGAHSDIVMTQSPDSLAVSLGERATINCKSSQSLLYSNNQKNYLAWYQQKPGQPPKLLIYWASTRGSGVPDRFSGSGSGTDFTLTISSLQAEDVAVYYCQQYYSYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 113 aa, synthetic construct
DIVMTQSPDSLAVSLGERATINCKSSQSLLYSNNQKNYLAWYQQKPGQPPKLLIYWASTRGSGVPDRFSGSGSGTDFTLTISSLQAEDVAVYYCQQYYSYPFTFGGGTKVEIK

SEQ ID NO: 38 - protein, 467 aa, synthetic construct
MGWSCIILFLVATATGAHSQVQLVQSGAEVKKPGASVKVSCKASGYEFTEYTIHWVRQAPGQGLEWMGWFYPGSGSTWYAQKFQGRVTMTADTSISTAYMELSRLRSDDTAVYFCERHGGYDGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 118 aa, synthetic construct
QVQLVQSGAEVKKPGASVKVSCKASGYEFTEYTIHWVRQAPGQGLEWMGWFYPGSGSTWYAQKFQGRVTMTADTSISTAYMELSRLRSDDTAVYFCERHGGYDGFDYWGQGTTVTVSS

SEQ ID NO: 40 - protein, 239 aa, synthetic construct
MGWSCIILFLVATATGAHSDIVMTQSPDSLAVSLGERATINCKSSQSLLYPNNQKNYLAWYQQKPGQPPKLLIYWASTRGSGVPDRFSGSGSGTDFTLTISSLQAEDVAVYYCQQYYSFPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 113 aa, synthetic construct
DIVMTQSPDSLAVSLGERATINCKSSQSLLYPNNQKNYLAWYQQKPGQPPKLLIYWASTRGSGVPDRFSGSGSGTDFTLTISSLQAEDVAVYYCQQYYSFPFTFGGGTKVEIK

SEQ ID NO: 42 - protein, 467 aa, synthetic construct
MGWSCIILFLVATATGAHSQVQLVQSGAEVKKPGASVKVSCKASGYTFGEYTIHWVRQAPGQGLEWMGWFYPGSGSTVYAQKFQGRVTMTADTSISTAYMELSRLRSDDTAVYFCERHGGYDGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 118 aa, synthetic construct
QVQLVQSGAEVKKPGASVKVSCKASGYTFGEYTIHWVRQAPGQGLEWMGWFYPGSGSTVYAQKFQGRVTMTADTSISTAYMELSRLRSDDTAVYFCERHGGYDGFDYWGQGTTVTVSS

SEQ ID NO: 44 - protein, 239 aa, synthetic construct
MGWSCIILFLVATATGAHSDIVMTQSPDSLAVSLGERATINCKSSQSLLYSNNQKNYLAWYQQKPGQPPKLLIYWASTRGEGVPDRFSGSGSGTDFTLTISSLQAEDVAVYYCQQYYSYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 113 aa, synthetic construct
DIVMTQSPDSLAVSLGERATINCKSSQSLLYSNNQKNYLAWYQQKPGQPPKLLIYWASTRGEGVPDRFSGSGSGTDFTLTISSLQAEDVAVYYCQQYYSYPFTFGGGTKVEIK

SEQ ID NO: 46 - protein, 467 aa, synthetic construct
MGWSCIILFLVATATGAHSQVQLVQSGAEVKKPGASVKVSCKASGYPFSSYTIHWVRQAPGQGLEWMGWFYPGSGSTEYAQKFQGRVTMTADTSISTAYMELSRLRSDDTAVYFCEVHGGYDGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 118 aa, synthetic construct
QVQLVQSGAEVKKPGASVKVSCKASGYPFSSYTIHWVRQAPGQGLEWMGWFYPGSGSTEYAQKFQGRVTMTADTSISTAYMELSRLRSDDTAVYFCEVHGGYDGFDYWGQGTTVTVSS

SEQ ID NO: 48 - protein, 239 aa, synthetic construct
MGWSCIILFLVATATGAHSDIVMTQSPDSLAVSLGERATINCKSSQNVLWSSNNKNYLAWYQQKPGQPPKLLIYWGSNRESGVPDRFSGSGSGTDFTLTISSLQAEDVAVYYCQQYYFYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 113 aa, synthetic construct
DIVMTQSPDSLAVSLGERATINCKSSQNVLWSSNNKNYLAWYQQKPGQPPKLLIYWGSNRESGVPDRFSGSGSGTDFTLTISSLQAEDVAVYYCQQYYFYPFTFGGGTKVEIK

SEQ ID NO: 50 - protein, 467 aa, synthetic construct
MGWSCIILFLVATATGAHSQVQLVQSGAEVKKPGASVKVSCKASGYAFTDYTIHWVRQAPGQGLEWMGWFYPGSGSTEYAQKFQGRVTMTADTSISTAYMELSRLRSDDTAVYYCEVHGGYDGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 118 aa, synthetic construct
QVQLVQSGAEVKKPGASVKVSCKASGYAFTDYTIHWVRQAPGQGLEWMGWFYPGSGSTEYAQKFQGRVTMTADTSISTAYMELSRLRSDDTAVYYCEVHGGYDGFDYWGQGTTVTVSS

SEQ ID NO: 52 - protein, 239 aa, synthetic construct
MGWSCIILFLVATATGAHSDIVMTQSPDSLAVSLGERATINCKSSRNILYSPHNKNYLAWYQQKPGQPPKLLIYWASMRGSGVPDRFSGSGSGTDFTLTISSLQAEDVAVYYCQQYYFYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 113 aa, synthetic construct
DIVMTQSPDSLAVSLGERATINCKSSRNILYSPHNKNYLAWYQQKPGQPPKLLIYWASMRGSGVPDRFSGSGSGTDFTLTISSLQAEDVAVYYCQQYYFYPFTFGGGTKVEIK

SEQ ID NO: 54 - protein, 467 aa, synthetic construct
MGWSCIILFLVATATGAHSQVQLVQSGAEVKKPGASVKVSCKASGYTFGDYTIHWVRQAPGQGLEWMGWFYPGSGSTEYAQKFQGRVTMTADTSISTAYMELSRLRSDDTAVYYCEIHGGYDGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 118 aa, synthetic construct
QVQLVQSGAEVKKPGASVKVSCKASGYTFGDYTIHWVRQAPGQGLEWMGWFYPGSGSTEYAQKFQGRVTMTADTSISTAYMELSRLRSDDTAVYYCEIHGGYDGFDYWGQGTTVTVSS

SEQ ID NO: 56 - protein, 239 aa, synthetic construct
MGWSCIILFLVATATGAHSDIVMTQSPDSLAVSLGERATINCKSSQSVLYSANNRNYLAWYQQKPGQPPKLLIYWGSAREYGVPDRFSGSGSGTDFTLTISSLQAEDVAVYYCQQYYFYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 113 aa, synthetic construct
DIVMTQSPDSLAVSLGERATINCKSSQSVLYSANNRNYLAWYQQKPGQPPKLLIYWGSAREYGVPDRFSGSGSGTDFTLTISSLQAEDVAVYYCQQYYFYPFTFGGGTKVEIK

SEQ ID NO: 58 - protein, 467 aa, synthetic construct
MGWSCIILFLVATATGAHSQVQLVQSGAEVKKPGASVKVSCKASGYTFGDYTIHWVRQAPGQGLEWMGWFYPGSGSTEYAQKFQGRVTMTADTSISTAYMELSRLRSDDTAVYFCEIHGGYDGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 118 aa, synthetic construct
QVQLVQSGAEVKKPGASVKVSCKASGYTFGDYTIHWVRQAPGQGLEWMGWFYPGSGSTEYAQKFQGRVTMTADTSISTAYMELSRLRSDDTAVYFCEIHGGYDGFDYWGQGTTVTVSS

SEQ ID NO: 60 - protein, 239 aa, synthetic construct
MGWSCIILFLVATATGAHSDIVMTQSPDSLAVSLGERATINCKSSQSLLYSSNYKNHLAWYQQKPGQPPKLLIYWGSTREPGVPDRFSGSGSGTDFTLTISSLQAEDVAVYYCQQYYFYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 113 aa, synthetic construct
DIVMTQSPDSLAVSLGERATINCKSSQSLLYSSNYKNHLAWYQQKPGQPPKLLIYWGSTREPGVPDRFSGSGSGTDFTLTISSLQAEDVAVYYCQQYYFYPFTFGGGTKVEIK

SEQ ID NO: 62 - protein, 467 aa, synthetic construct; identical to SEQ ID NO: 54

SEQ ID NO: 63 - protein, 118 aa, synthetic construct; identical to SEQ ID NO: 55

SEQ ID NO: 64 - protein, 239 aa, synthetic construct
MGWSCIILFLVATATGAHSDIVMTQSPDSLAVSLGERATINCKSSQNVLWSSNNRNYLAWYQQKPGQPPKLLIYWGSTRETGVPDRFSGSGSGTDFTLTISSLQAEDVAVYYCQQYYFYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - protein, 113 aa, synthetic construct
DIVMTQSPDSLAVSLGERATINCKSSQNVLWSSNNRNYLAWYQQKPGQPPKLLIYWGSTRETGVPDRFSGSGSGTDFTLTISSLQAEDVAVYYCQQYYFYPFTFGGGTKVEIK

SEQ ID NO: 66 - protein, 25 aa, synthetic construct [sequence omitted]

SEQ ID NO: 67 - protein, 14 aa, synthetic construct
WVRQAPGQGLEWMG

SEQ ID NO: 68 - protein, 32 aa, synthetic construct
RVTMTADTSISTAYMELSRLRSDDTAVYFCER

SEQ ID NO: 69 - protein, 32 aa, synthetic construct
RVTMTADTSISTAYMELSRLRSDDTAVYFCEV

SEQ ID NO: 70 - protein, 32 aa, synthetic construct
RVTMTADTSISTAYMELSRLRSDDTAVYYCEI

SEQ ID NO: 71 - protein, 11 aa, synthetic construct
WGQGTTVTVSS

SEQ ID NO: 72 - protein, 23 aa, synthetic construct
DIVMTQSPDSLAVSLGERATINC

SEQ ID NO: 73 - protein, 15 aa, synthetic construct
WYQQKPGQPPKLLIY

SEQ ID NO: 74 - protein, 32 aa, synthetic construct
GVPDRFSGSGSGTDFTLTISSLQAEDVAVYYC

SEQ ID NO: 75 - protein, 10 aa, synthetic construct
FGGGTKVEIK

SEQ ID NO: 76 - protein, 32 aa, synthetic construct
RVTMTADTSISTAYMELSRLRSDDTAVYYCEV

SEQ ID NO: 77 - protein, 32 aa, synthetic construct
RVTMTADTSISTAYMELSRLRSDDTAVYFCEI